(19) United States
(12) Patent Application Publication
(10) Pub. No.: US 2023/0117370 A1
(43) Pub. Date: Apr. 20, 2023

(54) REGIOSELECTIVE SYNTHESIS OF SUBSTITUTED COMPOUNDS

(71) Applicant: Oregon State University, Corvallis, OR (US)

(72) Inventors: Christopher M. Beaudry, Corvallis, OR (US); Xiaojie Zhang, Corvallis, OR (US)

(73) Assignee: Oregon State University, Corvallis, OR (US)

(21) Appl. No.: 18/046,583

(22) Filed: Oct. 14, 2022

Related U.S. Application Data
(63) Continuation of application No. PCT/US2021/027705, filed on Apr. 16, 2021.
(60) Provisional application No. 63/011,770, filed on Apr. 17, 2020.

(51) Int. Cl. C07C 37/07; C07D 307/83

(52) U.S. Cl. CPC C07C 37/07; C07D 307/83

(57) ABSTRACT

Disclosed herein are embodiments of a method for making substituted compounds with specific and selectable regiochemistry. Also disclosed are compounds made by the method. The method may comprise contacting a compound having a formula I

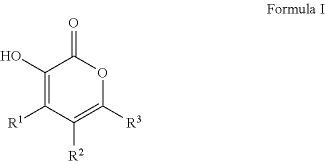

with a compound according to formula II

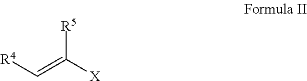

in the presence of a Lewis acid to form a phenol compound according to formula III and/or a benzofuranone compound according to formula IV

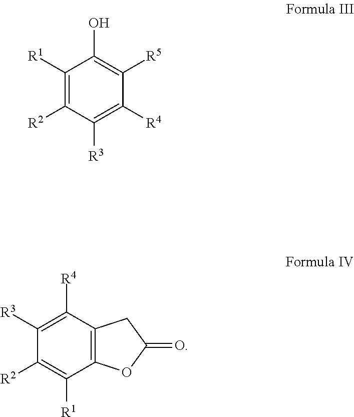

### CROSS REFERENCE TO RELATED APPLICATION

This application is a continuation of International Application No. PCT/US2021/027705, filed on Apr. 16, 2021, which claims the benefit of the earlier filing date of U.S. provisional patent application No. 63/011,770, filed Apr. 17, 2020, both of which are incorporated herein by reference in its entirety.

### ACKNOWLEDGMENT OF GOVERNMENT SUPPORT

This invention was made with government support under grant Nos. CHE-1465287 and CHE-1956401 awarded by National Science Foundation. The government has certain rights in the invention.

### FIELD

The present disclosure concerns a method for making substituted phenol compounds and compounds made by the method.

### BACKGROUND

Phenols are indispensable molecules. Substituted phenols represent essential pharmaceuticals, such as the analgesic morphine, the leukemia drug ecteinascidin, the hormonal birth control estrogen, and the general anesthetic propofol. Substituted phenols are also common agrochemicals (e.g., the citrus fungicide 2-phenylphenol and the food additive butylated hydroxytoluene), many are important substructures of biological polymers (e.g., lignin, tyrosine), and are used in human-made polymers such as the ubiquitous phenolic resin plastics. The properties of phenolic molecules are substantially influenced by the substitution on the phenolic ring. For these reasons, the synthesis of phenols with control of substituent regiochemistry is of paramount interest to organic, medicinal, and polymer chemists.

### SUMMARY

Disclosed herein is a method for making substituted compounds, such as phenol and benzofuranone compounds, with specific and selectable regiochemistry. In some embodiments, the method comprises contacting a first compound having a formula I

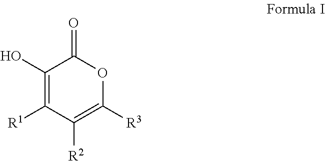

with a second compound according to formula II

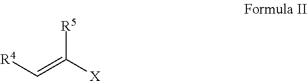

in the presence of a Lewis acid to form a third compound according to formula III

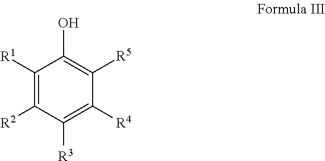

With respect to Formulas I, II and III, each of R¹, R², R³, R⁴ and R⁵ independently are H, aryl, aliphatic, heterocyclyl, alkoxy, heteroaliphatic, carboxylic ester, or —CH₂CO₂aliphatic, and X is nitro or SO₂Ph. Each of R¹, R², R³, R⁴ and R⁵ independently may be selected from H, alkyl, cycloalkyl, heteroaryl, heterocycloaliphatic, alkoxy, —CO₂R where R is aliphatic or hydroxyalkyl, or —CH₂CO₂alkyl, and in certain embodiments, each of R¹, R², R³, R⁴ and R⁵ independently is H or alkyl. In some embodiments, at least 3 of R¹, R², R³, R⁴ and R⁵ are not H and/or at least 3 of R¹, R², R³, R⁴ and R⁵ are different from each other.

In some embodiments, such as embodiments where R⁵ is —CH₂CO₂aliphatic, the method may comprise forming a fourth compound having a formula IV

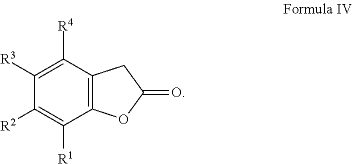

With respect to formula IV, R¹, R², R³ and R⁴ are as defined above for formula III. Forming the fourth compound may comprise allowing the reaction mixture from the formation of the third compound to continue to react to form the fourth compound. Additionally, or alternatively, forming the fourth compound may comprise exposing the third compound to a protic acid, such as, but not limited to, a haloacetic acid, such as trifluoroacetic acid; an aryl sulfonic acid, such as toluene sulfonic acid; a hydrohalide acid such as hydrochloric acid, hydrobromic acid or hydroiodic acid; or a combination thereof.

In some embodiments, the third compound is isolated and then exposed to the protic acid to form the fourth compound, but in other embodiments, the reaction is a one pot reaction and the third compound and the fourth compound are both formed in the presence of the Lewis acid and the protic acid.

In any embodiments, the Lewis acid may be FeCl₃, ZnCl₂, AlCl₃ or a combination thereof, and in some embodiments, the Lewis acid is, or comprises, AlCl₃. Additionally, or alternatively, contacting the first compound with a compound of Formula II occurs in the presence of both a Lewis acid and a radical initiator. The radical initiator may be butylated hydroxytoluene (BHT), hydroquinone, 2,6-di-tertbutyl phenol, or a combination thereof, and in certain embodiments, the radical initiator is butylated hydroxytoluene. And in particular embodiments, contacting the first compound with the compound according to formula II in the presence of AlCl₃ and butylated hydroxytoluene to form the phenol compound according to formula III.

Also disclosed are embodiments of a compound made by an embodiment of the disclosed method. In some embodiments, the compound has a structure according to Formulas III or IV, as described above. In certain embodiments, at least 3 of R¹, R², R³, R⁴ and R⁵ if present, are not H and at least two of the non-H substituents are different from each other. And/or in particular embodiments of a compound according to Formula III, the compound is not 2,6-dimethyl-3-phenylphenol.

In some embodiments, at least 4 of R¹, R², R³, R⁴ and R⁵, if present, are not H and at least two of the non-H substituents are different from each other, and in other embodiments, each of R¹, R², R³, R⁴ and RV, if present, are not H and at least two of the substituents are different from each other. In any embodiments, at least three of the non-H substituents may be different from each other, such as at least four of the non-H substituents being different from each other, or all substituents being different from each other.

In some embodiments, of a compound according to Formula IV, each of R¹, R², R³, and R⁴ independently are H, aryl, aliphatic, heterocyclyl, alkoxy, heteroaliphatic, CO₂H or carboxylic ester, with the proviso that at least two of R¹, R², R³, and R⁴ are not H, and one of conditions a), b) and c) applies:

a) If exactly two of R¹, R², R³, and R⁴ are not H then they are different from each other;

b) If exactly three of R¹, R², R³, and R⁴ are not H then at least two of the non-H substituents are different from each other; or

c) If all four of R¹, R², R³, and R⁴ are not H then at least two of R¹, R², R³, and R⁴ are different from each other.

Additionally, the compound is not a compound disclosed in Table 1.

The foregoing and other objects, features, and advantages of the invention will become more apparent from the following detailed description, which proceeds with reference to the accompanying figures.

### BRIEF DESCRIPTION OF THE DRAWINGS

FIG. 1 is a table illustrating various reaction conditions used in the disclosed phenol synthesis.

FIG. 2A is a table of exemplary substituted phenol compounds made by the disclosed method.

FIG. 2B is a second table of exemplary substituted phenol compounds made by the disclosed method.

FIG. 3 is a table illustrating a subset of the compounds from FIGS. 2A and 2B that to the inventors' knowledge, have not been previously synthesized with regioselectivity.

FIG. 4 is a table illustrating the conversion of certain substituted phenols into substituted benzene compounds.

FIG. 5 is a table illustrating various reaction conditions used in the disclosed benzofuranone synthesis.

FIG. 6 is a table of exemplary substituted benzofuranone compounds made by the disclosed method.

FIG. 7 is a table illustrating the conversion of certain substituted benzofuranone into substituted benzofuran compounds.

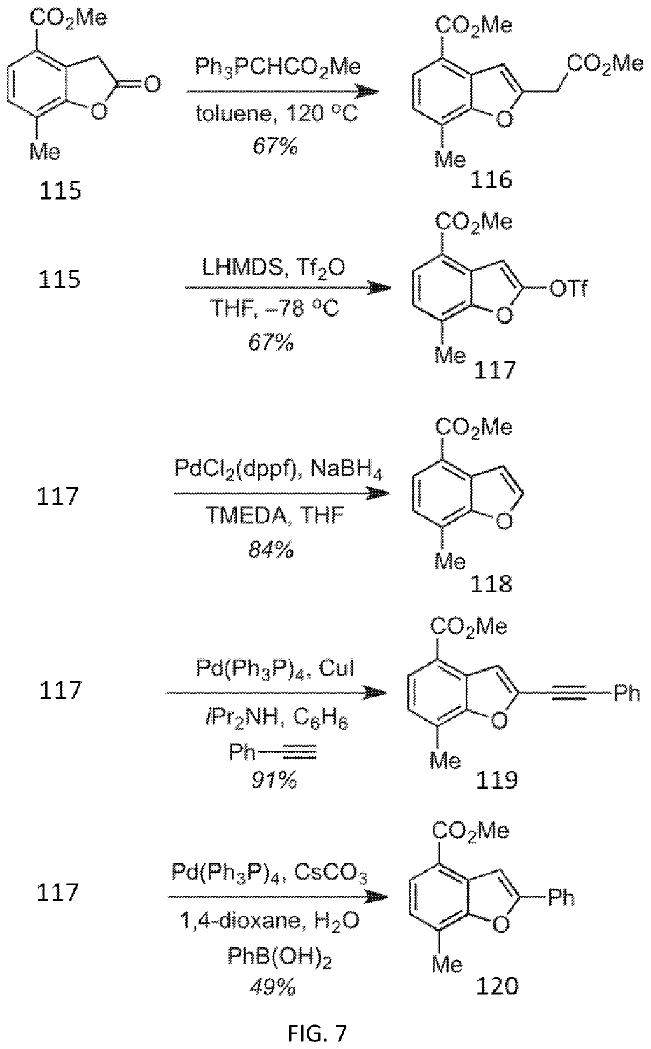

### DETAILED DESCRIPTION

### I. Definitions

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. All references, including patents and patent applications cited herein, are incorporated by reference in their entirety, unless otherwise specified.

Unless otherwise indicated, all numbers expressing quantities of components, molecular weights, percentages, temperatures, times, and so forth, as used in the specification or claims, are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is expressly recited.

Unless explained otherwise, all technical and scientific terms used herein have the same meaning as commonly understood by one of ordinary skill in the art to which this disclosure pertains. Although methods and materials similar or equivalent to those described herein can be used in the practice or testing of the present disclosure, suitable methods and materials are described below. The materials, methods, and examples are illustrative only and not intended to be limiting.

When chemical structures are depicted or described, unless explicitly stated otherwise, all carbons are assumed to include implicit hydrogens such that each carbon conforms to a valence of four. For example, in the structure on the left-hand side of the schematic below there are nine hydrogen atoms implied. The nine hydrogen atoms are depicted in the right-hand structure.

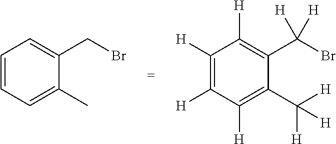

Sometimes a particular atom in a structure is described in textual formula as having a hydrogen or hydrogen atoms, for example —CH₂CH₂—. It will be understood by a person of ordinary skill in the art that the aforementioned descriptive techniques are common in the chemical arts to provide brevity and simplicity to description of organic structures.

“Substituted,” when used to modify a specified group or moiety, means that at least one, and perhaps two or more, hydrogen atoms of the specified group or moiety is independently replaced with the same or different substituent groups as defined below. In a particular embodiment, a group, moiety or substituent may be substituted or unsubstituted, unless expressly defined as either “unsubstituted” or “substituted.” Accordingly, any of the groups specified herein may be unsubstituted or substituted. In particular embodiments, the substituent may or may not be expressly defined as substituted, but is still contemplated to be optionally substituted. For example, an “alkyl” moiety may be unsubstituted or substituted, but an “unsubstituted alkyl” is not substituted.

As used herein, the term “substituted” refers to all subsequent modifiers in a term, for example in the term “substituted arylC₁₋₈alkyl,” substitution may occur on the “C₁₋₈alkyl” portion, the “aryl” portion or both portions of the arylC₁₋₈alkyl group.

“Substituents” or “substituent groups” for substituting for one or more hydrogen atoms the specified group or moiety are, unless otherwise specified, aliphatic, such as alkyl, alkenyl, or alkynyl, preferably C₁₋₄alkyl or C₁₋₄alkyl; hydroxyl; hydroxylalkyl; protected hydroxyl; protected hydroxyalkyl; alkoxy; aryl; heteroaryl; cycloaliphatic; CO₂H; carboxylic ester, such as CO₂R where R is alkyl; or a combination thereof.

In one embodiment, a group that is substituted has at least one substituent up to the number of substituents possible for a particular moiety, such as 1 substituent, 2 substituents, 3 substituents, or 4 substituents.

Additionally, in embodiments where a group or moiety is substituted with a substituted substituent, the nesting of such substituted substituents is limited to three, thereby preventing the formation of polymers. Thus, in a group or moiety comprising a first group that is a substituent on a second group that is itself a substituent on a third group, which is attached to the parent structure, the first (outermost) group can only be substituted with unsubstituted substituents. For example, in a group comprising -(aryl-1)-(aryl-2)-(aryl-3), aryl-3 can only be substituted with substituents that are not themselves substituted.

Any group or moiety defined herein can be connected to any other portion of a disclosed structure, such as a parent or core structure, as would be understood by a person of ordinary skill in the art, such as by considering valence rules, comparison to exemplary species, and/or considering functionality, unless the connectivity of the group or moiety to the other portion of the structure is expressly stated, or is implied by context.

“Aliphatic” refers to a substantially hydrocarbon-based group or moiety. An aliphatic group or moiety can be acyclic, including alkyl, alkenyl, or alkynyl groups, cyclic versions thereof, such as cycloaliphatic groups or moieties including cycloalkyl, cycloalkenyl or cycloalkynyl, and further including straight- and branched-chain arrangements, and all stereo and position isomers as well. Unless expressly stated otherwise, an aliphatic group contains from one to twenty-five carbon atoms (C₁₋₂₅); for example, from one to fifteen (C₁₋₁₅), from one to ten (C₁₋₁₀), from one to six (C₁₋₆), or from one to four carbon atoms (C₁₋₄) for a saturated acyclic aliphatic group or moiety, from two to twenty-five carbon atoms (C₂₋₂₅); for example, from two to fifteen (C₂₋₁₅), from two to ten (C₂₋₁₀), from two to six (C₂₋₆), or from two to four carbon atoms (C₂₋₄) for an unsaturated acyclic aliphatic group or moiety, or from three to fifteen (C₃₋₁₅) from three to ten (C₃₋₁₀), from three to six (C₃₋₆), or from three to four (C₃₋₄) carbon atoms for a cycloaliphatic group or moiety. An aliphatic group may be substituted or unsubstituted, unless expressly referred to as an “unsubstituted aliphatic” or a “substituted aliphatic.” An aliphatic group can be substituted with one or more substituents (up to two substituents for each methylene carbon in an aliphatic chain, or up to one substituent for each carbon of a —C═C— double bond in an aliphatic chain, or up to one substituent for a carbon of a terminal methine group).

“Lower aliphatic” refers to an aliphatic group containing from one to ten carbon atoms (C₁₋₁₀), such as from one to six (C₁₋₆), or from one to four (C₁₋₄) carbon atoms for a saturated acyclic lower aliphatic group or moiety; from two to ten (C₂₋₁₀), from two to six (C₂₋₆), or from two to four carbon atoms (C₂₋₄) for an unsaturated acyclic lower aliphatic group or moiety; or from three to ten (C₃₋₁₀), such as from three to six (C₃₋₆) carbon atoms for a lower cycloaliphatic group.

“Alkyl” refers to a saturated aliphatic hydrocarbyl group having from 1 to 25 (C₁₋₂₅) or more carbon atoms, more typically 1 to 10 (C₁₋₁₀) carbon atoms such as 1 to 6 (C₁₋₆) carbon atoms, 1 to 4 (C₁₋₄) carbon atoms, or 1 to 2 (C₁₋₂) carbon atoms. An alkyl moiety may be substituted or unsubstituted. This term includes, by way of example, linear and branched hydrocarbyl groups such as methyl (CH₃), ethyl (—CH₂CH₃), n-propyl (—CH₂CH₂CH₃), isopropyl (—CH(CH₃)₂), n-butyl (—CH₂CH₂CH₂CH₃), isobutyl (—CH₂CH₂(CH₃)₂), sec-butyl (—CH(CH₃)(CH₂CH₃), t-butyl (—C(CH₃)₃), n-pentyl (—CH₂CH₂CH₂CH₂CH₃), and neopentyl (—CH₂C(CH₃)₃).

“Aryl” refers to an aromatic carbocyclic group of, unless specified otherwise, from 6 to 15 carbon atoms having a single ring (e.g., phenyl) or multiple condensed rings in which at least one ring is aromatic (e.g., 1,2,3,4-tetrahydroquinoline, benzodioxole, and the like) providing that the point of attachment is through an aromatic portion of the ring system. If any aromatic ring portion contains a heteroatom, the group is heteroaryl and not aryl. Aryl groups may be, for example, monocyclic, bicyclic, tricyclic or tetracyclic. Unless otherwise stated, an aryl group may be substituted or unsubstituted.

“Carboxylic ester” refers to the group —COOR, where R is aliphatic, aryl, heterocyclyl, typically alkyl.

“Carboxyl” or “carboxylic acid” refers to the group —COOH, i.e., —COOR where R is H.

“Heteroaliphatic” refers to an aliphatic moiety or group having at least one heteroatom and at least one carbon atom, i.e., one or more carbon atoms from an aliphatic compound or group comprising at least two carbon atoms, has been replaced with an atom having at least one lone pair of electrons, typically nitrogen, oxygen, phosphorus, silicon, or sulfur. In some embodiment, the point of attachment to the parent structure is through a carbon atom in the heteroaliphatic moiety, i.e., the point of attachment is not through a heteroatom. In some embodiments, a heteroaliphatic moiety or group has terminal carbon atoms, i.e., the heteroaliphatic moiety or group comprises at least 2 carbon atoms and at least one heteroatom, and both the atom at the point of attachment to the parent structure and the terminal atom(s) are carbon atoms. Heteroaliphatic compounds or groups may be substituted or unsubstituted, branched or unbranched, chiral or achiral, and/or acyclic or cyclic, such as a heterocycloaliphatic group.

“Heteroaryl” refers to an aromatic group or moiety of, unless specified otherwise, from 5 to 15 ring atoms comprising at least one carbon atom and at least one heteroatom, such as N, S, or O. A heteroaryl group or moiety may comprise a single ring (e.g., pyridinyl, pyrimidinyl or imidazolyl) or multiple condensed rings (e.g., indolyl or benzimidazolyl). Heteroaryl groups or moiety may be, for example, monocyclic, bicyclic, tricyclic or tetracyclic. Unless otherwise stated, a heteroaryl group or moiety may be substituted or unsubstituted.

“Heterocyclyl,” “heterocyclo” and “heterocycle” refer to both aromatic and non-aromatic ring systems, and more specifically refer to a stable three- to fifteen-membered ring moiety comprising at least one carbon atom, and typically plural carbon atoms, and at least one, such as from one to five, heteroatoms. The heteroatom(s) may be nitrogen, oxygen, sulfur, phosphorus, or silicon atom(s), preferably nitrogen, oxygen, or sulfur atom(s). The heterocyclyl moiety may be a monocyclic moiety, or may comprise multiple rings, such as in a bicyclic or tricyclic ring system, provided that at least one of the rings contains a heteroatom. Such a multiple ring moiety can include fused or bridged ring systems as well as spirocyclic systems, and may include all aromatic, all non-aromatic, or both aromatic and non-aromatic rings. Also, any nitrogen, carbon, or sulfur atoms in the heterocyclyl moiety can be optionally oxidized to various oxidation states. For convenience, nitrogens, particularly, but not exclusively, those defined as annular aromatic nitrogens, are meant to include their corresponding N-oxide form, although not explicitly defined as such in a particular example. Thus, for a compound having, for example, a pyridinyl ring, the corresponding pyridinyl-N-oxide is included as another compound of the invention, unless expressly excluded or excluded by context. In addition, annular nitrogen atoms can be optionally quaternized. Unless otherwise stated, a heterocyclyl moiety may be substituted or unsubstituted. Heterocycle includes heteroaryl moieties, and non-aromatic heterocyclyl moieties, also called heterocycloaliphatic moieties, which may be partially or fully saturated rings. Examples of heterocyclyl groups include, but are not limited to, azetidinyl, oxetanyl, acridinyl, benzodioxolyl, benzodioxanyl, benzofuranyl, carbazoyl, cinnolinyl, dioxolanyl, indolizinyl, naphthyridinyl, perhydroazepinyl, phenazinyl, phenothiazinyl, phenoxazinyl, phthalazinyl, pteridinyl, purinyl, quinazolinyl, quinoxalinyl, quinolinyl, isoquinolinyl, tetrazoyl, tetrahydroisoquinolyl, piperidinyl, piperazinyl, 2-oxopiperazinyl, 2-oxopiperidinyl, 2-oxopyrrolidinyl, 2-oxoazepinyl, azepinyl, pyrrolyl, 4-piperidonyl, pyrrolidinyl, pyrazolyl, pyrazolidinyl, imidazolyl, imidazolinyl, imidazolidinyl, dihydropyridinyl, tetrahydropyridinyl, pyridinyl, pyrazinyl, pyrimidinyl, pyridazinyl, oxazolyl, oxazolinyl, oxazolidinyl, triazolyl, isoxazolyl, isoxazolidinyl, morpholinyl, thiazolyl, thiazolinyl, thiazolidinyl, isothiazolyl, quinuclidinyl, isothiazolidinyl, indolyl, isoindolyl, indolinyl, isoindolinyl, octahydroindolyl, octahydroisoindolyl, quinolyl, isoquinolyl, decahydroisoquinolyl, benzimidazolyl, thiadiazolyl, benzopyranyl, benzothiazolyl, benzoxazolyl, furyl, diazabicycloheptane, diazapane, diazepine, tetrahydrofuryl, tetrahydropyranyl, thienyl, benzothieliyl, thiamorpholinyl, thiamorpholinyl sulfoxide, thiamorpholinyl sulfone, dioxaphospholanyl, and oxadiazolyl.

“Hydroxyalkyl” refers to an alkyl moiety substituted with one or more hydroxyl groups. A protected hydroxyalkyl is a hydroxyalkyl where at least one hydroxyl moiety is protected by a protecting group (PG), such as a protecting group disclosed herein. Exemplary hydroxyalkyl moieties include —CH₂OH, —CH₂CH₂OH, —CH(OH)CH₃,

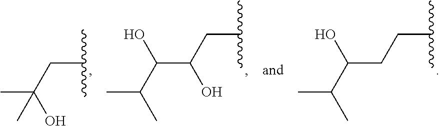

“Lewis acid” refers to an electron pair acceptor. Typically, Lewis acids have an unoccupied low-energy atomic or molecular orbital. A Lewis acid may be an ion or an uncharged species. Exemplary Lewis acids include, but are not limited to, H⁺, K⁺, Mg²⁺, Fe³⁺, BF₃, CO₂, SO₃, AlCl₃, Br₂, LiClO₄, SiO₂, FeCl₃, ZnCl₂, Zn(OTf)₂, and combinations thereof.

“Protecting group” refers to any protecting group known to a person of ordinary skill in the art as suitable to protect a particular moiety, such as a hydroxyl or amino moiety. Suitable protecting groups, and methods for attaching and removing such groups, are known to persons of ordinary skill in the art, and additional information concerning such groups can be found in Greene, Protective Groups in Organic Synthesis; 4th Ed.; John Wiley & Sons, New York, 2014, which is incorporated herein by reference. Exemplary protecting groups include, but are not limited to, acetate, chloroacetate, dichloroacetate, trichloroacetate, trifluoroacetate, pivaloate, benzoate, p-methoxybenzoate, p-bromobenzoate, methyl carbonate, 9-(fluorenylmethyl) carbonate (Fmoc), allyl carbonate, benzyl carbonate (CBZ), t-butyl carbonate (Boc), dimethylthiocarbonate (DMTC), methoxymethyl (MOM), tert-butyl, iso-propyl, and silyl protecting groups, such as tert-butyldimethylsilyl (TBS or TBDMS), trimethylsilyl (TMS), triethylsilyl (TES), tert-butyldiphenylsilyl (TBDPS), or triisopropylsilyl (TIPS).

“Solvate” refers to a complex formed by combination of solvent molecules with molecules or ions of the solute. The solvent can be an organic compound, an inorganic compound, or a mixture of both. Some examples of solvents include, but are not limited to, methanol, N,N-dimethylformamide, tetrahydrofuran, dimethylsulfoxide, and water. The compounds described herein can exist in un-solvated as well as solvated forms when combined with solvents, pharmaceutically acceptable or not, such as water, ethanol, and the like. When a compound forms a solvate with water it may be referred to as a hydrate. Solvated forms of the presently disclosed compounds are within the scope of the embodiments disclosed herein.

E/Z isomers: Isomers that differ in the stereochemistry of a double bond. An E isomer (from entgegen, the German word for “opposite”) has a trans-configuration at the double bond, in which the two groups of highest priority are on opposite sides of the double bond. A Z isomer (from zusammen, the German word for “together”) has a cis-configuration at the double bond, in which the two groups of highest priority are on the same side of the double bond. The E and Z isomers of 2-butene are shown below:

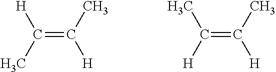

Unless otherwise specified, a non-ring double bond may be the E isomer, the Z isomer, or a mixture thereof. A double bond may be shown with one substituent attached by a wavy bond. This indicates that the stereochemistry of the substituent is not specified and can be E, Z or a mixture thereof:

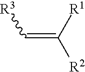

However, a person of ordinary skill in the art understands that even if the stereochemistry of a double bond in a compound is shown as either the E or Z isomer, the isomer is provided as an example and the other isomer also is contemplated, unless the context dictates otherwise.

In any embodiments, any or all hydrogens present in the compound, or in a particular group or moiety within the compound, may be replaced by a deuterium or a tritium. Thus, a recitation of alkyl includes deuterated alkyl, where from one to the maximum number of hydrogens present may be replaced by deuterium. For example, ethyl may be C₂H₅ or C₂H₅ where from 1 to 5 hydrogens are replaced by deuterium, such as in C₂DₓH₅₋ₓ. In certain embodiments, a deuterated compound comprises an amount of deuterium at a specified position that is greater than an amount of deuterium that might be present at that position due to the natural abundance of deuterium had the compound been a natural product rather than synthetically produced.

### II. Overview

The chemical synthesis of phenols with complex substitution patterns is an enduring challenge, and multiple strategies have emerged for this task (Schemes 1-3). However, current methods have substantial limitations. For example, classic alkylations (e.g., Friedel-Crafts alkylations) of unprotected phenols give phenyl ethers, rather than substituted phenols (Scheme 1). Substitution of phenyl ethers gives multiple products as a result of limited regioselectivity (ortho- vs para-substitution) accompanied by over-alkylation products.

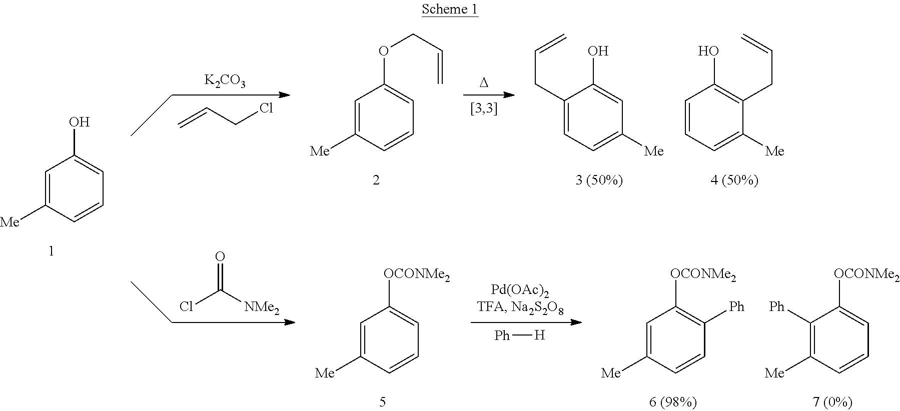

When regioselective phenol synthesis is required, a common strategy features rearrangement of O-bound groups to the adjacent ortho-position. In a classic example, allyl phenyl ethers (2) undergo Claisen rearrangements to reliably give ortho-substituted products 3 and 4 (Scheme 1, top). When both ortho-positions are unsubstituted, selectivity (3 vs 4) tend to be low. Other classic reactions (e.g., the Fries rearrangement) also follow this trend. There has been recent activity directed at C—H bond functionalization of phenol derivatives. As one example, phenol derivative 5 contains a suitable directing group, which facilitates Pd-catalyzed bond formation at the ortho-position (Scheme 1, bottom). In substrates such as 5 with two available ortho-positions, the contra-steric product 6 is obtained, and the product of vicinal substitution (7) is not observed. To summarize decades of work on substitution of phenol derivatives: such reactions produce limited regiochemical outcomes that disfavor vicinal substitution, making highly substituted phenols, such as those with 3, 4 or 5 substituents, inaccessible by these routes.

Limitations associated with substitution-based strategies for phenol syntheses have spawned a variety of alternative syntheses. One strategy is to oxidize a corresponding six-membered carbocycle to a phenol. For example, it has been reported that cyclohexanone 8 undergoes oxidative aromatization to give substituted phenol 9, and that substituted benzenes, such as mesitylene (10), can be oxidized to phenols (11) by direct oxidation (Scheme 2).

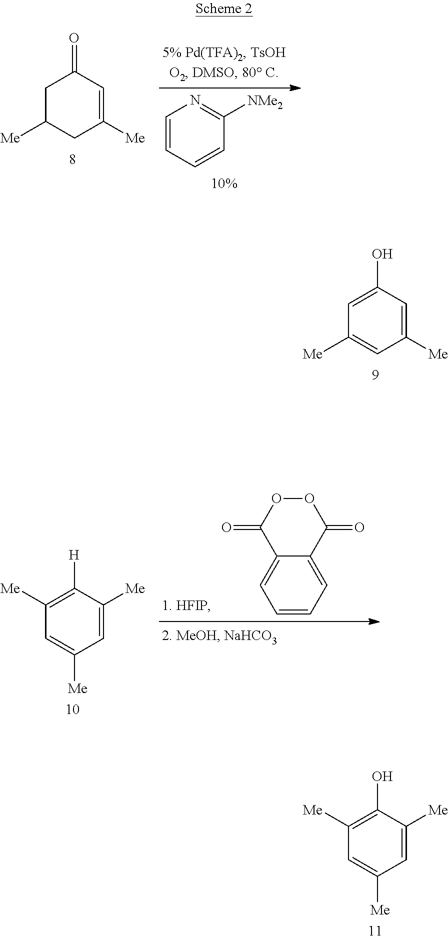

When multiple benzene C—H bonds are present, oxidation regiochemistry is based on C—H bond strength, and regioselectivities can be modest, and these oxidative methods have not resulted in a phenol synthesis that produces highly substituted (for example, penta-substituted) phenols with control of substitution patterns.

Another strategy is to use a cycloaddition cascade to prepare the phenol. The Diels-Alder reaction is commonly featured in such cascades. A substituted diene, such as pyrone 12 (Scheme 3), or other weakly aromatic heterocycle, reacts with a dienophile, most often an alkyne such as 13, to give the phenyl ether 14. This reaction proceeds by a Diels-Alder-retro-Diels-Alder sequence. Non-symmetric internal alkynes are not particularly polarized, and low regioselectivities are observed in the initial Diels-Alder event. Regioselectivities aside, these reactions are predominantly used to prepare simple mono- and di-substituted phenols.

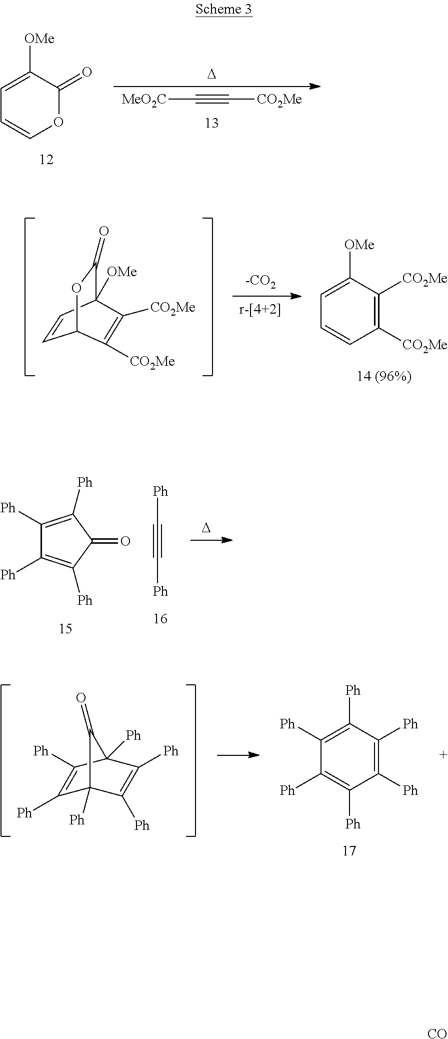

Diels-Alder reactions of highly reactive dienes are known to undergo cycloaddition with alkynes to give benzenes with high levels of substitution, including hexa-substituted benzenes. For example, heating 2,3,4,5-tetraphenyl cyclopentadiene (15) with diphenylacetylene (16) gives hexaphenyl benzene (17, Scheme 3). The limitation with these reactions is the lack of regioselectivity; both the diene and dienophile lack strong electronic polarization, and the reaction gives either symmetric products or regiochemical mixtures that require time consuming and often expensive separations, and often result in low yields of a desired isomer.

### III. Method for Making the Compounds

Disclosed herein are embodiments of a method for making phenol compounds and/or benzofuranone compounds. In some embodiments, the method can be used to make substituted phenol compounds, such as phenol compounds having 1, 2, 3, 4, or 5 substituents. And in certain embodiments, the phenol compound can undergo intramolecular cyclization to form a benzofuranone compound. The substituents on the phenol and/or benzofuranone compound may be all the same, all different, or a mixture of substituents. In certain embodiments, substitute phenols can be made in a regioselective manner, such that a person of ordinary skill in the art can select where the particular substituents will be in the final phenol or benzofuranone compound. In some embodiments, the method for making the phenol compounds follows the scheme shown in Scheme 4.

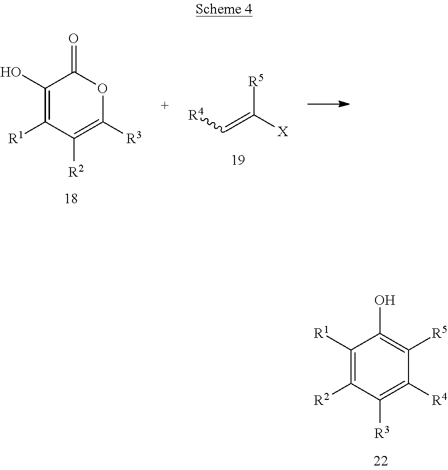

The reaction may proceed via an elimination pathway shown in Scheme 5.

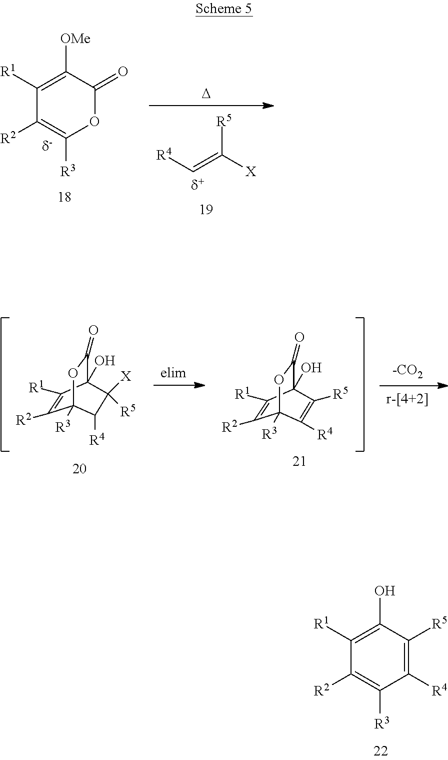

With respect to Schemes 4 and 5, each of R¹, R², R³, R⁴ and R⁵ independently are H; aryl; aliphatic, such as alkyl, alkenyl, alkynyl, cycloalkyl, cycloalkenyl, or cycloalkynyl; heterocyclyl, such as heteroaryl or heterocycloaliphatic; alkoxy; heteroaliphatic; —CH₂CO₂aliphatic; or carboxylic ester, such as —CO₂R where R is aliphatic or hydroxyalkyl, such as alkyl or hydroxyalkyl. In some embodiments, each of R¹, R², R³, R⁴ and R⁵ independently is H, alkyl, cycloalkyl, heteroaryl, heterocycloaliphatic, alkoxy or —CO₂R, and may be H, alkyl, aryl, or —CO₂R. And in certain embodiments, each of R¹, R², R³, R⁴ and R⁵ independently is H or alkyl.

However, in other embodiments, R⁵ is —CH₂CO₂alkyl, such as —CH₂CO₂C₁₋₆alkyl, and may be —CH₂CO₂methyl or —CH₂CO₂ethyl.

X is a leaving group and may be an electron withdrawing group, for example, nitro or a sulfonyl group such as SO₂Ph. In certain embodiments, X is nitro. In some embodiments, each of R¹, R², R³, R⁴ and R⁵ independently is H or alkyl. In any embodiments, at least one of R¹, R², R³, R⁴ and R⁵ is not H, such as at least 2 of R¹, R², R³, R⁴ and R⁵, at least 3 of R¹, R², R³, R⁴ and R⁵, at least 4 of R¹, R², R³, R⁴ and R⁵, or all 5 of R¹, R², R³, R⁴ and R⁵ are not H. In particular embodiments, at least 3 of R¹, R², R³, R⁴ and R⁵ are not H.

In any embodiments, at least 2 of R¹, R², R³, R⁴ and R⁵ are different from each other, such as at least 3 of R¹, R², R³, R⁴ and R⁵, at least 4 of R¹, R², R³, R⁴ and R⁵, or all 5 of R¹, R², R³, R⁴ and R⁵ are different from each other. In certain embodiments, at least 2 of R¹, R², R³, R⁴ and R⁵ are different from each other, and may be at least 3 of R¹, R², R³, R⁴ and R⁵ are different from each other.

In particular embodiments, at least 3 of R¹, R², R³, R⁴ and R⁵ are not H and at least two of the non-H substituents are different from each other; at least 4 of R¹, R², R³, R⁴ and R⁵ are not H and at least two of the non-H substituents are different from each other; or each of R¹, R², R³, R⁴ and R⁵ are not H and at least two of the substituents are different from each other. And in any embodiments, at least three of the non-H substituents are different from each other, or at least four of the non-H substituents are different from each other, or all five substituents are different from each other.

In embodiments where R⁵ is —CH₂CO₂aliphatic, the reaction may proceed according to Scheme 6

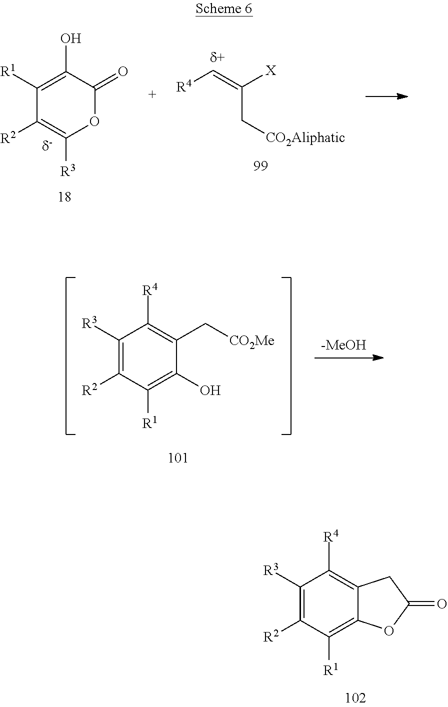

With respect to Scheme 6, X is as defined above with respect to Scheme 5, and R¹, R², R³, and R⁴ are each independently H; aryl; aliphatic, such as alkyl, alkenyl, alkynyl, cycloalkyl, cycloalkenyl, or cycloalkynyl; heterocyclyl, such as heteroaryl or heterocycloaliphatic; alkoxy; heteroaliphatic; or carboxylic ester, such as —CO₂R where R is aliphatic or hydroxyalkyl. In some embodiments, each of R¹, R², R³, and R⁴ independently is H, alkyl, cycloalkyl, heteroaryl, heterocycloaliphatic, alkoxy or CO₂R, such as H alkyl, aryl, or CO₂R. And in certain embodiments, each of R¹, R², R³, and R⁴ independently is H or alkyl. And in certain embodiments, the aliphatic moiety in compound 99 is alkyl, such as C₁₋₆alkyl, and in particular embodiments, is methyl or ethyl. And in certain embodiments of compound 99, X is nitro.

In some embodiments of Scheme 6, each of R¹, R², R³, and R⁴ independently is H or alkyl. In any embodiments, at least one of R¹, R², R³, and R⁴ is not H, such as at least 2 of R¹, R², R³, and R⁴, at least 3 of R¹, R², R³, and R⁴, or all 4 of R¹, R², R³, and R⁴ are not H. In particular embodiments, at least 3 of R¹, R², R³, and R⁴ are not H.

In any embodiments, at least 2 of R¹, R², R³, and R⁴ are different from each other, such as at least 3 of R¹, R², R³, and R⁴, or all 4 of R¹, R², R³, and R⁴ are different from each other. In certain embodiments, at least 2 of R¹, R², R³, and R⁴ are different from each other, and may be at least 3 of R¹, R², R³, and R⁴ are different from each other.

In particular embodiments, at least 3 of R¹, R², R³, and R⁴ are not H and at least two of the non-H substituents are different from each other; or each of R¹, R², R³, R⁴ and R⁵ are not H and at least two of the substituents are different from each other. And in any embodiments, at least three of the non-H substituents are different from each other, or all four of the non-H substituents are different from each other.

In any embodiments, compound 18, such as a hydroxy-pyrone diene, and compound 19 are mixed in a suitable solvent and heated at a temperature suitable to facilitate formation of phenol 22, such as from 75° C. or less to 200° C. or more, from 100° C. to 175° C., from 125° C. to 160° C. or from 140° C. to 150° C. The solvent may be any solvent suitable to facilitate the reaction, such as an aprotic solvent, for example, an aryl-based solvent, such as 1,2-dichlorobenzene, toluene, or xylene; an alkylnitrile solvent, such as acetonitrile, propionitrile, or butyronitrile; a chlorinated solvent, such as dichloromethane, dichloroethane or chloroform; an ether solvent, such as methyl-tert-butylether; an ester-based solvent, such as ethylpropionate, or ethyl acetate; 1,4-dioxane; or any combination thereof. The reaction may be heated for a time period suitable to facilitate phenol formation, such as from 1 hour or less to 7 days or more, such as from 1 hour to 5 days, from 1 hour to 3 days, from 1 hour to 24 hours, from 3 hours to 24 hours, from 6 hours to 20 hours or from 12 hours to 18 hours, and in certain embodiment, the reaction is heated for 16 hours. In some embodiments, the reaction is monitored by a suitable technique, such as thin layer chromatography, and the reaction is allowed to proceed until substantially all starting materials have reacted. And with respect to Scheme 6, the reaction may be allowed to proceed until substantially all of the intermediate phenol compound 101 as converted to the benzofuranone compound 102. The product typically is isolated from the reaction mixture by a suitable technique known to persons of ordinary skill in the art, such as extraction, column chromatography, or a combination thereof. In certain embodiments, flash column chromatography (FCC) was used.

Compound 18 and/or compounds 19 or 99 may be electronically polarized, and in some embodiments, both compound 18 and compounds 19 or 99 are selected to be electronically polarized, for example, as illustrated in Schemes 5 and 6. When compounds 19 or 99 are selected to be electronically polarized, X may be selected to be both an activating group and a leaving group.

In some embodiments, the reaction may proceed in the presence of a catalyst. The catalyst may be any catalyst that facilitates the Diels-Alder type of reaction. In certain embodiments, the catalyst is a Lewis acid. Suitable Lewis acids include any Lewis acid that facilitates the formation of compounds 22, 101 and/or 102. Exemplary suitable Lewis acids include, but are not limited to, AlCl₃, LiClO₄, SiO₂, FeCl₃, ZnCl₂, Zn(OTf)₂, BF₃, 1-phenyl-3-(2-pyridyl)urea, quinidine, and combinations thereof. In certain embodiments, the Lewis acid is, or comprises, AlCl₃. A Lewis acid may be used in an amount of from greater than zero to 1 molar equivalent (100 mol %) or more, such as from greater than zero to 100 mol %, from 1 mol % to 50 mol %, from 5 mol % to 25 mol %, or from 5 mol % to 15 mol %, and in certain embodiments, 10 mol % is used.

Additionally, or alternatively, the reaction may proceed in the presence of a radical initiator. Suitable radical initiators include any compound that acts as a radical initiator and facilitates the formation of compound 22, such as, but not limited to, butylated hydroxytoluene (BHT), hydroquinone, 2,6-di-tertbutyl phenol, or a combination thereof. The radical initiator may be used in an amount of from greater than zero to 1 molar equivalent (100 mol %) or more, such as from greater than zero to 100 mol %, from 1 mol % to 50 mol %, from 5 mol % to 25 mol %, or from 5 mol % to 15 mol %, and in certain embodiments, 10 mol % is used. In some embodiments, the reaction proceeds in the presence of both a Lewis acid and a radical initiator, and in certain disclosed embodiments, approximately equal amounts of the Lewis acid and the radical initiator are used, such as approximately 10 mol % of each.

And with particular reference to Scheme 6, a protic acid may be used to facilitate ring closure and formation of the benzofuranone. The protic acid may be any protic acid suitable to facilitate the ring closure, such as a haloacetic acid, such as trifluoroacetic acid; an aryl sulfonic acid, such as toluene sulfonic acid; a hydrohalide acid such as hydrochloric acid, hydrobromic acid or hydroiodic acid; or a combination thereof. In certain embodiments, trifluoroacetic acid was used.

### IV. Compounds Made by the Method

Also disclosed herein are compounds made by the method. In some embodiments, the compound has a formula III

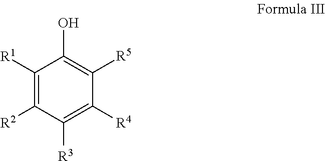

or a salt, or solvate thereof. With respect to Formula III, each of R¹, R², R³, R⁴ and R⁵ independently are as previously defined for Schemes 4 and 5.

In other embodiments, the compound has a structure according to formula IV

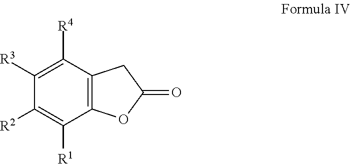

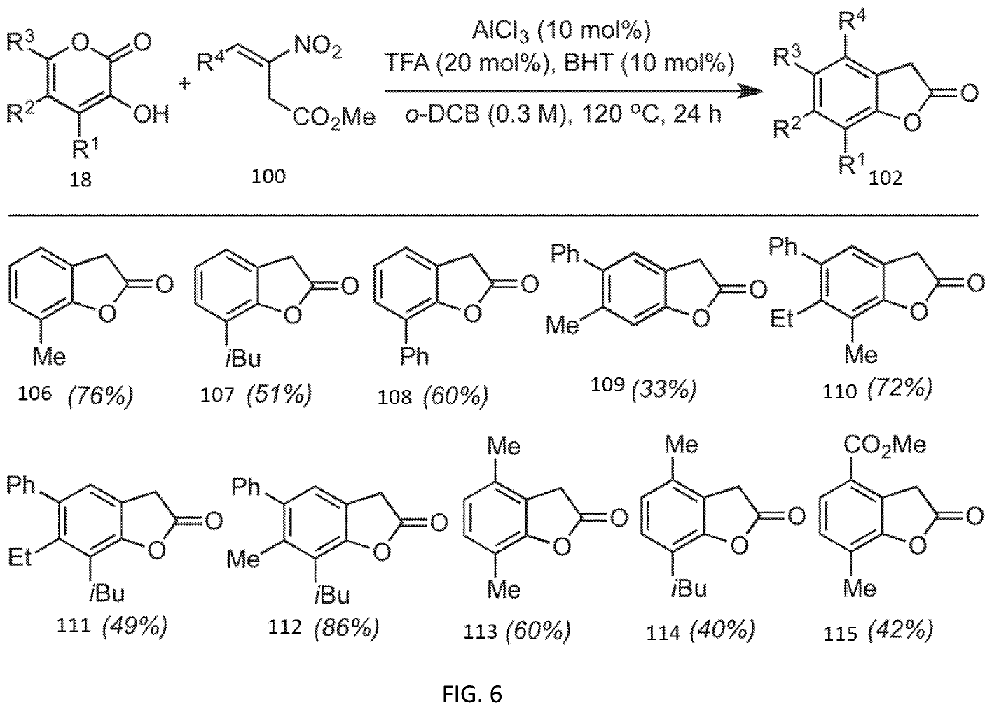

or a salt, or solvate thereof. With respect to Formula IV, R¹, R², R³, and R⁴ are each independently H; aryl; aliphatic, such as alkyl, alkenyl, alkynyl, cycloalkyl, cycloalkenyl, or cycloalkynyl; heterocyclyl, such as heteroaryl or heterocycloaliphatic; alkoxy; heteroaliphatic; or carboxylic ester, such as —CO₂R where R is H, aliphatic or hydroxyalkyl, such as aliphatic or hydroxyalkyl. In some embodiments, each of R¹, R², R³, and R⁴ independently is H, alkyl, cycloalkyl, heteroaryl, heterocycloaliphatic, alkoxy or CO₂R, such as H alkyl, aryl, or CO₂R. And in certain embodiments, each of R¹, R², R³, and R⁴ independently is H or alkyl. And in some embodiments, each of R¹, R², R³, and R⁴ independently are as previously defined for Scheme 6. FIG. 6 provides some exemplary compounds according to formula IV.

In some embodiments of formulas III and IV, at least one of R¹, R², R³, R⁴ and RV, if present, is not H, such as at least 2 of R¹, R², R³, R⁴ and R⁵, at least 3 of R¹, R², R³, R⁴ and R⁵, at least 4 of R¹, R², R³, R⁴ and R⁵, or all 5 of R¹, R², R³, R⁴ and R⁵ are not H. In particular embodiments, at least 3 of R¹, R², R³, R⁴ and R⁵ are not H.

In any embodiments of formulas III and IV, at least 2 of R¹, R², R³, R⁴ and R⁵, if present, are different from each other, such as at least 3 of R¹, R², R³, R⁴ and R⁵, at least 4 of R¹, R², R³, R⁴ and RV, or all 5 of R¹, R², R³, R⁴ and R⁵ are different from each other. In certain embodiments, at least 2 of R¹, R², R³, R⁴ and R⁵, if present, are different from each other, and may be at least 3 of R¹, R², R³, R⁴ and R⁵ are different from each other.

In particular embodiments of formulas III and IV, at least 3 of R¹, R², R³, R⁴ and RV, if present, are not H and at least two of the non-H substituents are different from each other; at least 4 of R¹, R², R³, R⁴ and R⁵ are not H and at least two of the non-H substituents are different from each other; or each of R¹, R², R³, R⁴ and R⁵ are not H and at least two of the substituents are different from each other. And in any embodiment, at least three of the non-H substituents are different from each other, at least four of the non-H substituents are different from each other, or all five substituents are different from each other.

And in certain embodiments, the compound is not 2,6-dimethyl-3-phenylphenol.

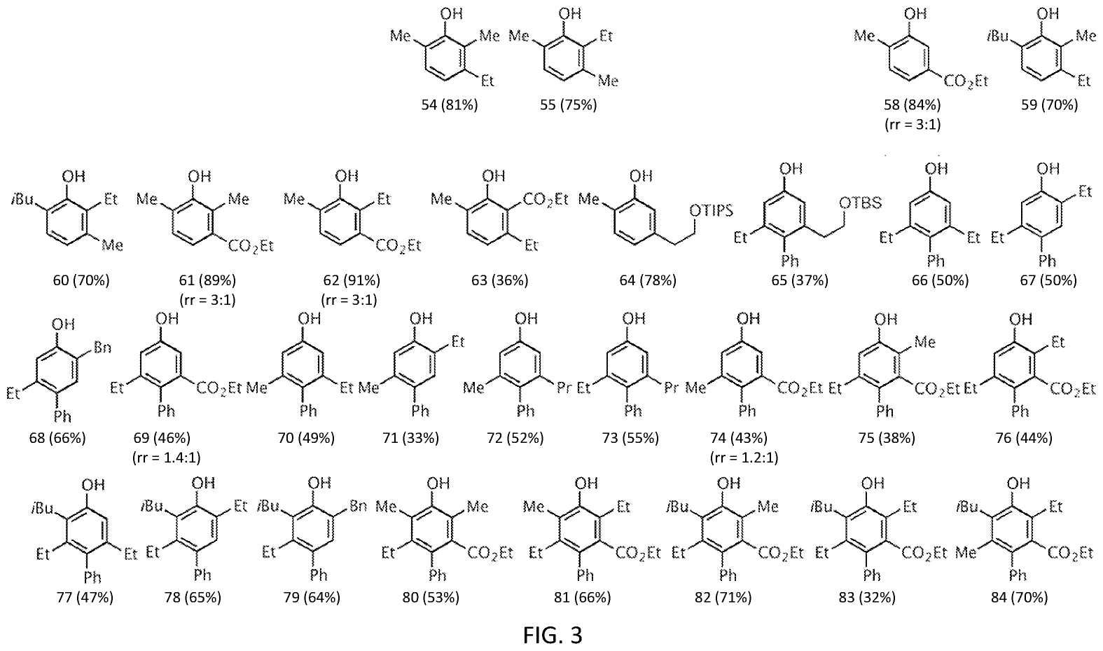

Certain exemplary compounds according to formula III include, but are not limited to, those illustrated in FIG. 3.

In some embodiments of Formula IV, at least 2 of R¹, R², R³, and R⁴ are not H. In certain embodiments, one of the following conditions a), b) or c) applies.

a) If exactly two of R¹, R², R³, and R⁴ are not H then they are different from each other;

b) If exactly three of R¹, R², R³, and R⁴ are not H then at least two of the non-H substituents are different from each other, and may be all three of the non-H substituents are different from each other; or

c) If all four of R¹, R², R³, and R⁴ are not H then at least two of R¹, R², R³, and R⁴ are different from each other, and may be three of R¹, R², R³, and R⁴ are different from each other, and in some embodiments all four of R¹, R², R³, and R⁴ are different from each other.

In other embodiments, if R³ and R⁴ are H, and R² is alkyl, then R¹ is not CO₂H, CO₂alkyl, or -alkyl-CO₂H, or -alkyl-CO₂alkyl, or -alkylNH₂.

In certain embodiments, of formula IV, the compound is not a compound from Table 1 Table 1

TABLE 1
7-acetyl-6-methyl-2-oxo-2,3-dihydrobenzofuran-4,5-dicarboxylic acid;
2,3-Dihydro-6-methyl-2-oxo-4,7-benzofurandicarboxylic acid;
7-Benzoyl-4,6-diphenyl-2(3H)-benzofuranone;
7-Benzoyl-6-(4-methoxyphenyl)-4-phenyl-2(3H)-benzofuranone;
2,3-Dihydro-6-methyl-2-oxo-7-benzofurancarboxaldehyde;
6-methyl-2-oxo-2,3-dihydrobenzofuran-7-carbonitrile;
7-(aminomethyl)-6-methylbenzofuran-2(3H)-one;
7-(2-aminoethyl)-6-methylbenzofuran-2(3H)-one;
2-(6-methyl-2-oxo-2,3-dihydrobenzofuran-7-yl)acetonitrile;
2-(6-methyl-2-oxo-2,3-dihydrobenzofuran-7-yl)acetic acid;
6-methyl-2-oxo-2,3-dihydrobenzofuran-7-carboxylic acid;
2,3-Dihydro-6-methyl-2-oxo-7-benzofuranpropanoic acid;
methyl 2-(6-methyl-2-oxo-2,3-dihydrobenzofuran-7-yl)acetate;
methyl 6-methyl-2-oxo-2,3-dihydrobenzofuran-7-carboxylate;
ethyl 2-(6-methyl-2-oxo-2,3-dihydrobenzofuran-7-yl)acetate;
ethyl 6-methyl-2-oxo-2,3-dihydrobenzofuran-7-carboxylate;
1,1-Dimethylethyl 2,3-dihydro-6-methyl-2-oxo-7-benzofuranpropanoate;
7-(4-Diazo-3-oxobutyl)-6-methyl-2(3H)-benzofuranone;
5-(1,1-Dimethylethyl)-7-methyl-2(3H)-benzofuranone;
7-(1,1-Dimethylethyl)-5-methyl-2(3H)-benzofuranone;
7-(1,1-Dimethylethyl)-5-ethyl-2(3H)-benzofuranone;
7-(1,1-Dimethylethyl)-2,3-dihydro-2-oxo-5-benzofuranacetic acid;
Methyl 2,3-dihydro-5-(1-methylethyl)-2-oxo-7-benzofuranacetate;
7-(1,1-Dimethylethyl)-2,3-dihydro-2-oxo-5-benzofuranpropanoic acid;
Methyl 5-(1,1-dimethylethyl)-2,3-dihydro-2-oxo-7-benzofuranacetate;
methyl 3-(7-(tert-butyl)-2-oxo-2,3-dihydrobenzofuran-5-yl)propanoate;
Octadecyl 7-(1,1-dimethylethyl)-2,3-dihydro-2-oxo-5-benzofuranacetate;
5-Methyl-6-phenyl-2(3H)-benzofuranone;
6-Cyclopentyl-5-methyl-2(3H)-benzofuranone;
6-Cyclohexyl-5-methyl-2(3H)-benzofuranone;
2,3-Dihydro-6-methyl-2-oxo-5-benzofuranpropanoic acid;
Methyl 2,3-dihydro-6-methyl-2-oxo-5-benzofuranpropanoate;
7-Methyl-4-(2-naphthalenyl)-2(3H)-benzofuranone;
2,3-Dihydro-β,4-dimethyl-2-oxo-7-benzofuranpropanoic acid;
4-Methyl-6-(1-methylethyl)-2(3H)-benzofuranone;
Methyl 2,3-dihydro-6-methyl-2-oxo-4-benzofurancarboxylate;
6-methyl-2-oxo-2,3-dihydrobenzofuran-4-carboxylic acid;
2,3-Dihydro-4-methyl-2-oxo-5-benzofuranpropanoic acid;
Methyl 2,3-dihydro-4-methyl-2-oxo-5-benzofuranpropanoate;
Ethyl 2,3-dihydro-4-methyl-2-oxo-5-benzofurancarboxylate;
5-(4-Diazo-3-oxopentyl)-4-methyl-2(3H)-benzofuranone.

### V. Examples

### General Experimental Details

All reactions were carried out under an inert Argon (Ar) atmosphere in oven-dried glassware with magnetic stirring. Flash column chromatography (FCC) was carried out with SiliaFlash P60, 60 Å silica gel. Reactions and column chromatography were monitored with EMD silica gel 60 F254 plates and visualized with potassium permanganate stain.

Reagent grade 1,4-dioxane was dried over calcium hydride and distilled prior to use. 1,2-Dichlorobenzene was distilled under reduce pressure and degassed using three freeze-pump-thaw cycles. All other reagents and solvents were used without further purification from commercial sources. Unless otherwise noted, melting points were obtained from material that solidified after chromatography.

Instrumentation: FT-IR spectra were obtained on NaCl plates with a PerkinElmer Spectrum Vision spectrometer. Microwave reactions were performed with a Biotage® Initiator⁺ Microwave System. Unless otherwise noted, proton and carbon NMR spectra (¹H NMR and ¹³C NMR) were recorded in deuterated chloroform (CDCl₃) on a Bruker 700 MHz Avance III Spectrometer with s carbon-optimized cryoprobe or a Bruker 400 MHz DPX-400 spectrometer. Multiplicities are abbreviated as follows: s=singlet, d=doublet, t=triplet, q=quartet, pent=pentet, sex=sextet, sept=septet, oct=octet, br=broad, m=multiplet. Melting points were determined with a Cole-Parmer instrument and are uncorrected.

### Example 1

### Preparation of Pyrone Coupling Partners

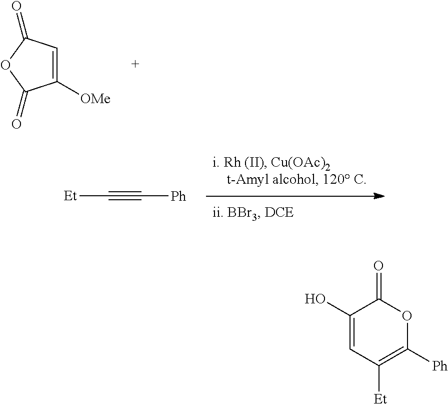 [SMILES: COC1=CC(=O)OC1=O ; CCC#Cc1ccccc1 ; CCc1cc(O)c(=O)oc1-c1ccccc1]

### 5-Ethyl-3-hydroxy-6-phenyl-2H-pyran-2-one (27-S1)

i) To a thick-walled reaction vessel were added a solution of 3-methoxyfuran-2,5-dione (1.98 g, 15.5 mmol, 1.5 eq) in t-amyl alcohol (100 mL), tris(acetonitrile)penta methylcyclopentadienylrhodium(III) hexafluoroantimonate (17 mg, 0.021 mmol, 0.002 eq), Cu(OAc)₂ (2.82 g, 15.5 mmol, 1.5 eq) and 1-phenyl-butyne (1.34 g, 10.3 mmol, 1 eq). The headspace of the vessel was evacuated by brief exposure to vacuum, and the vessel was back-filled with Ar. The vessel was evacuated and back-filled with Ar two additional times. The tube was quickly sealed and heated to 120° C. for 16 hours. The reaction mixture was cooled to room temperature and filtered through Celite with EtOAc to remove solids. The filtrate was concentrated. The residue was purified by FCC (5:1 hexanes:EtOAc) to yield 5-ethyl-3-methoxy-6-phenyl-2H-pyran-2-one as a white solid (1.76 g, 74%).

Data for 5-ethyl-3-methoxy-6-phenyl-2H-pyran-2-one: R_{f}: 0.37 (3:2 hexanes:EtOAc); mp: 111-115° C.; IR (thin film) 1726, 1639 cm⁻¹; ¹H NMR (700 MHz, CDCl₃) δ 7.43 (t, J=7.0 Hz, 2H), 7.38 (t, J=7.7 Hz, 1H), 7.27-7.26 (m, 2H), 6.47 (s, 1H), 3.80 (s, 3H), 2.47 (q, J=7.7 Hz, 2H), 1.20 (t, J=7.0 Hz, 3H); ¹³C NMR (176 MHz, CDCl₃) δ 154.3, 143.5, 136.5, 128.9, 128.8, 127.9, 117.3, 116.5, 56.1, 28.7, 24.1, 12.5; HRMS (ES+) calcd for C₁₄H₁₅O₃ [M+H]: 231.1021, found 231.1015.

ii) To a solution of 5-ethyl-3-methoxy-6-phenyl-2H-pyran-2-one (1.67 g, 7.26 mmol, 1 eq) in 1,2-dichloroethane (73 mL, 0.1 M) was added BBr₃ (1 M in heptane, 11 mL, 11.0 mmol, 1.5 eq) dropwise. The mixture was stirred at room temperature for 20 minutes. The reaction was quenched by addition of saturated aqueous NaHCO₃ (100 mL) and extracted with CH₂Cl₂ (4×30 mL). The combined organic layers were dried over Na₂SO₄, filtered, and concentrated. The residue was purified by FCC (5:1 hexanes:EtOAc) to yield 27-S1 as a light yellow solid.

Data for 27-S1: R_{f}: 0.43 (2:1 hexanes:EtOAc); mp: 113-115° C.; IR (thin film) 3309, 1683, 1651 cm⁻¹; ¹H NMR (700 MHz, CDCl₃) δ 7.48-7.40 (m, 5H), 6.76 (s, 1H), 6.24 (s, 1H), 2.46 (q, J=7.7 Hz, 2H), 1.19 (t, J=7.7 Hz, 3H); ¹³C NMR (176 MHz, CDCl₃) δ 161.3, 148.7, 140.8, 132.2, 129.3, 128.6, 128.4, 118.8, 118.5, 23.4, 14.5; HRMS (ES+) calcd for C₁₃H₁₃O₃ [M+H]: 217.0865, found 217.0868.

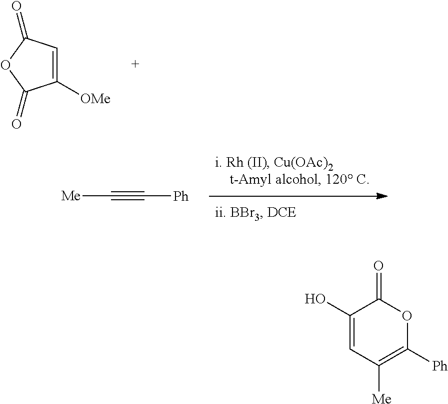 [SMILES: COC1=CC(=O)OC1=O ; CC#Cc1ccccc1 ; Cc1cc(O)c(=O)oc1-c1ccccc1]

### 3-Hydroxy-5-methyl-6-phenyl-2H-pyran-2-one (27-S2)

i) To a thick-walled reaction vessel were added a solution of 3-methoxyfuran-2,5-dione (3.84 g, 30.0 mmol, 1.5 eq) in t-amyl alcohol (200 mL), tris(acetonitrile)penta methylcyclopentadienylrhodium(III) hexafluoroantimonate (33 mg, 0.04 mmol, 0.004 eq), Cu(OAc)₂ (5.46 g, 30.0 mmol, 1.5 eq) and 1-phenyl-propyne (2.32 g, 20.0 mmol, 1 eq). The headspace of the vessel was evacuated by brief exposure to vacuum, and the vessel was back-filled with Ar. The vessel was evacuated and back-filled with Ar two additional times. The tube was quickly sealed and heated to 120° C. for 16 hours. The reaction mixture was cooled to room temperature and filtered through Celite with EtOAc to remove solids. The filtrate was dried over Na₂SO₄, filtered, and concentrated. The residue was purified by FCC (3:1 hexanes:EtOAc) to yield 3-methoxy-5-methyl-6-phenyl-2H-pyran-2-one as a white solid (1.56 g, 36%). Spectroscopic data matched those previously reported.

ii) To a solution of 3-methoxy-5-methyl-6-phenyl-2H-pyran-2-one (1.55 g, 5.5 mmol, 1 eq) in 1,2-dichloroethane (72 mL, 0.1 M) was added BBr₃ (1 M in heptane, 8.6 mL, 8.6 mmol, 1.5 eq) dropwise. The mixture was stirred at room temperature for 20 minutes. The reaction was quenched by addition of saturated aqueous NaHCO₃ (100 mL) and extracted with CH₂Cl₂ (4×30 mL). The combined organic layers were dried over Na₂SO₄, filtered, and concentrated. The residue was purified by FCC (3:1 hexanes:EtOAc) to yield 27-S2 as a white solid (1.37 g, 97%).

Data for 27-S2: R_{f}: 0.54 (pure Et₂O); mp: 134-138° C.; IR (thin film) 3326, 1684, 1649, 1422, 1362 cm⁻¹; ¹H NMR (700 MHz, CDCl₃) δ 7.52 (d, J=7.7 Hz, 2H), 7.44 (t, J=7.7 Hz, 2H), 7.41 (t, J=7.7 Hz, 1H), 6.69 (s, 1H), 5.98 (s, 1H), 2.18 (s, 3H); ¹³C NMR (176 MHz, CDCl₃) δ 161.4, 148.6, 140.3, 132.3, 129.2, 128.6, 128.4, 120.1, 112.7, 17.4; HRMS (ES+) calcd for C₁₂H₁₁O₃ [M+H]: 203.0708, found 203.0709.

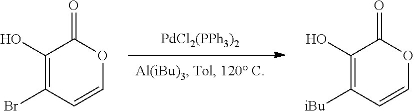

### 3-Hydroxy-4-isobutyl-2H-pyran-2-one (27-S3)

To a thick-walled reaction vessel were added a solution of 4-bromo-3-hydroxy-2H-pyran-2-one (1.20 g, 6.3 mmol, 1 eq) in toluene (13 mL, 0.5M) and PdCl₂(PPh₃)₂ (22 mg, 0.0315 mmol, 0.005 eq). The mixture was stirred for 1 minute, and triisobutyl aluminum (1.1 M in toluene, 6.8 mL, 7.5 mmol, 1.2 eq) was added dropwise. The tube was quickly sealed and heated to 100° C. for 4 hours. The reaction was cooled to room temperature, quenched with 5% citric acid (30 mL), and extracted with EtOAc (5×30 mL). The combined organic layers were dried over Na₂SO₄, filtered, and concentrated. The residue was purified by FCC (5:1 hexanes:EtOAc) to yield 27-S3 as a yellow solid (538.1 mg, 51%).

Data for 27-S3: R_{f}: 0.54 (5:3 hexanes:EtOAc); mp: 39-41° C.; IR (thin film) 3356, 2960, 1756, 1695, 1674 cm⁻¹; ¹H NMR (700 MHz, CDCl₃) δ 7.09 (d, J=4.9 Hz, 1H), 6.32 (s, 1H), 6.09 (d, J=4.9 Hz, 1H), 2.36 (d, J=7.0 Hz, 2H), 1.93 (sept, J=7.0 Hz, 1H), 0.94 (d, J=7.0 Hz, 6H); ¹³C NMR (176 MHz, CDCl₃) δ 161.6, 140.7, 139.1, 130.1, 110.5, 37.6, 27.9, 22.4; HRMS (ES+) calcd for C₉H₁₃O₃ [M+H]: 169.0865, found 169.0863.

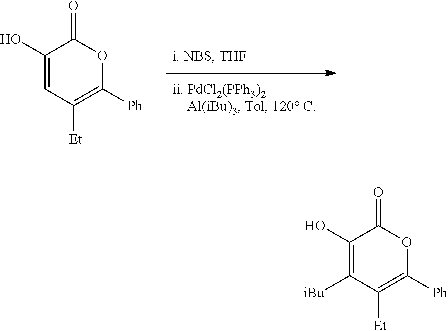

### 5-Ethyl-3-hydroxy-4-isobutyl-6-phenyl-2H-pyran-2-one (27-S4)

i) To a solution of 5-ethyl-3-hydroxy-6-phenyl-2H-pyran-2-one (864 mg, 4.0 mmol, 1 eq) in dimethylformamide (2 mL) was added the solution of N-bromosuccinimide (783 mg, 4.4 mmol, 1.1 eq) in dimethylformamide (2 mL) dropwise. The mixture was stirred at room temperature for 20 minutes. The reaction was quenched with brine (10 mL), and extracted with EtOAc (5×20 mL). The combined organic layers were washed with brine (5×10 mL), dried over Na₂SO₄, filtered, and concentrated. The residue was purified by FCC (5:1 hexanes:EtOAc) to yield 4-bromo-5-ethyl-3-hydroxy-6-phenyl-2H-pyran-2-one as a yellow solid (1.03 g, 87%).

Data for 4-bromo-5-ethyl-3-hydroxy-6-phenyl-2H-pyran-2-one: R_{f}: 0.46 (2:1 hexanes: EtOAc); mp: 150-153° C.; IR (thin film) 3295, 1682, 1371 cm⁻¹; ¹H NMR (700 MHz, CDCl₃) δ 7.47-7.45 (m, 5H), 6.65 (s, 1H), 2.59 (q, J=7.0 Hz, 2H), 1.22 (t, J=7.0 Hz, 3H); ¹³C NMR (176 MHz, CDCl₃) δ 158.6, 148.4, 139.7, 132.2, 129.8, 128.8, 128.5, 119.4, 118.2, 24.0, 14.5; HRMS (ES+) calcd for C₁₃H₂O₃Br [M+H]: 294.9970, found 294.9966.

ii) To a thick-walled reaction vessel were added a solution of 4-bromo-5-ethyl-3-hydroxy-6-phenyl-2H-pyran-2-one (456 mg, 1.55 mmol, 1 eq) in toluene (3.1 mL, 0.5M) and PdCl₂(PPh₃)₂ (11 mg, 0.015 mmol, 0.01 eq). The mixture was stirred for 1 minute, and triisobutyl aluminum (1.1 M in toluene, 1.7 mL, 1.85 mmol, 1.2 eq) was added dropwise. The tube was quickly sealed and heated to 100° C. for 16 hours. The reaction was cooled to room temperature, quenched with 5% citric acid (20 mL), and extracted with EtOAc (5×20 mL). The combined organic layers were dried over Na₂SO₄, filtered, and concentrated. The residue was purified by FCC (5:1 hexanes:EtOAc) to yield 27-S4 as a yellow solid (177 mg, 42%).

Data for 27-S4: R_{f}: 0.40 (4:1 hexanes:EtOAc); mp: 74-78° C.; IR (thin film) 3336, 2959, 1760, 1451 cm⁻¹; ¹H NMR (700 MHz, CDCl₃) δ 7.48-7.46 (m, 2H), 7.44-7.42 (m, 3H), 6.05 (s, 1H), 2.49 (d, J=7.0 Hz, 2H), 2.46 (q, J=7.7 Hz, 2H), 2.02 (sept, J=7.0 Hz, 1H), 1.07 (t, J=7.7 Hz, 3H), 1.0 (d, J=7.0 Hz, 6H); ¹³C NMR (176 MHz, CDCl₃) δ 160.9, 148.5, 138.3, 133.1, 131.9, 129.2, 129.0, 128.4, 120.2, 34.7, 28.5, 22.7, 20.6, 15.4; HRMS (ES+) calcd for C₁₇H₂₁O₃ [M+H]: 273.1491, found 273.1502.

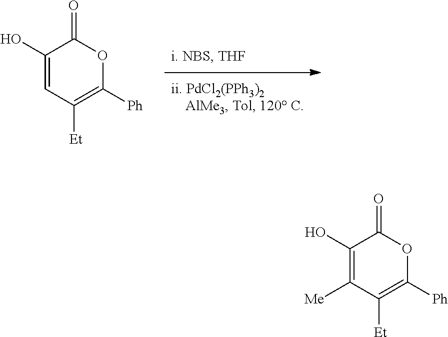 [SMILES: CCc1cc(O)c(=O)oc1-c1ccccc1 ; CCc1c(-c2ccccc2)oc(=O)c(O)c1C]

### 5-Ethyl-3-hydroxy-4-methyl-6-phenyl-2H-pyran-2-one (27-S5)

i) To a solution of 5-ethyl-3-hydroxy-6-phenyl-2H-pyran-2-one (864 mg, 4.0 mmol, 1 eq) in dimethylformamide (2 mL) was added the solution of N-bromosuccinimide (783 mg, 4.4 mmol, 1.1 eq) in dimethylformamide (2 mL) dropwise. The mixture was stirred at room temperature for 20 minutes. The reaction was quenched with brine (10 mL), and extracted with EtOAc (5×20 mL). The combined organic layers were washed with brine (5×10 mL), dried over Na₂SO₄, filtered, and concentrated. The residue was purified by FCC (5:1 hexanes:EtOAc) to yield 4-bromo-5-ethyl-3-hydroxy-6-phenyl-2H-pyran-2-one as a yellow solid (1.03 g, 87%).

ii) To a thick-walled reaction vessel were added a solution of 4-bromo-5-ethyl-3-hydroxy-6-phenyl-2H-pyran-2-one (537 mg, 1.82 mmol, 1 eq) in toluene (3.64 mL, 0.5M) and PdCl₂(PPh₃)₂ (6.4 mg, 0.009 mmol, 0.005 eq). The mixture was stirred for 1 minute, and trimethyl aluminum (25% w/w, 576 mg, 2.0 mmol, 1.1 eq) was added dropwise. The tube was quickly sealed and heated to 100° C. for 3 days. The reaction was cooled to room temperature, quenched with of 5% citric acid (50 mL), and extracted with EtOAc (5×30 mL). The combined organic layers were dried over Na₂SO₄, filtered, and concentrated. The residue was purified by FCC (10:1 hexanes:EtOAc) to yield 27-S5 as a white solid (285 mg, 68%).

Data for 27-S5: R_{f}: 0.57 (3:1 hexanes:EtOAc); mp: 144-146° C.; IR (thin film) 3327, 1680, 1643 cm⁻¹; ¹H NMR (700 MHz, CDCl₃) δ 7.47-7.42 (m, 5H), 6.11 (s, 1H), 2.46 (q, J=7.0 Hz, 2H), 2.21 (s, 3H), 1.15 (t, J=7.0 Hz, 3H); ¹³C NMR (176 MHz, CDCl₃) δ 160.5, 148.4, 138.2, 133.0, 129.3, 128.9, 128.8, 128.4, 120.0, 21.1, 14.9, 11.7; HRMS (ES+) calcd for C₁₄H₁₅O₃ [M+H]: 231.1021, found 231.1017.

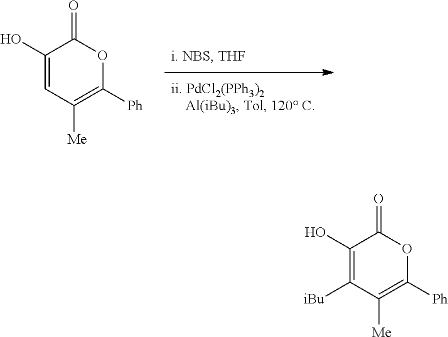

### 3-Hydroxy-4-isobutyl-5-methyl-6-phenyl-2H-pyran-2-one (27-S6)

i) To a solution of 3-hydroxy-5-methyl-6-phenyl-2H-pyran-2-one (1.36 g, 6.73 mmol, 1 eq) in dimethylformamide (3.5 mL) was added a solution of N-bromosuccinimide (1.32 g, 7.4 mmol, 1.1 eq) in dimethylformamide (3.5 mL) dropwise. The mixture was stirred at room temperature for 20 minutes. The reaction was quenched with brine (10 mL), and extracted with EtOAc (5×20 mL). The combined organic layers were washed with brine (4×10 mL), dried over Na₂SO₄, filtered, and concentrated. The residue was purified by FCC (5:1 hexanes:EtOAc) to yield 4-bromo-3-hydroxy-5-methyl-6-phenyl-2H-pyran-2-one as a white solid (1.66 g, 88%).

Data for 4-bromo-3-hydroxy-5-methyl-6-phenyl-2H-pyran-2-one: R_{f}: 0.29 (4:1 hexanes:EtOAc); mp: 190-193° C.; IR (thin film) 3272, 3223, 1682, 1370 cm⁻¹; ¹H NMR (700 MHz, DMSO-d6) δ 10.92 (s, 1H), 7.55-7.49 (m, 5H), 2.15 (s, 1H); ¹³C NMR (176 MHz, DMSO-d6) δ 157.8, 147.0, 140.6, 132.8, 130.0, 129.4, 129.0, 119.9, 113.3, 18.2; HRMS (ES+) calcd for C₁₂H₁₀O₃Br [M+H]: 280.9813, found 280.9812.

ii) To a thick-walled reaction vessel were added a solution of 4-bromo-3-hydroxy-5-methyl-6-phenyl-2H-pyran-2-one (170 mg, 0.6 mmol, 1 eq) in toluene (1.2 mL, 0.5M) and PdCl₂(PPh₃)₂ (4.3 mg, 0.006 mmol, 0.01 eq). The mixture was stirred for 1 minute, and triisobutyl aluminum (1.1 M in toluene, 0.66 mL, 0.73 mmol, 1.2 eq) was added dropwise. The tube was quickly sealed and heated to 100° C. for 4 hours. The reaction was cooled to room temperature, quenched with of 5% citric acid (20 mL), and extracted with EtOAc (5×20 mL). The combined organic layers were dried over Na₂SO₄, filtered, and concentrated. The residue was purified by FCC (5:1 hexanes:EtOAc) to yield 27-S6 as a white solid (83.5 mg, 54%).

Data for 27-S6: R_{f}: 0.49 (3:1 hexanes:EtOAc); mp: 142-144° C.; IR (thin film) 3318, 2959, 1683, 1640 cm⁻¹; ¹H NMR (700 MHz, CDCl₃) δ 7.49-7.48 (m, 2H), 7.45-7.39 (m, 3H), 6.03 (s, 1H), 2.50 (d, J=7.0 Hz, 2H), 2.11 (s, 3H), 1.99 (sept, J=7.0 Hz, 1H), 1.01 (d, J=7.0 Hz, 6H); ¹³C NMR (176 MHz, CDCl₃) δ 161.0, 148.1, 137.9, 133.0, 132.5, 129.2, 128.1, 128.3, 113.8, 35.2, 28.2, 22.7, 14.5; HRMS (ES+) calcd for C₁₆H₁₉O₃ [M+H]: 259.1334, found 259.1335.

### Example 2

### Preparation of Nitroalkane Coupling Partners

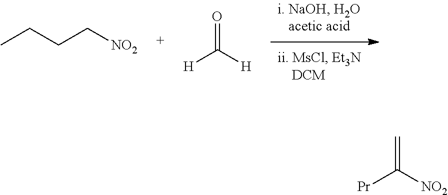

### 2-Nitropent-1-ene (28-S1)

i) To a solution of NaOH (210 mg, 5.25 mmol, 1.05 eq) in water (2 mL) at 0° C. was added 1-nitrobutane (515 mg, 5.00 mmol, 1 eq) dropwise. The mixture was stirred at 0° C. for 10 minutes, followed by room temperature for 1 hour. The flask was cooled to 0° C., and formalin (37% w/w, 426 mg, 5.25 mmol, 1.05 eq) was added dropwise. The mixture was stirred at 0° C. for 2 hours, followed by an additional 16 hours at room temperature. The mixture was cooled to 0° C., and acetic acid (315 mg, 5.5 mmol, 1.1 eq) was added dropwise. The mixture was warmed to room temperature, and extracted with Et₂O (5×20 mL). The combined organic layers were washed with brine, dried over Na₂SO₄, filtered, and concentrated.

ii) To a solution of the crude material (545 mg, 4.1 mmol, 1 eq) from above in CH₂Cl₂ (4.1 mL, 1 M) at 0° C. was added methane sulfonyl chloride (MsCl) (559 mg, 4.9 mmol, 1.5 eq). The mixture was stirred at 0° C. for 10 minutes, upon which time Et₃N (1.66 g, 16.4 mmol, 4 eq) was added dropwise. The mixture was stirred at 0° C. for 1 hour, and then warmed to room temperature for 1 hour. The mixture was quenched with 1 N HCl (30 mL), and extracted with EtOAc (3×30 mL). The combined organic layers were dried over Na₂SO₄, filtered, and concentrated. The residue was purified by FCC (10:1 hexanes:EtOAc) to yield 28-S1 as a yellow oil (113 mg, 24%).

Data for 28-S1: R_{f}: 0.54 (5:1 hexanes:EtOAc); IR (thin film) 2966, 2931, 2878, 1722, 1564, 1528, 1345 cm⁻¹; ¹H NMR (700 MHz, CDCl₃) δ 6.42 (d, J=2.1 Hz, 1H), 5.53 (s, 1H), 2.57 (dt, J=7.7, 0.7 Hz, 2H), 1.57 (sext, J=7.7 Hz, 2H), 0.98 (t, J=7.7 Hz, 1H); ¹³C NMR (176 MHz, CDCl₃) δ 158.1, 117.0, 32.0, 20.4, 13.4; HRMS (ES+) calcd for C₅H₁₀NO₂ [M+H]: 116.0712, found 116.0706.

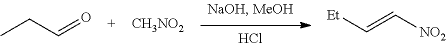

### (E)-1-Nitrobut-1-ene (28-S2)

To a solution of propionaldehyde (5.80 g, 100.0 mmol, 1 eq) in MeOH (50 mL) and nitro methane (24.4 g, 400.0 mmol, 4 eq) at 0° C. was added a solution of 1 N NaOH (250 mL) dropwise. The mixture was stirred at 0° C. for 2 hours. The mixture was then added to a solution of 6 N HCl (60 mL) and stirred at 0° C. for 15 minutes. The mixture was warmed to room temperature, and extracted with EtOAc (4×60 mL). The combined organic layers were dried over Na₂SO₄, filtered and concentrated. The residue was purified by FCC (10:1 hexanes:EtOAc) to yield 28-S2 as an oil (4.36 g, 43%).

Data for 28-S2: R_{f}: 0.6 (4:1 hexanes:EtOAc); IR (thin film) 2979, 2942, 2881, 1650, 1528, 1356 cm⁻¹; ¹H NMR (700 MHz, CDCl₃) δ 7.33 (pent, J=7.0 Hz, 1H), 6.98 (d, J=13.3 Hz, 1H), 2.32 (dpent, J=7.0, 1.4 Hz, 2H), 1.14 (t, J=7.0 Hz, 3H); ¹³C NMR (176 MHz, CDCl₃) δ 144.1, 139.2, 21.9, 11.9; HRMS (ES+) calcd for C₄H₈NO₂ [M+H]: 102.0555, found 102.0558.

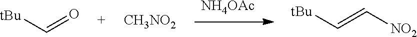

### 3,3-Dimethyl-1-nitrobut-1-ene (28-S3)

To a solution of paraldehyde (2.15 g, 25.0 mmol, 1 eq) in nitro methane (25 mL) was added ammonium acetate (962.5 mg, 12.5 mmol, 0.5 eq). The mixture was heated to reflux for 4.5 hours. The mixture was cooled to room temperature, quenched with brine (100 mL), and extracted with EtOAc (4×30 mL). The combined organic layers were dried over Na₂SO₄, filtered and concentrated. The residue was purified by FCC (25:1 hexanes:EtOAc) to yield 28-S3 as an oil (1.84 g, 57%).

Data for 28-S3: R_{f}: 0.43 (25:1 hexanes:EtOAc); IR (thin film) 2968, 1644, 1530, 1351 cm⁻¹; ¹H NMR (700 MHz, CDCl₃) δ 7.25 (td, J=14.0, 1.4 Hz, 1H), 6.89 (dd, J=14.0, 1.4 Hz, 1H), 1.15-1.14 (m, 9H); ¹³C NMR (176 MHz, CDCl₃) δ 152.1, 137.2, 32.7, 28.5; HRMS (ES+) calcd for C₆H₁₂NO₂ [M+H]: 130.0868, found 130.0869.

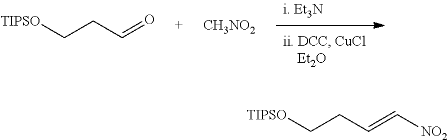

### (E)-Triisopropyl((4-nitrobut-3-en-1-yl)oxy)silane (28-S4)

i) To a solution of 3-((triisopropylsilyl) oxy)propanal (4.6 g, 20.0 mmol, 1 eq) in 1-nitromethane (6.1 g, 100.0 mmol, 5 eq) was added Et₃N (202 mg, 2.0 mmol, 0.1 eq) dropwise.

The mixture was stirred at room temperature for 20 hours. The reaction was quenched with 1 N HCl (20 mL), and extracted with EtOAc (4×30 mL). The combined organic layers were dried over Na₂SO₄, filtered and concentrated.

ii) To a solution of the crude material (1.4 g, 4.8 mmol, 1 eq) from above in Et₂O (16 mL, 0.3 M) was added N,N′-dicyclohexylcarbodiimide (1.09 g 5.28 mmol, 1.1 eq) and CuCl (143 mg, 1.44 mmol, 0.3 eq). The mixture was heated to reflux for 17 hours. The reaction mixture was cooled to room temperature, and filtered through Celite with EtOAc. The filtrate was washed with 1 N HCl (2×30 mL), washed with brine (2×30 mL), dried over Na₂SO₄, filtered and concentrated. The residue was purified by FCC (40:1 hexanes:EtOAc) to yield 28-S4 as an oil (484 mg, 37%, 3:1 E/Z mixture).

Data for 28-S4: R_{f}: 0.54 (5:1 hexanes:EtOAc); IR (thin film) 2944, 2892, 2867, 1528, 1463, 1352 cm⁻¹; ¹H NMR (700 MHz, CDCl₃) δ 7.34 (pent, J=7.0 Hz, 1H) (E), 7.07 (d, J=13.3 Hz, 1H) (E), 6.99 (d, J=8.4 Hz, 0.35H) (Z), 6.38 (q, J=7.0 Hz, 0.35H) (Z), 3.88-3.85 (m, 2.7H) (E/Z), 2.98 (q, J=6.3 Hz, 0.7H) (Z), 2.49 (q, J=6.3 Hz, 2H) (E), 1.14-1.08 (m, 4H) (E/Z), 1.05 (d, J=7.0 Hz, 24H) (E/Z); ¹³C NMR (176 MHz, CDCl₃) (E) δ 140.7, 139.9, 61.1, 32.0, 18.0, 11.9; (Z) δ 139.9, 138.8, 61.3, 31.6, 18.0, 11.9; HRMS (ES+) calcd for C₁₃H₂₇NO₃NaSi [M+Na]: 296.1658, found 296.1667.

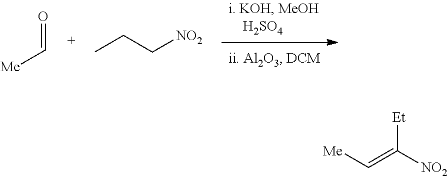

### (E)-3-Nitropent-2-ene (28-S5)

i) To a solution of acetaldehyde (2.64 g, 60.0 mmol, 1 eq) in 1-nitropropane (9.35 g, 105.0 mmol, 1.75 eq) at 0° C. was added 1N KOH in MeOH (6 mL) dropwise. The mixture was stirred at 0° C. for 2.5 hours, upon which time a solution of 0.9 N H₂SO₄ in MeOH (4 mL) was added. The mixture was stirred at 0° C. for 10 minutes, and the solvent was removed under reduced pressure. The residue was dissolved in EtOAc (100 mL), washed sequentially with saturated aqueous NaHCO₃ (30 mL), water (30 mL), and brine (30 mL). The organic layer was dried over Na₂SO₄, filtered and concentrated.

ii) To a solution of the crude material (3.3 g, 24.8 mmol, 1 eq) from above in CH₂Cl₂ (31 mL) was added Al₂O₃ (5.06 g, 49.6 mmol, 2 eq). The mixture was heated to 40° C. for 20 hours. The reaction mixture was cooled to room temperature, and filtered through Celite with CH₂Cl₂ to remove solids. The filtrate was concentrated, and distilled (bp 50-60° C., 7 torr) to yield 28-S5 as an oil (420 mg, 15%).

Data for 28-S5: R_{f}: 0.57 (4:1 hexanes:EtOAc); IR (thin film) 3527, 2980, 1551, 1520, 1335 cm⁻¹; ¹H NMR (700 MHz, CDCl₃) δ 7.12 (q, J=7.7 Hz, 1H), 2.60 (q, J=7.7 Hz, 2H), 1.87 (d, J=7.7 Hz, 3H), 1.09 (t, J=7.7 Hz, 3H); ¹³C NMR (176 MHz, CDCl₃) δ 153.9, 131.0, 19.6, 13.3, 12.3; HRMS (ES+) calcd for C₅H₁₀NO₂ [M+H]: 116.0712, found 116.0712.

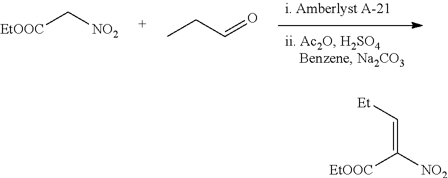

### (E)-Ethyl 2-nitropent-2-enoate (28-S6)

i) To propinaldehyde (870 mg, 15.0 mmol, 3 eq) at 0° C. was added amberlyst A-21 (250 mg). The mixture was stirred at 0° C. for 5 minutes, upon which time ethyl nitroacetate (665 mg, 5.0 mmol, 1 eq) was added dropwise. The mixture was stirred at 0° C. for 2 hours and then warmed to room temperature for 16 hours. The mixture was filtered through Celite with EtOAc to remove solids. The filtrate was concentrated, and the residue was purified by FCC (5:1 hexanes:EtOAc) to yield an alcohol product as oil (845 mg, 88%).

ii) To a solution of the crude alcohol above (611 mg, 3.2 mmol, 1 eq) in acetic anhydride (490 mg, 4.8 mmol, 1.2 eq) was added concentrated H₂SO₄ (0.003 mL). The reaction mixture was heated to 60° C. for 40 minutes. The mixture was diluted with benzene (3.2 mL), and Na₂CO₃ (339 mg, 3.2 mmol, 1 eq) was added. The mixture was maintained at 60° C. until the evolution of CO₂ gas ceased. The mixture was cooled to room temperature, and the solvent was removed under reduced pressure. The residue was purified by FCC (20:1 hexanes:EtOAc) to yield 28-S6 as an oil (204 mg, 37%).

Data for 28-S6: R_{f}: 0.57 (5:1 hexanes:EtOAc); IR (thin film) 2982, 2942, 1736, 1540 cm⁻¹; ¹H NMR (700 MHz, CDCl₃) δ 7.21 (t, J=7.7 Hz, 0.35H) (Z), 6.84 (t, J=7.7 Hz, 1H) (E), 4.34 (q, J=7.0 Hz, 0.7H) (Z), 4.29 (q, J=7.0 Hz, 2H) (E), 2.43 (pent, J=7.7 Hz, 0.7H) (Z), 2.27 (pent, J=7.7 Hz, 2H) (E), 1.33 (t, J=7.0 Hz, 1.05H) (Z), 1.30 (t, J=7.0 Hz, 3H) (E), 1.14 (t, J=7.7 Hz, 1.05H) (Z), 1.69 (t, J=7.7 Hz, 3H) (E); ¹³C NMR (176 MHz, CDCl₃) (E) δ 158.7, 144.5, 141.2, 62.7, 21.6, 14.0, 12.3; (Z) δ 159.8, 144.3, 144.1, 62.6, 21.4, 13.9, 12.5; HRMS (ES+) calcd for C₇H₁₂NO₄ [M+H]: 174.0766, found 174.0760.

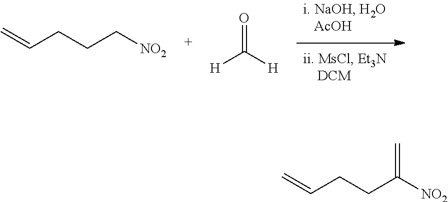

### 2-Nitrohexa-1,5-diene (28-S7)

i) To a solution of NaOH (210 mg, 5.25 mmol, 1.05 eq) in water (2 mL) at 0° C. was added 5-nitropent-1-ene (575 mg, 5.00 mmol, 1 eq) dropwise. The mixture was stirred at 0° C. for 10 minutes, followed by room temperature for 1 hour. The mixture was cooled to 0° C., and formaline (37% w/w, 426 mg, 5.25 mmol, 1.05 eq) was added. The mixture was stirred at 0° C. for 2 hours, followed by an additional 16 hours at room temperature. The mixture was cooled to 0° C., and acetic acid (330 mg, 5.50 mmol, 1.1 eq) was added dropwise. The mixture was warmed to room temperature, and extracted with Et₂O (4×30 mL). The combined organic layers were washed with brine, dried over Na₂SO₄, filtered and concentrated.

ii) To a solution of the crude material (290 mg, 2.0 mmol, 1 eq) from above in CH₂Cl₂ (10 mL) at 0° C. was added MsCl (342 mg, 3.0 mmol, 1.5 eq). The mixture was stirred at 0° C. for 10 minutes, and upon which time Et₃N (808 mg, 8.0 mmol, 4 eq) was added dropwise. The mixture was stirred at 0° C. for 1 hour, and room temperature for 1 hour. The mixture was warmed to room temperature, quenched with 1 N HCl (20 mL), and extracted with EtOAc (4×30 mL). The combined organic layers were dried over Na₂SO₄, filtered and concentrated. The residue was purified by FCC (20:1 hexanes:EtOAc) to yield 28-S7 as an oil (128 mg, 51%).

Data for 28-S7: R_{f}: 0.69 (5:1 hexanes:EtOAc); IR (thin film) 2929, 1714, 1641, 1555, 1526 cm⁻¹; ¹H NMR (700 MHz, CDCl₃) δ 6.46 (s, 1H), 5.79 (dddd, J=16.8, 11.3, 7.0 Hz, 1H), 5.56 (s, 1H), 5.09-5.05 (m, 2H), 2.71 (t, J=7.7 Hz, 2H), 2.32 (q, J=7.0 Hz, 2H); ¹³C NMR (176 MHz, CDCl₃) δ 157.3, 135.9, 117.7, 116.5, 32.1, 29.5; HRMS (ES+) calcd for C₆H₁₀NO₂ [M+H]: 128.0712, found 128.0713.

### Example 3

### Syntheses of Substituted Phenols

### General Procedure for Phenol Syntheses

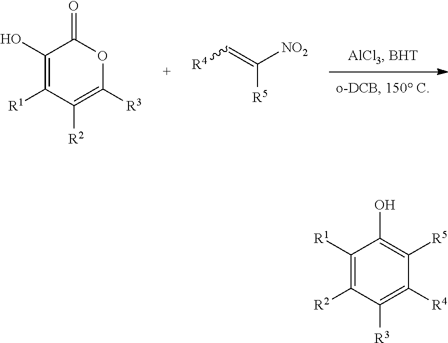

To a thick-walled reaction vessel was added the pyrone, alkene, BHT (0.1 eq) and AlCl₃ (0.1 eq). The vessel was flushed with Ar gas for 5 minutes. 1,2-Dichlorobenzene (0.5 M) was added, and tube was quickly sealed. The reaction mixture was heated to 150° C. for 16 hours unless otherwise noted. The reaction mixture was cooled to room temperature, and the mixture was directly purified by FCC without aqueous work up.

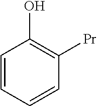

### 2-Propylphenol (30)

3-Hydroxy-2H-pyran-2-one (89.6 mg, 0.8 mmol, 4 eq) and 2-nitropent-1-ene (23 mg, 0.2 mmol, 1 eq) were subjected to the general procedure for 1 hour. Purification by FCC (20:1 hexanes:EtOAc) yielded 30 as a yellow oil (22 mg, 81%). Spectroscopic data for 30 matched those previously reported.

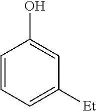

### 3-Ethylphenol (31)

3-Hydroxy-2H-pyran-2-one (89.6 mg, 0.8 mmol, 4 eq) and 1-nitrobut-1-ene (20.2 mg, 0.2 mmol, 1 eq) were subjected to the general procedure. Purification by FCC (10:1 hexanes:EtOAc) yielded 31 as a colorless oil (19.8 mg, 81%). Spectroscopic data for 31 matched those previously reported.

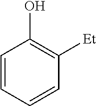

### 2-Ethylphenol (32)

3-Hydroxy-2H-pyran-2-one (89.6 mg, 0.8 mmol, 4 eq) and 2-nitrobut-1-ene (20.2 mg, 0.2 mmol, 1 eq) were subjected to the general procedure for 3 hours. Purification by FCC (15:1 hexanes:EtOAc) yielded 32 as a yellow oil (17.9 mg, 73%). Spectroscopic data for 32 matched those previously reported.

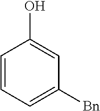 [SMILES: Oc1cccc(Cc2ccccc2)c1]

### 3-Benzylphenol (33)

3-Hydroxy-2H-pyran-2-one (89.6 mg, 0.8 mmol, 4 eq) and (3-nitroallyl)benzene (32.6 mg, 0.2 mmol, 1 eq) were subjected to the general procedure. Purification by FCC (10:1 hexanes:EtOAc) yielded 33 as a colorless oil (23.2 mg, 63%). Spectroscopic data for 33 matched those previously reported.

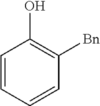 [SMILES: Oc1ccccc1Cc1ccccc1]

### 2-Benzylphenol (34)

3-Hydroxy-2H-pyran-2-one (89.6 mg, 0.8 mmol, 4 eq) and (2-nitroallyl)benzene (32.6 mg, 0.2 mmol, 1 eq) were subjected to the general procedure for 2 hours. Purification by FCC (10:1 hexanes:EtOAc) yielded 34 as a yellow solid (27.5 mg, 74%). Spectroscopic data for 34 matched those previously reported.

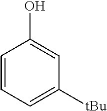 [SMILES: CC(C)(C)c1cccc(O)c1]

### 3-(Tert-butyl)phenol (35)

3-Hydroxy-2H-pyran-2-one (89.6 mg, 0.8 mmol, 4 eq) and 3,3-dimethyl-1-nitrobut-1-ene (25.8 mg, 0.2 mmol, 1 eq) were subjected to the general procedure at 180° C. Purification by FCC (10:1 hexanes:EtOAc) yielded 34 as a colorless oil (25.5 mg, 85%). Spectroscopic data for 35 matched those previously reported.

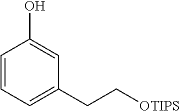

### 3-(2-((Triisopropylsilyl)oxy)ethyl)phenol (36)

3-Hydroxy-2H-pyran-2-one (67.2 mg, 0.6 mmol, 3 eq) and triisopropyl((4-nitrobut-3-en-1-yl)oxy)silane (54.6 mg, 0.2 mmol, 1 eq) were subjected to the general procedure. Purification by FCC (20:1 hexanes:EtOAc) yielded 36 as a yellow oil (32.5 mg, 55%).

Data for 36: R_{f}: 0.49 (5:1 hexanes:EtOAc); IR (thin film) 3344, 2943, 2892, 2866, 1589, 1458 cm⁻¹; ¹H NMR (700 MHz, CDCl₃) δ 7.15 (t, J=7.7 Hz, 1H), 6.80 (d, J=7.7 Hz, 1H), 6.71 (s, 1H), 6.80 (d, J=7.7 Hz, 1H), 4.89 (s, 1H), 3.87 (t, J=7.7 Hz, 2H), 2.82 (t, J=7.7 Hz, 2H), 1.11-1.07 (m, 3H), 1.05 (d, J=7.0 Hz, 18H); ¹³C NMR (176 MHz, CDCl₃) δ 155.4, 141.1, 129.4, 121.7, 116.1, 113.0, 64.8, 39.6, 18.0, 12.0; HRMS (ES+) calcd for C₁₇H₃₀O₂SiNa [M+Na]: 317.1913, found 317.1909.

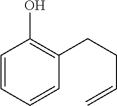

### 2-(But-3-en-1-yl)phenol (37)

3-Hydroxy-2H-pyran-2-one (67.2 mg, 0.6 mmol, 3 eq) and 2-nitrohexa-1,5-diene (25.4 mg, 0.2 mmol, 1 eq) were subjected to the general procedure for 1 hour. Purification by FCC (40:1 hexanes:EtOAc) yielded 37 as a colorless oil (18 mg, 61%). Spectroscopic data for 37 matched those previously reported.

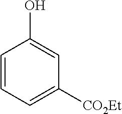 [SMILES: CCOC(=O)c1cccc(O)c1]

### Ethyl 3-hydroxybenzoate (38)

3-Hydroxy-2H-pyran-2-one (89.6 mg, 0.8 mmol, 4 eq) and ethyl 3-nitroacrylate (29.0 mg, 0.2 mmol, 1 eq) were subjected to the general procedure for 10 minutes. Purification by FCC (20:1 hexanes:EtOAc) yielded 38 as a white solid (14.6 mg of 38, 7.1 mg of the corresponding regioisomer, rr=2:1, 65% yield). Spectroscopic data for 38 matched those previously reported.

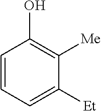

### 3-Ethyl-2-methylphenol (39)

3-Hydroxy-2H-pyran-2-one (89.6 mg, 0.8 mmol, 4 eq) and ethyl 3-nitroacrylate (23.0 mg, 0.2 mmol, 1 eq) were subjected to the general procedure. Purification by FCC (10:1 hexanes:EtOAc) yielded 39 as a yellow solid (19.0 mg, 70%). Spectroscopic data for 39 matched those previously reported.

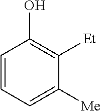

### 2-Ethyl-3-methylphenol (40)

3-Hydroxy-2H-pyran-2-one (89.6 mg, 0.8 mmol, 4 eq) and 3-nitropent-2-ene (23 mg, 0.2 mmol, 1 eq) were subjected to the general procedure. Purification by FCC (20:1 hexaness:EtOAc) yielded 40 as a yellow oil (30.2 mg, 55%).

Data for 40: R_{f}: 0.48 (5:1 hexanes:EtOAc); IR (thin film) 3387, 2967, 1584, 1465 cm-1; H NMR (700 MHz, CDCl₃) δ 6.96 (t, J=7.7 Hz, 1H), 6.75 (d, J=7.7 Hz, 1H), 6.62 (d, J=7.7 Hz, 1H), 4.61 (s, 1H), 2.66 (q, J=7.7 Hz, 2H), 2.31 (s, 3H), 1.15 (t, J=7.7 Hz, 3H); ¹³C NMR (176 MHz, CDCl₃) δ 153.3, 137.6, 128.7, 126.2, 122.8, 112.9, 19.5, 19.3, 13.4; HRMS (ES+) calcd for C₉H₁₃O [M+H]: 137.0966, found 137.0972.

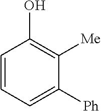 [SMILES: Cc1c(O)cccc1-c1ccccc1]

### 2-Methyl-[1,1′-biphenyl]-3-ol (41)

3-Hydroxy-2H-pyran-2-one (112 mg, 1 mmol, 5 eq) and (2-nitroprop-1-en-1-yl) benzene (32.6 mg, 0.2 mmol, 1 eq) were subjected to the general procedure at 160° C. for 2 days. Purification by FCC (10:1 hexanes:EtOAc) yielded 41 as a yellow oil (26.8 mg, 73%). Spectroscopic data for 41 matched those previously reported.

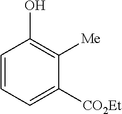 [SMILES: CCOC(=O)c1cccc(O)c1C]

### Ethyl 3-hydroxy-2-methylbenzoate (42)

3-Hydroxy-2H-pyran-2-one (89.6 mg, 0.8 mmol, 4 eq) and ethyl 3-nitrobut-2-enoate (31.8 mg, 0.2 mmol, 1 eq) were subjected to the general procedure at 160° C. Purification by FCC (10:1 hexanes:EtOAc) yielded 42 as a yellow solid (20.9 mg, 58%). Spectroscopic data for 42 matched those previously reported.

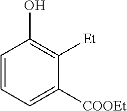 [SMILES: CCOC(=O)c1cccc(O)c1CC]

### Ethyl 2-ethyl-3-hydroxybenzoate (43)

3-Hydroxy-2H-pyran-2-one (67.2 mg, 0.6 mmol, 3 eq) and ethyl 3-nitropent-2-enoate (34.6 mg, 0.2 mmol, 1 eq) were subjected to the general procedure. Purification by FCC (20:1 hexanes:EtOAc) yielded 43 as a yellow solid (15.7 mg of 43, 8.4 mg of 44 rr=2:1, 63% yield).

Data for 43: R_{f}: 0.27 (5:1 hexanes:EtOAc); mp: 67-69° C. IR (thin film) 3406, 2978, 2936, 1693, 1585, 1463, 1287 cm⁻¹; ¹H NMR (700 MHz, CDCl₃) δ 7.37 (d, J=7.7 Hz, 1H), 7.10 (t, J=7.7 Hz, 1H), 6.92 (d, J=7.7 Hz, 1H), 5.26 (s, 1H), 4.36 (q, J=7.0 Hz, 2H), 2.92 (q, J=7.7 Hz, 2H), 1.39 (t, J=7.0 Hz, 3H), 1.23 (t, J=7.7 Hz, 3H); ¹³C NMR (176 MHz, CDCl₃) δ 168.3, 154.0, 132.0, 131.2, 126.4, 122.5, 118.5, 61.1, 20.3, 14.3, 14.3; HRMS (EI+) calcd for C₁₁H₁₄O₃ [M+]: 194.0943, found 194.0938.

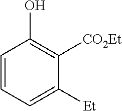 [SMILES: CCOC(=O)c1c(O)cccc1CC]

### Ethyl 2-ethyl-6-hydroxybenzoate (44)

3-Hydroxy-2H-pyran-2-one (89.6 mg, 0.8 mmol, 4 eq) and ethyl 3-nitrobut-2-enoate (34.6 mg, 0.2 mmol, 1 eq) were subjected to the general procedure. Purification by FCC (100:1 hexanes:EtOAc) yielded 44 as a colorless oil (6.3 mg, 16%). Spectroscopic data for 44 matched those previously reported.

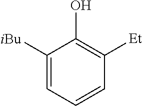

### 2-Ethyl-6-isobutylphenol (45)

3-Hydroxy-4-isobutyl-2H-pyran-2-one (67.2 mg, 0.4 mmol, 2 eq) and 2-nitrobut-1-ene (20.2 mg, 0.2 mmol, 1 eq) were subjected to the general procedure for 15 minutes. Purification by FCC (40:1 hexanes:EtOAc) yielded 45 as a yellow oil (23.6 mg, 66%).

Data for 45: R_{f}: 0.57 (7:1 hexanes:EtOAc); IR (thin film) 3580, 2958, 2931, 2869, 1457, 1188 cm⁻¹; ¹H NMR (700 MHz, CDCl₃) δ 7.01 (dd, J=7.0, 1.4 Hz, 1H), 6.95 (dd, J=7.0, 1.4 Hz, 1H), 6.82 (t, J=7.0 Hz, 1H), 4.63 (s, 1H), 2.64 (q, J=7.7 Hz, 2H), 2.48 (d, J=7.7 Hz, 2H), 1.93 (sept, J=7.0 Hz, 1H), 1.25 (t, J=7.7 Hz, 3H), 0.95 (d, J=7.0 Hz, 6H); ¹³C NMR (176 MHz, CDCl₃) δ 151.5, 129.3, 128.8, 126.8, 126.7, 120.2, 39.5, 28.9, 23.1, 22.7, 14.0; HRMS (EI+) calcd for C₁₂H₁₈O [M+]: 178.1358, found 178.1353.

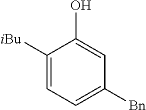

### 5-Benzyl-2-isobutylphenol (46)

3-Hydroxy-4-isobutyl-2H-pyran-2-one (67.2 mg, 0.4 mmol, 2 eq) and (3-nitroallyl) benzene (27.2 mg, 0.2 mmol, 1 eq) were subjected to the general procedure for 4 hours. Purification by FCC (40:1 hexanes:EtOAc) yielded 46 as a yellow oil (24.4 mg, 51%).

Data for 46: R_{f}: 0.15 (5:1 hexanes: CH₂Cl₂); IR (thin film) 3534, 3431, 3027, 2954, 2925, 2867, 1425 cm⁻¹; ¹H NMR (700 MHz, CDCl₃) δ 7.30 (t, J=7.7 Hz, 2H), 7.23-7.20 (m, 3H), 7.00 (d, J=7.7 Hz, 1H), 6.73 (dd, J=7.7, 1.4 Hz, 1H), 6.57 (d, J=1.4 Hz, 1H), 4.58 (s, 1H), 3.92 (s, 2H), 2.45 (d, J=7.0 Hz, 2H), 1.92 (sept, J=7.0 Hz, 1H), 0.94 (d, J=7.0 Hz, 6H); ¹³C NMR (176 MHz, CDCl₃) δ 153.6, 141.1, 140.4, 131.2, 129.0, 128.5, 126.1, 125.1, 121.1, 115.8, 41.5, 39.0, 28.9, 22.6; HRMS (EI+) calcd for C₁₇H₂₀O [M+]: 240.1514, found 240.1513.

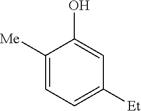

### 2-Methyl-5-ethylphenol (47)

3-Hydroxy-4-methyl-2H-pyran-2-one (75.6 mg, 0.6 mmol, 3 eq) and 1-nitrobut-1-ene (20.2 mg, 0.2 mmol, 1 eq) were subjected to the general procedure for 3 hours. Purification by FCC (15:1 hexanes:EtOAc) yielded 47 as a yellow oil (22.3 mg, 82%). Spectroscopic data for 47 matched those previously reported.

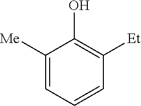

### 2-Ethyl-6-methylphenol (48)

3-Hydroxy-4-methyl-2H-pyran-2-one (50.4 mg, 0.4 mmol, 2 eq) and 2-nitrobut-1-ene (20.2 mg, 0.2 mmol, 1 eq) were subjected to the general procedure for 15 minutes. Purification by FCC (30:1 hexanes:EtOAc) yielded 48 as a yellow oil (19.3 mg, 71%). Spectroscopic data for 48 matched those previously reported.

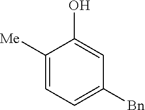 [SMILES: Cc1ccc(Cc2ccccc2)cc1O]

### 5-Benzyl-2-methylphenol (49)

3-Hydroxy-4-methyl-2H-pyran-2-one (50.4 mg, 0.4 mmol, 2 eq) and (3-nitroallyl) benzene (32.6 mg, 0.2 mmol, 1 eq) were subjected to the general procedure. Purification by FCC (40:1 hexanes:EtOAc) yielded 49 as a yellow solid (26.6 mg, 67%).

Data for 49: R_{f}: 0.48 (7:1 hexanes: EtOAc); mp: 34-35° C.; IR (thin film) 3565, 3027, 2957, 2922, 1594, 1470, 1199 cm⁻¹; ¹H NMR (700 MHz, CDCl₃) δ 7.31 (t, J=7.7 Hz, 2H), 7.23-7.20 (m, 3H), 7.06 (d, J=7.7 Hz, 1H), 6.73 (d, J=7.7 Hz, 1H), 6.58 (s, 1H), 4.65 (d, J=2.8 Hz, 1H), 3.92 (s, 2H), 2.23 (s, 3H); ¹³C NMR (176 MHz, CDCl₃) δ 153.8, 141.1, 140.5, 131.0, 129.0, 128.5, 126.1, 121.4, 121.3, 115.5, 41.5, 15.4; HRMS (ES+) calcd for C₁₄H₁₅O [M+H]:199.1123, found 119.1116.

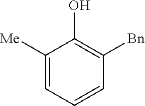 [SMILES: Cc1cccc(Cc2ccccc2)c1O]

### 2-Benzyl-6-methylphenol (50)

3-Hydroxy-4-methyl-2H-pyran-2-one (50.4 mg, 0.4 mmol, 2 eq) and (2-nitroallyl) benzene (32.6 mg, 0.2 mmol, 1 eq) were subjected to the general procedure for 45 minutes. Purification by FCC (20:1 hexanes:EtOAc) yielded 50 as a white solid (31.7 mg, 80%). Spectroscopic data for 50 matched those previously reported.

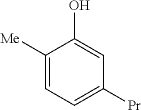

### 2-Methyl-5-propylphenol (51)

3-Hydroxy-4-methyl-2H-pyran-2-one (50.4 mg, 0.4 mmol, 2 eq) and 1-nitropent-1-ene (23 mg, 0.2 mmol, 1 eq) were subjected to the general procedure at 180° C. for 1 hour. Purification by FCC (20:1 hexanes:EtOAc) yielded 51 as a yellow oil (23.2 mg, 77%).

Data for 51: R_{f}: 0.45 (5:1 hexanes: EtOAc); IR (thin film) 3392, 2959, 2929, 2871, 1587, 1456, 1420, 1244 cm⁻¹; ¹H NMR (700 MHz, CDCl₃) δ 7.03 (d, J=7.7 Hz, 1H), 6.69 (dd, J=7.7, 1.4 Hz, 1H), 6.62 (d, J=1.4 Hz, 1H), 4.69 (s, 1H), 2.52 (t, J=7.7 Hz, 2H), 2.23 (s, 3H), 1.63 (sext, J=7.7 Hz, 2H), 0.95 (t, J=7.7 Hz, 3H); ¹³C NMR (176 MHz, CDCl₃) δ 153.6, 142.1, 130.8, 120.9, 120.7, 115.0, 37.6, 24.5, 15.4, 13.9; HRMS (EI+) calcd for C₁₀H₁₄O [M+]: 150.1045, found 150.1051.

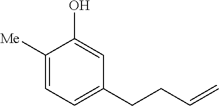

### 5-(But-3-en-1-yl)-2-methylphenol (52)

3-Hydroxy-4-methyl-2H-pyran-2-one (50.4 mg, 0.4 mmol, 2 eq) and 1-nitrohexa-1,5-diene (25.4 mg, 0.2 mmol, 1 eq) were subjected to the general procedure for 6 hours. Purification by FCC (20:1 hexanes:EtOAc) yielded 52 as a yellow oil (19.8 mg, 61%).

Data for 52: R_{f}: 0.43 (5:1 hexanes:EtOAc); IR (thin film) 3400, 2977, 2925, 2856, 1640, 1587, 1419 cm⁻¹; ¹H NMR (700 MHz, CDCl₃) δ 7.03 (d, J=7.0 Hz, 1H), 6.69 (d, J=7.0 Hz, 1H), 6.63 (s, 1H), 5.86 (ddt, J=16.8, 10.5, 6.3 Hz, 1H), 5.47 (d, J=17.5 Hz, 1H), 4.98 (d, J=10.5 Hz, 1H), 4.66 (s, 1H), 2.64 (t, J=7.7 Hz, 2H), 2.35 (q, J=7.7 Hz, 2H), 2.22 (s, 3H); ¹³C NMR (176 MHz, CDCl₃) δ 153.6, 141.2, 138.1, 130.8, 120.9, 120.8, 115.0, 114.9, 35.5, 34.9, 15.4; HRMS (EI+) calcd for C₁₁H₁₄O [M+]: 162.1045, found 162.1042.

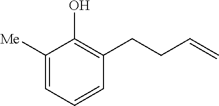

### 2-(But-3-en-1-yl)-6-methylphenol (53)

3-Hydroxy-4-methyl-2H-pyran-2-one (50.4 mg, 0.4 mmol, 2 eq) and 2-nitrohexa-1,5-diene (25.4 mg, 0.2 mmol, 1 eq) were subjected to the general procedure for 15 minutes. Purification by FCC (50:1 hexanes:EtOAc) yielded 53 as a colorless oil (17.1 mg, 53%).

Data for 53: R_{f}: 0.57 (5:1 hexanes:EtOAc); IR (thin film) 3568, 2923, 2854, 1640, 1594, 1468 cm⁻¹; ¹H NMR (700 MHz, CDCl₃) δ 6.99 (d, J=7.7 Hz, 2H), 6.79 (t, J=7.7 Hz, 1H), 5.91 (ddt, J=16.8, 13.3, 7.0 Hz, 1H), 5.09 (dd, J=17.5, 1.4 Hz, 1H), 5.05 (d, J=10.5 Hz, 1H), 4.63 (s, 1H), 2.71 (t, J=7.7 Hz, 2H), 2.38 (q, J=7.7 Hz, 2H), 2.26 (s, 3H); ¹³C NMR (176 MHz, CDCl₃) δ 151.8, 138.3, 128.7, 127.9, 127.2, 123.0, 120.3, 115.2, 33.9, 29.8, 15.9; HRMS (EI+) calcd for C₁₁H₁₄O [M+]: 162.1045, found 162.1044.

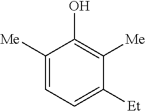 [SMILES: CCc1ccc(C)c(O)c1C]

### 3-Ethyl-2,6-dimethylphenol (54)

3-Hydroxy-4-methyl-2H-pyran-2-one (100 mg, 0.8 mmol, 2 eq) and 2-nitropent-2-ene (23 mg, 0.2 mmol, 1 eq) were subjected to the general procedure. Purification by FCC (10:1 hexanes:EtOAc) yielded 54 as a yellow oil (24.2 mg, 81%).

Data for 54: R_{f}: 0.54 (5:1 hexanes: EtOAc); IR (thin film) 3427, 2960, 2927, 2869, 1493 cm⁻¹; ¹H NMR (700 MHz, CDCl₃) δ 6.92 (d, J=7.7 Hz, 1H), 6.70 (d, J=7.7 Hz, 1H), 4.62 (s, 1H), 2.62 (q, J=7.7 Hz, 2H), 2.23 (s, 3H), 2.21 (s, 3H), 1.19 (t, J=7.7 Hz, 3H); ¹³C NMR (176 MHz, CDCl₃) δ 152.1, 141.7, 127.7, 121.1, 120.2, 120.2, 26.6, 15.9, 15.0, 11.2; HRMS (EI+) calcd for C₁₀H₁₄O [M+]: 150.1045, found 150.1049.

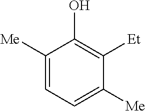 [SMILES: CCc1c(C)ccc(C)c1O]

### 2-Ethyl-3,6-dimethylphenol (55)

3-Hydroxy-4-methyl-2H-pyran-2-one (100 mg, 0.8 mmol, 2 eq) and 3-nitropent-2-ene (23 mg, 0.2 mmol, 1 eq) were subjected to the general procedure. Purification by FCC (15:1 hexanes:EtOAc) yielded 55 as a yellow oil (22.5 mg, 75%).

Data for 55: R_{f}: 0.48 (7:1 hexanes: EtOAc); IR (thin film) 3571, 2966, 2933, 2872, 1464 cm⁻¹; H NMR (700 MHz, CDCl₃) δ 6.87 (d, J=7.7 Hz, 1H), 6.67 (d, J=7.7 Hz, 1H), 4.59 (s, 1H), 2.66 (q, J=7.7 Hz, 2H), 2.28 (s, 3H), 2.22 (s, 3H), 1.15 (t, J=7.7 Hz, 3H); ¹³C NMR (176 MHz, CDCl₃) δ 151.7, 134.9, 128.0, 127.6, 122.1, 120.4, 19.7, 19.2, 15.8, 13.4; HRMS (EI+) calcd for C₁₀H₁₄O [M+]: 150.1045, found 150.1049.

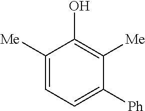 [SMILES: Cc1ccc(-c2ccccc2)c(C)c1O]

### 2,4-Dimethyl-[1,1′-biphenyl]-3-ol (56)

3-Hydroxy-4-methyl-2H-pyran-2-one (75.6 mg, 0.6 mmol, 2 eq) and (2-nitroprop-1-en-1-yl)benzene (32.6 mg, 0.2 mmol, 1 eq) were subjected to the general procedure. Purification by FCC (15:1 hexanes:EtOAc) yielded 56 as a solid (26.5 mg, 67%). Spectroscopic data for 56 matched those previously reported.

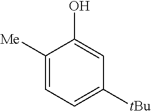 [SMILES: Cc1ccc(C(C)(C)C)cc1O]

### 5-(Tert-butyl)-2-methylphenol (57)

3-Hydroxy-4-methyl-2H-pyran-2-one (50.4 mg, 0.4 mmol, 2 eq) and 3,3-dimethyl-1-nitrobut-1-ene (25.8 mg, 0.2 mmol, 1 eq) were subjected to the general procedure. Purification by FCC (10:1 hexanes:EtOAc) yielded 57 as yellow solid (25.0 mg, 76%). Spectroscopic data for 57 matched those previously reported.

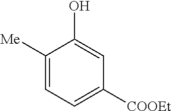 [SMILES: CCOC(=O)c1ccc(C)c(O)c1]

### Ethyl 3-hydroxy-4-methylbenzoate (58)

3-Hydroxy-4-methyl-2H-pyran-2-one (50.4 mg, 0.4 mmol, 2 eq) and ethyl 3-nitroacrylate (29 mg, 0.2 mmol, 1 eq) were subjected to the general procedure for 10 minutes. Purification by FCC (20:1 hexanes:EtOAc) yielded 58 as a yellow solid (22.9 mg, 7.5 mg of the corresponding regioisomer, rr=3:1, 84% yield).

Data for 58: R_{f}: 0.33 (5:1 hexanes: EtOAc); mp: 79-80° C.; IR (thin film) 3431, 2983, 1698 cm⁻¹; ¹H NMR (700 MHz, CDCl₃) δ 7.63 (d, J=1.4 Hz, 1H), 7.52 (dd, J=7.7, 1.4 Hz, 1H), 7.17 (d, J=7.7 Hz, 1H), 6.12 (brs, 1H), 4.37 (q, J=7.0 Hz, 2H), 2.31 (s, 3H), 1.39 (t, J=7.0 Hz, 3H); ¹³C NMR (176 MHz, CDCl₃) δ 167.2, 154.2, 130.9, 130.3, 129.1, 121.7, 115.8, 61.2, 16.2, 14.3; HRMS (ES+) calcd for C₁₀H₁₃O₃ [M+H]: 181.0865, found 181.0870.

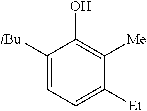

### 2-Ethyl-6-isobutyl-3-methylphenol (59)

3-Hydroxy-4-isobutyl-2H-pyran-2-one (50.4 mg, 0.3 mmol, 3 eq) and 2-nitropent-2-ene (11.5 mg, 0.1 mmol, 1 eq) were subjected to the general procedure. Purification by FCC (15:1 hexanes:EtOAc) yielded 59 as a yellow oil (13.4 mg, 70%) Data for 59: R_{f}: 0.48 (7:1 hexanes:EtOAc); IR (thin film) 3575, 2959, 2931, 2869, 1428 cm⁻¹; ¹H NMR (700 MHz, CDCl₃) δ 6.87 (d, J=7.7 Hz, 1H), 6.71 (d, J=7.7 Hz, 1H), 4.63 (s, 1H), 2.62 (q, J=7.0 Hz, 2H), 2.44 (d, J=7.0 Hz, 2H), 2.20 (s, 3H), 1.90 (sept, J=7.0 Hz, 1H), 1.19 (t, J=7.0 Hz, 3H), 0.95 (d, J=7.0 Hz, 6H); ¹³C NMR (176 MHz, CDCl₃) δ 151.8, 141.5, 128.0, 123.9, 121.2, 120.0, 39.6, 28.9, 26.7, 22.7, 14.8, 11.3; HRMS (EI+) calcd for C₁₃H₂₀O [M+]: 192.1514, found 192.1523.

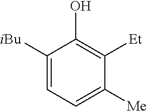

### 3-Ethyl-6-isobutyl-2-methylphenol (60)

3-Hydroxy-4-isobutyl-2H-pyran-2-one (50.4 mg, 0.3 mmol, 3 eq) and 3-nitropent-2-ene (11.5 mg, 0.1 mmol, 1 eq) were subjected to the general procedure. Purification by FCC (50:1 hexanes:EtOAc) yielded 60 as a yellow oil (13.5 mg, 70%).

Data for 60: R_{f}: 0.52 (10:1 hexanes:EtOAc); IR (thin film) 3623, 3583, 2957, 2931, 2867, 1460 cm⁻¹; ¹H NMR (700 MHz, CDCl₃) δ 6.82 (d, J=7.0 Hz, 1H), 6.69 (d, J=7.0 Hz, 1H), 4.63 (s, 1H), 2.66 (q, J=7.0 Hz, 2H), 2.43 (d, J=7.0 Hz, 2H), 2.29 (s, 3H), 1.90 (sept, J=7.0 Hz, 1H), 1.15 (t, J=7.0 Hz, 3H), 0.95 (d, J=7.0 Hz, 6H); ¹³C NMR (176 MHz, CDCl₃) δ 151.5, 134.9, 128.2, 127.9, 124.2, 121.9, 39.6, 29.0, 22.7, 19.8, 19.3, 13.5; HRMS (EI+) calcd for C₁₃H₂₀O [M+]: 192.9926, found 192.9888.

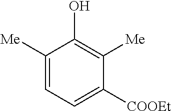 [SMILES: CCOC(=O)c1ccc(C)c(O)c1C]

### Ethyl 3-hydroxy-2,4-dimethylbenzoate (61)

3-Hydroxy-4-methyl-2H-pyran-2-one (189 mg, 1.5 mmol, 1.5 eq) and ethyl 3-nitrobut-2-enoate (159 mg, 1 mmol, 1 eq) were subjected to the general procedure. Purification by FCC (20:1 hexanes:EtOAc) yielded 61 as a yellow solid (128.2 mg of 61, 45.2 mg of the corresponding regioisomer, rr=2.8:1, 89% yield).

Data for 61: R_{f}: 0.42 (5:1 hexanes:EtOAc); mp: 35-37° C.; IR (thin film) 3473, 2980, 2926, 1697 cm⁻¹; ¹H NMR (700 MHz, CDCl₃) δ 7.36 (d, J=7.7 Hz, 1H), 6.99 (d, J=7.7 Hz, 1H), 5.26 (s, 1H), 4.34 (q, J=7.0 Hz, 2H), 2.47 (s, 3H), 2.27 (s, 3H), 1.38 (t, J=7.0 Hz, 3H); ¹³C NMR (176 MHz, CDCl₃) δ 168.3, 152.7, 129.5, 127.7, 127.6, 124.8, 122.3, 60.9, 16.5, 14.3, 12.7; HRMS (ES+) calcd for C₁₁H₁₅O₃ [M+H]: 195.1021, found 195.1018.

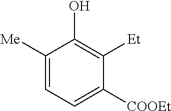 [SMILES: CCOC(=O)c1ccc(C)c(O)c1CC]

### Ethyl 2-ethyl-3-hydroxy-4-methylbenzoate (62)

3-Hydroxy-4-methyl-2H-pyran-2-one (50.4 mg, 0.4 mmol, 2 eq) and ethyl 3-nitropent-2-enoate (34.6 mg, 0.2 mmol, 1 eq) were subjected to the general procedure. Purification by FCC (20:1 hexanes:EtOAc) yielded 62 as a yellow solid (28.5 mg of 62, 9.6 mg of 63, rr=3:1, yield: 91%).

Data for 62: R_{f}: 0.30 (5:1 hexanes:EtOAc); mp: 38-40° C.; IR (thin film) 3486, 2978, 2936, 2875, 1699, 1271 cm⁻¹; ¹H NMR (700 MHz, CDCl₃) δ 7.34 (d, J=7.7 Hz, 1H), 7.01 (d, J=7.7 Hz, 1H), 5.02 (s, 1H), 4.34 (q, J=7.0 Hz, 2H), 2.95 (q, J=7.0 Hz, 2H), 2.28 (s, 3H), 1.38 (t, J=7.0 Hz, 3H), 1.23 (t, J=7.0 Hz, 3H); ¹³C NMR (176 MHz, CDCl₃) δ 168.1, 152.3, 130.6, 129.3, 127.8, 127.5, 122.4, 60.8, 20.4, 16.4, 14.3; HRMS (ES+) calcd for C₁₂H₁₇O₃ [M+H]: 209.1178, found 209.1178.

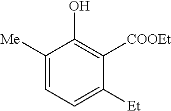 [SMILES: CCOC(=O)c1c(CC)ccc(C)c1O]

### Ethyl 6-ethyl-2-hydroxy-3-methylbenzoate (63)

3-Hydroxy-4-methyl-2H-pyran-2-one (50.4 mg, 0.4 mmol, 2 eq) and ethyl 2-nitropent-2-enoate (34.6 mg, 0.2 mmol, 1 eq) were subjected to the general procedure for 7 hours.

Purification by FCC (20:1 hexanes:EtOAc) yielded 63 as a yellow oil (15 mg, 36%), Data for 63: R_{f}: 0.75 (5:1 hexanes:EtOAc); IR (thin film) 2978, 2932, 1655, 1248 cm-1; H NMR (700 MHz, CDCl₃) δ 11.51 (d, J=0.7 Hz, 1H), 7.18 (d, J=7.7 Hz, 1H), 6.65 (d, J=7.7 Hz, 1H), 4.44 (q, J=7.0 Hz, 2H), 2.91 (q, J=7.0 Hz, 2H), 2.22 (s, 3H), 1.44 (t, J=7.0 Hz, 3H), 1.20 (t, J=7.0 Hz, 3H); ¹³C NMR (176 MHz, CDCl₃) δ 172.0, 160.9, 144.8, 135.1, 124.2, 120.9, 111.2, 61.6, 29.6, 16.4, 15.9, 14.0; HRMS (ES+) calcd for C₁₂H₁₇O₃ [M+H]: 209.1178, found 209.1182.

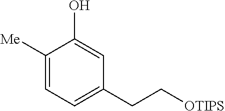

### 2-Methyl-5-(2-((triisopropylsilyl)oxy)ethyl)phenol (64)

3-Hydroxy-4-methyl-2H-pyran-2-one (50.4 mg, 0.4 mmol, 2 eq) and triisopropyl((4-nitrobut-3-en-1-yl)oxy)silane (54.6 mg, 0.2 mmol, 1 eq) were subjected to the general procedure. Purification by FCC (20:1 hexanes:EtOAc) yielded 64 as a yellow oil (48.2 mg, 78%).

Data for 64: R_{f}: 0.56 (5:1 hexanes:EtOAc); IR (thin film) 3384, 2953, 2892, 2866, 1462 cm⁻¹; ¹H NMR (700 MHz, CDCl₃) δ 7.03 (d, J=7.7 Hz, 1H), 6.71 (d, J=7.7 Hz, 1H), 6.65 (s, 1H), 4.76 (s, 1H), 3.85 (t, J=7.7 Hz, 2H), 2.80 (t, J=7.7 Hz, 2H), 2.22 (s, 3H), 1.13-1.07 (m, 3H), 1.06 (d, J=6.3 Hz, 18H); ¹³C NMR (176 MHz, CDCl₃) δ 153.6, 138.3, 130.8, 121.4, 121.3, 115.7, 65.0, 39.3, 18.0, 15.4, 12.0; HRMS (ES+) calcd for C₁₈H₃₂O₂SiNa [M+Na]: 331.2069, found 331.2066.

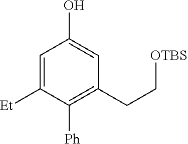 [SMILES: CCc1cc(O)cc(CCO[Si](C)(C)C(C)(C)C)c1-c1ccccc1]

### 2-(2-((Tert-butyldimethylsilyl)oxy)ethyl)-6-ethyl-[1,1′-biphenyl]-4-ol (65)

5-Ethyl-3-hydroxy-6-phenyl-2H-pyran-2-one (21.6 mg, 0.1 mmol, 1 eq) and tert-butyldimethyl ((4-nitrobut-3-en-1-yl)oxy)silane (46.2 mg, 0.2 mmol, 2 eq) were subjected to the general procedure. Purification by FCC (20:1 hexanes:EtOAc) yielded 65 as a yellow solid (13.2 mg, 37%).

Data for 65: R_{f}: 0.42 (5:1 hexanes:EtOAc); mp: 74-78° C.; IR (thin film) 3339, 2957, 2929, 2857, 1609, 1456 cm⁻¹; ¹H NMR (700 MHz, CDCl₃) δ 7.39 (t, J=7.0 Hz, 2H), 7.33 (t, J=7.0 Hz, 1H), 7.14 (d, J=7.0 Hz, 2H), 6.64 (d, J=2.8 Hz, 2H), 4.76 (s, 1H), 3.56 (t, J=7.7 Hz, 2H), 2.53 (t, J=7.7 Hz, 2H), 2.27 (q, J=7.7 Hz, 2H), 1.00 (t, J=7.7 Hz, 3H), 0.81 (s, 9H), −0.13 (s, 6H); ¹³C NMR (176 MHz, CDCl₃) δ 154.5, 144.3, 139.9, 138.3, 134.3, 130.1, 128.2, 126.7, 114.0, 112.9, 64.1, 37.4, 26.9, 26.0, 18.3, 15.4, −5.4; HRMS (ES+) calcd for C₂₂H₃₂O₂SiNa [M+Na]: 379.2069, found 379.2063.

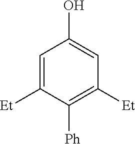 [SMILES: CCc1cc(O)cc(CC)c1-c1ccccc1]

### 2,6-Diethyl-[1,1′-biphenyl]-4-ol (66)

5-Ethyl-3-hydroxy-6-phenyl-2H-pyran-2-one (21.6 mg, 0.1 mmol, 1 eq) and 1-nitrobut-1-ene (80.8 mg, 0.8 mmol, 8 eq) were subjected to the general procedure with hydroquinone (1.1 mg, 0.01 mmol, 0.1 eq) added as an additional radical inhibitor. Purification by FCC (20:1 hexanes:EtOAc) yielded 66 as a yellow solid (11.2 mg, 50%).

Data for 66: R_{f}: 0.36 (5:1 hexanes:EtOAc); mp: 99-101° C.; IR (thin film) 3357, 2965, 2933, 2871, 1609, 1454 cm⁻¹; ¹H NMR (700 MHz, CDCl₃) δ 7.40 (dt, J=7.7, 1.4 Hz, 2H), 7.34 (tt, J=7.7, 1.4 Hz, 1H), 7.16 (dd, J=7.7, 1.4 Hz, 2H), 6.64 (s, 2H), 4.78 (s, 2H), 2.29 (q, J=7.7 Hz, 4H), 1.02 (t, J=7.7 Hz, 6H); ¹³C NMR (176 MHz, CDCl₃) δ 154.7, 144.1, 140.2, 133.7, 130.1, 128.0, 126.6, 112.3, 26.9, 15.4; HRMS (EI+) calcd for C₁₆H₁₅O [M+]: 226.1358, found 226.1365.

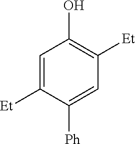 [SMILES: CCc1cc(-c2ccccc2)c(CC)cc1O]

### 2,5-Diethyl-[1,1′-biphenyl]-4-ol (67)

5-Ethyl-3-hydroxy-6-phenyl-2H-pyran-2-one (43.2 mg, 0.2 mmol, 1 eq) and 2-nitrobut-1-ene (60.6 mg, 0.6 mmol, 3 eq) were subjected to the general procedure at 180° C. for 7 hours. Purification by FCC (20:1 hexanes:EtOAc) yielded 67 as a yellow solid (22.5 mg, 50%).

Data for 67: R_{f}: 0.39 (5:1 hexanes:EtOAc); mp: 65-67° C.; IR (thin film) 3400, 2965, 2930, 2872, 1615, 1488 cm⁻¹; ¹H NMR (700 MHz, CDCl₃) δ 7.40 (t, J=7.0 Hz, 2H), 7.34-7.31 (m, 3H), 7.01 (s, 1H), 6.74 (s, 1H), 4.74 (s, 1H), 2.66 (q, J=7.7 Hz, 2H), 2.55 (q, J=7.7 Hz, 2H), 1.27 (t, J=7.7 Hz, 3H), 1.10 (t, J=7.7 Hz, 3H); ¹³C NMR (176 MHz, CDCl₃) δ 152.7, 141.9, 140.5, 134.4, 130.9, 129.5, 128.0, 126.9, 126.5, 115.0, 25.8, 22.6, 15.6, 14.1; HRMS (EI+) calcd for C₁₆H₁₈O [M+]: 226.1358, found 226.1369.

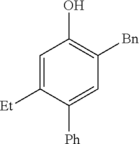 [SMILES: CCc1cc(O)c(Cc2ccccc2)cc1-c1ccccc1]

### 5-Benzyl-2-ethyl-[1,1′-biphenyl]-4-ol (68)

5-Ethyl-3-hydroxy-6-phenyl-2H-pyran-2-one (86.4 mg, 0.4 mmol, 2 eq) and (2-nitroallyl) benzene (32.6 mg, 0.2 mmol, 1 eq) were subjected to the general procedure for 6 hours. Purification by FCC (10:1 hexanes:EtOAc) yielded 68 as a yellow solid (38.2 mg, 66%).

Data for 68: R_{f}: 0.39 (5:1 hexanes:EtOAc); mp: 93-97° C.; IR (thin film) 3534, 3027, 2966, 2930, 1614, 1488 cm⁻¹; ¹H NMR (700 MHz, CDCl₃) δ 7.40 (dt, J=7.0, 1.4 Hz, 2H), 7.34 (tt, J=7.0, 1.4 Hz, 1H), 7.32-7.29 (m, 6H), 7.23 (tt, J=7.7, 1.4 Hz, 1H), 7.03 (s, 1H), 6.76 (s, 1H), 4.73 (s, 1H), 4.02 (s, 2H), 2.57 (q, J=7.7 Hz, 2H), 1.12 (t, J=7.7 Hz, 3H); ¹³C NMR (176 MHz, CDCl₃) δ 153.1, 141.7, 141.4, 140.0, 134.5, 132.6, 129.6, 128.7, 128.7, 128.0, 126.5, 126.4, 124.1, 115.7, 36.2, 25.9, 15.6; HRMS (EI+) calcd for C₂₁H₂₀O [M+]: 288.1514, found 288.1526.

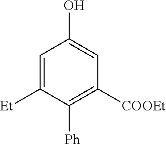 [SMILES: CCOC(=O)c1cc(O)cc(CC)c1-c1ccccc1]

### Ethyl 6-ethyl-4-hydroxy-[1,1′-biphenyl]-2-carboxylate (69)

5-Ethyl-3-hydroxy-6-phenyl-2H-pyran-2-one (43.2 mg, 0.2 mmol, 1 eq) and 1-nitropent-1-ene (58 mg, 0.4 mmol, 2 eq) were subjected to the general procedure. Purification by FCC (20:1 hexanes:EtOAc) yielded 69 as a yellow solid (14.1 mg of 69, 10.5 mg of the corresponding regioisomer, rr=1.4:1, 46% yield).

Data for 69: R_{f}: 0.33 (5:1 hexanes:EtOAc); mp: 101-103° C.; IR (thin film) 3386, 2970, 2933, 1703, 1609 cm⁻¹; ¹H NMR (700 MHz, CDCl₃) δ 7.35 (tt, J=7.0, 1.4 Hz, 2H), 7.32 (tt, J=7.0, 1.4 Hz, 1H), 7.17 (d, J=2.7 Hz, 1H), 7.16 (td, J=7.0, 1.4 Hz, 2H), 6.93 (d, J=2.8 Hz, 1H), 5.32 (brs, 1H), 3.95 (q, J=7.0 Hz, 2H), 2.38 (q, J=7.7 Hz, 2H), 1.02 (t, J=7.0 Hz, 3H), 0.88 (t, J=7.7 Hz, 3H); ¹³C NMR (176 MHz, CDCl₃) δ 168.7, 154.6, 145.2, 139.8, 133.8, 133.3, 129.5, 127.7, 126.7, 118.3, 113.6, 60.9, 26.5, 15.3, 13.5; HRMS (ES+) calcd for C₁₇H₁₉O₃ [M+H]: 271.1334, found 271.1322.

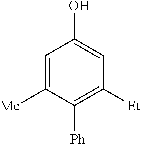 [SMILES: CCc1cc(O)cc(C)c1-c1ccccc1]

### 2-Ethyl-6-methyl-[1,1′-biphenyl]-4-ol (70)

3-Hydroxy-5-methyl-6-phenyl-2H-pyran-2-one (20.2 mg, 0.1 mmol, 1 eq) and 1-nitrobut-1-ene (50.5 mg, 0.5 mmol, 5 eq) were subjected to the general procedure, except 2,6-di-tertbutyl phenol (10.3 mg, 0.05 mmol, 0.5 eq) was used as a radical inhibitor instead of BHT. Purification by FCC (20:1 hexanes:EtOAc) yielded 70 as a yellow solid (10.4 mg, 49%).

Data for 70: R_{f}: 0.45 (5:1 hexanes:EtOAc); mp: 89-93° C.; IR (thin film) 3306, 2968, 2926, 1593, 1460, 1299 cm⁻¹; ¹H NMR (700 MHz, CDCl₃) δ 7.40 (t, J=7.7 Hz, 2H), 7.33 (t, J=7.7 Hz, 1H), 7.14 (d, J=7.7 Hz, 2H), 6.63 (d, J=2.1 Hz, 1H), 6.60 (d, J=2.1 Hz, 1H), 4.65 (s, 1H), 2.30 (q, J=7.7 Hz, 2H), 1.97 (s, 3H), 1.02 (t, J=7.7 Hz, 3H); ¹³C NMR (176 MHz, CDCl₃) δ 154.4, 144.0, 140.5, 137.9, 134.2, 129.9, 128.3, 126.5, 113.9, 112.2, 26.8, 21.0, 15.4; HRMS (ES+) calcd for C₁₅H₁₇O [M+H]:213.1279, found 213.1274.

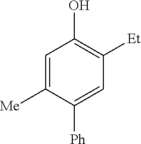 [SMILES: CCc1cc(-c2ccccc2)c(C)cc1O]

### 5-Ethyl-2-methyl-[1,1′-biphenyl]-4-ol (71)

3-Hydroxy-5-methyl-6-phenyl-2H-pyran-2-one (20.2 mg, 0.1 mmol, 1 eq) and 2-nitrobut-1-ene (50.5 mg, 0.5 mmol, 5 eq) were subjected to the general procedure, except 2,6-di-tertbutyl phenol (10.3 mg, 0.05 mmol, 0.5 eq) was used as a radical inhibitor instead of BHT. Purification by FCC (20:1 hexanes:EtOAc) yielded 71 as a yellow solid (9.5 mg, 45%).

Data for 71: R_{f}: 0.40 (5:1 hexanes:EtOAc); mp: 56-56° C.; IR (thin film) 3406, 2965, 2929, 1615 cm⁻¹; ¹H NMR (700 MHz, CDCl₃) δ 7.40 (t, J=7.7 Hz, 2H), 7.33-7.31 (m, 3H), 7.03 (s, 1H), 6.70 (s, 1H), 4.69 (s, 1H), 2.65 (q, J=7.7 Hz, 2H), 2.22 (s, 3H), 1.26 (t, J=7.7 Hz, 3H); ¹³C NMR (176 MHz, CDCl₃) δ 152.4, 141.8, 134.7, 134.1, 130.9, 129.5, 128.0, 127.1, 126.4, 116.8, 22.6, 20.1, 14.2; HRMS (ES+) calcd for C₁₅H₁₇O [M+H]:213.1255, found 213.1260.

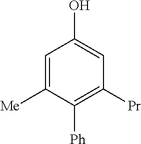

### 2-Methyl-6-propyl-[1,1′-biphenyl]-4-ol (72)

3-Hydroxy-5-methyl-6-phenyl-2H-pyran-2-one (20.2 mg, 0.1 mmol, 1 eq) and 1-nitropent-1-ene (58 mg, 0.5 mmol, 5 eq) were subjected to the general procedure, except 2,6-di-tertbutyl phenol (10.3 mg, 0.05 mmol 0.5 eq) was used as a radical inhibitor instead of BHT. Purification by FCC (20:1 hexanes:EtOAc) yielded 72 as a yellow solid (11.7 mg, 55%).

Data for 72: R_{f}: 0.39 (5:1 hexanes:EtOAc); mp: 51-54° C.; IR (thin film) 3338, 2959, 2929, 2870, 1608, 1592, 1455, 1307, 1151 cm⁻¹; ¹H NMR (700 MHz, CDCl₃) δ 7.40 (t, J=7.7 Hz, 2H), 7.33 (tt, J=7.7, 1.4 Hz, 1H), 7.13 (dd, J=1.4, 7.7 Hz, 2H), 6.62 (d, J=2.8 Hz, 1H), 6.61 (d, J=2.1 Hz, 1H), 4.77 (s, 1H), 2.26 (t, J=7.7 Hz, 2H), 1.97 (s, 3H), 1.43 (sext, J=7.7 Hz, 2H), 0.77 (t, J=7.7 Hz, 3H); ¹³C NMR (176 MHz, CDCl₃) δ 154.2, 142.4, 140.6, 137.9, 134.5, 129.9, 128.2, 126.5, 114.0, 112.9, 35.7, 24.2, 21.1, 14.1; HRMS (EI+) calcd for C₁₆H₁₈O [M+]: 226.1358, found 226.1354.

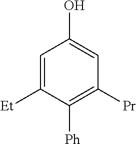

### 2-Ethyl-6-propyl-[1,1′-biphenyl]-4-ol (73)

5-Ethyl-3-hydroxy-6-phenyl-2H-pyran-2-one (21.6 mg, 0.1 mmol, 1 eq) and 1-nitropent-1-ene (58 mg, 0.5 mmol, 5 eq) were subjected to the general procedure. Purification by FCC (20:1 hexanes:EtOAc) yielded 73 as a yellow solid (13.2 mg, 55%).

Data for 73: R_{f}: 0.45 (5:1 hexanes:EtOAc); mp: 73-75° C.; IR (thin film) 3306, 2960, 2929, 2869, 1593, 1455 cm⁻¹; ¹H NMR (400 MHz, CDCl₃) δ 7.41-7.37 (m, 2H), 7.33 (tt, J=6.4, 1.6 Hz, 1H), 7.14 (tt, J=6.4, 1.6 Hz, 2H), 6.63 (d, J=2.8 Hz, 1H), 6.61 (d, J=2.8 Hz, 1H), 4.68 (s, 1H), 2.31-2.21 (m, 4H), 1.42 (sext, J=7.2 Hz, 2H), 1.01 (t, J=7.6 Hz, 3H), 0.76 (t, J=7.2 Hz, 3H); ¹³C NMR (100 MHz, CDCl₃) δ 154.5, 144.1, 142.5, 140.2, 134.0, 130.2, 128.0, 126.5, 112.9, 112.3, 35.8, 26.9, 24.2, 15.3, 14.0; HRMS (ES+) calcd for C₁₇H₂₁O [M+H]: 241.1592, found 241.1599.

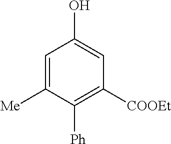 [SMILES: CCOC(=O)c1cc(O)cc(C)c1-c1ccccc1]

### Ethyl 4-hydroxy-6-methyl-[1,1′-biphenyl]-2-carboxylate (74)

3-Hydroxy-5-methyl-6-phenyl-2H-pyran-2-one (40.4 mg, 0.2 mmol, 1 eq) and ethyl 3-nitroacrylate (58 mg, 0.4 mmol, 2 eq) were subjected to the general procedure. Purification by FCC (20:1 hexanes:EtOAc) yielded 74 as a yellow solid (12 mg of 74, 10 mg of the corresponding regioisomer, rr=1.2:1, 43% yield).

Data for 74: R_{f}: 0.24 (5:1 hexanes:EtOAc); mp: 117-121° C.; IR (thin film) 3386, 2981, 2925, 1705, 1611, 1452, 1317 cm⁻¹; ¹H NMR (700 MHz, CDCl₃) δ 7.36 (t, J=7.7 Hz, 2H), 7.31 (tt, J=7.7, 1.4 Hz, 1H), 7.19 (d, J=2.8 Hz, 1H), 7.15-7.13 (m, 2H), 6.90 (d, J=2.8 Hz, 1H), 3.95 (q, J=7.0 Hz, 2H), 2.06 (s, 3H), 0.88 (t, J=7.0 Hz, 3H); ¹³C NMR (176 MHz, CDCl₃) δ 168.7, 154.4, 140.2, 139.1, 134.4, 133.0, 129.2, 127.9, 126.7, 120.0, 113.7, 60.9, 20.9, 13.5; HRMS (ES+) calcd for C₁₆H₁₆O₃Na [M+Na]: 279.0997, found 279.0922.

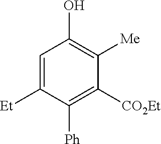 [SMILES: CCOC(=O)c1c(C)c(O)cc(CC)c1-c1ccccc1]

### Ethyl 6-ethyl-4-hydroxy-3-methyl-[1,1′-biphenyl]-2-carboxylate (75)

5-Ethyl-3-hydroxy-6-phenyl-2H-pyran-2-one (86.4 mg, 0.4 mmol, 2 eq) and ethyl 3-nitrobut-2-enoate (31.8 mg, 0.2 mmol, 1 eq) were subjected to the general procedure for 5 days. Purification by FCC (10:1 hexanes:EtOAc) yielded 75 as a yellow solid (21.6 mg, 38%).

Data for 75: R_{f}: 0.42 (5:1 hexanes:EtOAc); mp: 91-95° C.; IR (thin film) 3396, 2968, 1699, 1600, 1463, 1309 cm⁻¹; ¹H NMR (700 MHz, CDCl₃) δ 7.35 (t, J=7.7 Hz, 2H), 7.30 (tt, J=7.7, 1.4 Hz, 1H), 7.21 (dd, J=7.7, 1.4 Hz, 2H), 6.75 (s, 1H), 5.29 (s, 1H), 3.93 (q, J=7.0 Hz, 2H), 2.34 (q, J=7.7 Hz, 2H), 2.20 (s, 3H), 0.99 (t, J=7.7 Hz, 3H), 0.90 (t, J=7.0 Hz, 3H); ¹³C NMR (176 MHz, CDCl₃) δ 169.7, 153.1, 141.5, 138.9, 136.1, 131.2, 130.1, 127.8, 127.0, 117.9, 115.7, 61.0, 26.1, 15.4, 13.7, 12.5; HRMS (EI+) calcd for C₁₈H₂₀O₃ [M+]: 284.1413, found 284.1399.

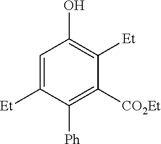 [SMILES: CCOC(=O)c1c(CC)c(O)cc(CC)c1-c1ccccc1]

### Ethyl 3,6-diethyl-4-hydroxy-[1,1′-biphenyl]-2-carboxylate (76)

5-Ethyl-3-hydroxy-6-phenyl-2H-pyran-2-one (43.2 mg, 0.2 mmol, 1 eq) and ethyl 3-nitropent-2-enoate (69.2 mg, 0.4 mmol, 2 eq) were subjected to the general procedure for 3 hours. Purification by FCC (15:1 hexanes:EtOAc) yielded 76 as a yellow solid (26.3 mg, 44%).

Data for 76: R_{f}: 0.33 (5:1 hexanes:EtOAc); mp: 94-97° C.; IR (thin film) 3399, 2969, 2935, 2873, 1700, 1598, 1443, 1309 cm⁻¹; ¹H NMR (700 MHz, CDCl₃) δ 7.33 (t, J=7.0 Hz, 2H), 7.31-7.30 (m, 1H), 7.23-7.21 (m, 2H), 6.75 (s, 1H), 5.08 (s, 1H), 3.92 (q, J=7.0 Hz, 2H), 2.61 (q, J=7.7 Hz, 2H), 2.35 (q, J=7.7 Hz, 2H), 1.22 (t, J=7.7 Hz, 3H), 1.00 (t, J=7.0 Hz, 3H), 0.91 (t, J=7.7 Hz, 3H); ¹³C NMR (176 MHz, CDCl₃) δ 169.6, 152.8, 141.5, 138.9, 135.9, 131.2, 130.1, 127.7, 127.0, 124.2, 116.0, 60.8, 26.1, 21.1, 15.3, 14.5, 13.7; HRMS (EI+) calcd for C₁₉H₂₂O₃ [M+]: 298.1569, found 298.1583.

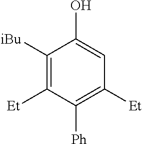

### 2,6-Diethyl-3-isobutyl-[1,1′-biphenyl]-4-ol (77)

5-Ethyl-3-hydroxy-4-isobutyl-6-phenyl-2H-pyran-2-one (27.2 mg, 0.1 mmol, 1 eq) and 1-nitrobut-1-ene (30.3 mg, 0.3 mmol, 3 eq) were subjected to the general procedure at 130° C. for 24 hours. Purification by FCC (50:1 hexanes:EtOAc) yielded 77 as a yellow solid (13.3 mg, 47%).

Data for 77: R_{f}: 0.48 (5:1 hexanes:EtOAc); mp: 55-58° C.; IR (thin film) 3424, 2960, 2931, 2869, 1600, 1463 cm⁻¹; ¹H NMR (700 MHz, CDCl₃) δ 7.39 (t, J=7.7 Hz, 2H), 7.33 (t, J=7.7 Hz, 1H), 7.17 (dd, J=7.7, 1.4 Hz, 2H), 6.59 (s, 1H), 4.61 (s, 1H), 2.56 (d, J=7.0 Hz, 2H), 2.37 (q, J=7.7 Hz, 2H), 2.20 (q, J=7.7 Hz, 2H), 1.97 (sept, J=7.0 Hz, 1H), 1.01-0.99 (m, 9H), 0.87 (t, J=7.7 Hz, 3H); ¹³C NMR (176 MHz, CDCl₃) δ 153.2, 142.4, 141.2, 141.1, 134.3, 130.3, 127.9, 126.4, 122.9, 112.4, 34.9, 29.5, 26.7, 23.6, 22.8, 15.3, 15.2; HRMS (EI+) calcd for C₂₀H₂₆O [M+]: 282.1984, found 282.1990.

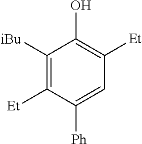

### 2,5-Diethyl-3-isobutyl-[1,1′-biphenyl]-4-ol (78)

5-Ethyl-3-hydroxy-4-isobutyl-6-phenyl-2H-pyran-2-one (27.2 mg, 0.1 mmol, 1 eq) and 2-nitrobut-1-ene (30.3 mg, 0.3 mmol, 3 eq) were subjected to the general procedure at 130° C. Purification by FCC (50:1 hexanes:EtOAc) yielded 78 as a yellow oil (18.4 mg, 65%).

Data for 78: R_{f}: 0.72 (5:1 hexanes:EtOAc); IR (thin film) 3594, 2961, 2932, 2869, 1458, 1196 cm⁻¹; ¹H NMR (700 MHz, CDCl₃) δ 7.38 (t, J=7.0 Hz, 2H), 7.32 (tt, J=7.0, 1.4 Hz, 1H), 7.30 (dd, J=7.0, 1.4 Hz, 2H), 6.84 (s, 1H), 4.67 (s, 1H), 2.64-2.56 (m, 6H), 1.97 (sept, J=7.0 Hz, 1H), 1.26 (t, J=7.7 Hz, 3H), 1.02 (d, J=7.0 Hz, 6H), 0.91 (t, J=7.7 Hz, 3H); ¹³C NMR (176 MHz, CDCl₃) δ 151.2, 143.2, 139.3, 134.8, 129.7, 128.2, 127.8, 126.3, 125.8, 125.2, 35.3, 29.5, 22.9, 22.9, 15.5, 13.7; HRMS (EI+) calcd for C₂₀H₂₆O [M+]: 282.1984, found 282.1974.

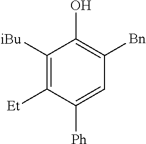

### 5-Benzyl-2-ethyl-3-isobutyl-[1,1′-biphenyl]-4-ol (79)

5-Ethyl-3-hydroxy-4-isobutyl-6-phenyl-2H-pyran-2-one (27.2 mg, 0.1 mmol, 1 eq) and (3-nitroallyl)benzene (21.9 mg, 0.3 mmol, 3 eq) were subjected to the general procedure. Purification by FCC (50:1 hexanes:EtOAc) yielded 79 as a yellow oil (21.9 mg, 64%).

Data for 79: R_{f}: 0.75 (6:1 hexanes:EtOAc); IR (thin film) 3548, 2957, 2929, 2868, 1602, 1461 cm⁻¹; ¹H NMR (700 MHz, CDCl₃) δ 7.28 (t, J=7.0 Hz, 2H), 7.33-7.30 (m, 5H), 7.26 (d, J=7.0 Hz, 2H), 7.22 (t, J=7.0 Hz, 1H), 6.86 (s, 1H), 4.62 (s, 1H), 3.99 (s, 2H), 2.61-2.57 (m, 4H), 1.95 (sept, J=7.0 Hz, 1H), 0.98 (d, J=7.0 Hz, 6H), 0.92 (t, J=7.0 Hz, 3H); ¹³C NMR (176 MHz, CDCl₃) δ 151.7, 143.0, 140.2, 139.7, 135.0, 130.2, 129.7, 128.8, 128.7, 127.8, 126.5, 126.3, 126.1, 123.3, 37.0, 35.2, 29.5, 22.9, 22.8, 15.5; HRMS (EI+) calcd for C₂₅H₂₈O [M+]: 344.2140, found 344.2149.

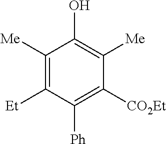 [SMILES: CCOC(=O)c1c(C)c(O)c(C)c(CC)c1-c1ccccc1]

### Ethyl 6-ethyl-4-hydroxy-3,5-dimethyl-[1,1′-biphenyl]-2-carboxylate (80)

5-Ethyl-3-hydroxy-4-methyl-6-phenyl-2H-pyran-2-one (46 mg, 0.2 mmol, 2 eq) and ethyl 3-nitrobut-2-enoate (15.9 mg, 0.1 mmol, 1 eq) were subjected to the general procedure for 3 days. Purification by FCC (10:1 hexanes:EtOAc) yielded 80 as a yellow oil (15.8 mg, 53%).

Data for 80: R_{f}: 0.36 (7:1 hexanes:EtOAc); IR (thin film) 3455, 2971, 2932, 1703, 1443, 1289, 1202 cm⁻¹; ¹H NMR (700 MHz, CDCl₃) δ 7.33 (tt, J=6.3, 1.4 Hz, 2H), 7.30 (tt, J=6.3, 1.4 Hz, 1H), 7.21 (dt, J=6.3, 1.4 Hz, 2H), 4.81 (s, 1H), 3.88 (q, J=7.0 Hz, 2H), 2.43 (q, J=7.7 Hz, 2H), 2.27 (s, 3H), 2.21 (s, 3H), 0.93 (t, J=7.7 Hz, 3H), 0.89 (t, J=7.0 Hz, 3H); ¹³C NMR (176 MHz, CDCl₃) δ 169.5, 151.5, 139.8, 139.8, 133.6, 131.4, 130.2, 127.6, 126.9, 122.7, 116.8, 60.7, 23.6, 14.6, 13.7, 13.0, 12.0; HRMS (EI+) calcd for C₁₉H₂₂O₃ [M+]: 298.1569, found 298.1574.

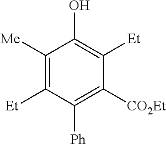 [SMILES: CCOC(=O)c1c(CC)c(O)c(C)c(CC)c1-c1ccccc1]

### Ethyl 3,6-diethyl-4-hydroxy-5-methyl-[1,1′-biphenyl]-2-carboxylate (81)

5-Ethyl-3-hydroxy-4-methyl-6-phenyl-2H-pyran-2-one (23 mg, 0.1 mmol, 1 eq) and ethyl 3-nitropent-2-enoate (86.5 mg, 0.5 mmol, 5 eq) were subjected to the general procedure for 2 days. Purification by FCC (20:1 hexanes:EtOAc) yielded 81 as a yellow oil (20.6 mg, 66%).

Data for 81: R_{f}: 0.45 (5:1 hexanes:EtOAc); IR (thin film) 3480, 2970, 2934, 1705, 1285, 1202 cm⁻¹; ¹H NMR (700 MHz, CDCl₃) δ 7.35 (t, J=7.0 Hz, 2H), 7.30 (t, J=7.0 Hz, 1H), 7.24 (d, J=7.0 Hz, 2H), 5.07 (s, 1H), 3.89 (q, J=7.0 Hz, 2H), 2.61 (q, J=7.0 Hz, 2H), 2.43 (q, J=7.0 Hz, 2H), 2.28 (s, 3H), 1.23 (t, J=7.0 Hz, 3H), 0.95 (t, J=7.0 Hz, 3H), 0.91 (t, J=7.0 Hz, 3H); ¹³C NMR (176 MHz, CDCl₃) δ 169.7, 151.2, 139.8, 133.2, 131.4, 130.3, 127.6, 126.9, 123.3, 123.0, 60.7, 23.7, 21.6, 14.6, 14.5, 13.7, 12.0; HRMS (ES+) calcd for C₂₀H₂₅O₃ [M+H]: 313.1804, found 313.1802.

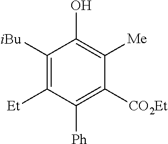

### Ethyl 6-ethyl-4-hydroxy-5-isobutyl-3-methyl-[1,1′-biphenyl]-2-carboxylate (82)

5-Ethyl-3-hydroxy-4-isobutyl-6-phenyl-2H-pyran-2-one (54.4 mg, 0.2 mmol, 1 eq) and ethyl 3-nitrobut-2-enoate (63.4 mg, 0.4 mmol, 2 eq) were subjected to the general procedure for 4 days. Purification by FCC (7:1 hexanes:EtOAc) yielded 82 as a yellow oil (48.1 mg, 71%).

Data for 82: R_{f}: 0.27 (7:1 hexanes:EtOAc); IR (thin film) 3478, 2958, 2932, 2869, 1706, 1430, 1296, 1202 cm⁻¹; ¹H NMR (700 MHz, CDCl₃) δ 7.33 (t, J=7.0 Hz, 2H), 7.30 (t, J=7.0 Hz, 1H), 7.23 (d, J=7.0 Hz, 2H), 4.82 (s, 1H), 3.89 (q, J=7.0 Hz, 2H), 2.58 (d, J=7.7 Hz, 2H), 2.45 (q, J=7.7 Hz, 2H), 2.20 (s, 3H), 1.95 (sept, J=7.0 Hz, 1H), 0.99 (d, J=7.0 Hz, 6H), 0.89 (t, J=7.0 Hz, 3H), 0.86 (t, J=7.7 Hz, 3H); ¹³C NMR (176 MHz, CDCl₃) δ 167.7, 151.6, 140.2, 139.8, 133.9, 131.5, 130.3, 127.6, 126.9, 126.6, 116.9, 60.8, 35.3, 29.4, 23.1, 22.8, 15.3, 13.7, 13.0; HRMS (ES+) calcd for C₂₂H₂₉O₃ [M+H]: 341.2117, found 341.2127.

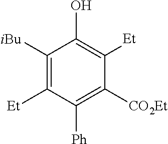

### Ethyl 3,6-diethyl-4-hydroxy-5-isobutyl-[1,1′-biphenyl]-2-carboxylate (83)

5-Ethyl-3-hydroxy-4-isobutyl-6-phenyl-2H-pyran-2-one (27.2 mg, 0.1 mmol, 1 eq) and ethyl 3-nitropent-2-enoate (34.6 mg, 0.2 mmol, 2 eq) were subjected to the general procedure at 180° C. for 3 days. Purification by FCC (15:1 hexanes:EtOAc) yielded 83 as a yellow oil (11.3 mg, 32%).

Data for 83: R_{f}: 0.45 (5:1 hexanes:EtOAc); IR (thin film) 3494, 2962, 2871, 1707, 1465 cm⁻¹; ¹H NMR (700 MHz, CDCl₃) δ 7.33 (t, J=7.0 Hz, 2H), 7.29 (t, J=7.0 Hz, 1H), 7.24 (d, J=7.0 Hz, 2H), 4.79 (s, 1H), 3.87 (q, J=7.0 Hz, 2H), 2.61-2.57 (m, 4H), 2.45 (q, J=7.7 Hz, 2H), 1.95 (sept, J=7.0 Hz, 1H), 1.22 (t, J=7.7 Hz, 3H), 1.00 (d, J=7.0 Hz, 6H), 0.90 (t, J=7.0 Hz, 3H), 0.86 (t, J=7.7 Hz, 3H); ¹³C NMR (176 MHz, CDCl₃) δ 169.6, 151.2, 140.2, 139.8, 133.6, 131.5, 130.4, 127.5, 126.8, 126.6, 123.1, 60.6, 35.4, 29.5, 23.1, 22.8, 21.6, 15.2, 14.4, 13.7; HRMS (EI+) calcd for C₂₃H₃₀O₃ [M+]: 354.2195, found 354.2180.

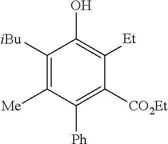

### Ethyl 3-ethyl-4-hydroxy-5-isobutyl-6-methyl-[1,1′-biphenyl]-2-carboxylate (84)

3-Hydroxy-4-isobutyl-5-methyl-6-phenyl-2H-pyran-2-one (258 mg, 0.1 mmol, 1 eq) and ethyl 3-nitropent-2-enoate (346 mg, 0.2 mmol, 2 eq) were subjected to the general procedure at 180° C. Purification by FCC (20:1 hexanes:EtOAc) yielded 84 as a yellow oil (249.3 mg, 70%).

Data for 84: R_{f}: 0.48 (5:1 hexanes:EtOAc); mp: 84-89° C.; IR (thin film) 3494, 2956, 2926, 2869, 1707, 1464, 1295, 1198 cm⁻¹; ¹H NMR (700 MHz, CDCl₃) δ 7.35 (t, J=7.0 Hz, 2H), 7.30 (t, J=7.0 Hz, 1H), 7.22 (d, J=7.0 Hz, 2H), 5.03 (s, 1H), 3.92 (q, J=7.0 Hz, 2H), 2.63 (q, J=7.0 Hz, 2H), 2.60 (d, J=7.7 Hz, 2H), 2.03 (s, 3H), 1.94 (sept, J=7.0 Hz, 1H), 1.24 (t, J=7.0 Hz, 3H), 1.01 (d, J=7.0 Hz, 6H), 0.91 (t, J=7.0 Hz, 3H); ¹³C NMR (176 MHz, CDCl₃) δ 169.8, 151.0, 140.4, 133.9, 133.2, 131.9, 130.1, 127.8, 127.6, 126.8, 123.4, 60.7, 36.0, 28.9, 22.8, 21.6, 17.4, 14.5, 13.7; HRMS (ES+) calcd for C₂₂H₂₉O₃ [M+H]: 341.2117, found 341.2121.

### Example 4

### Syntheses of Substituted Benzenes

### General Procedure for Stille Couplings

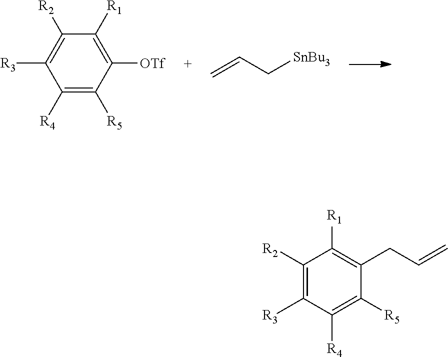

To a thick-walled reaction vessel were added the triflate (1 eq), Pd(PPh₃)₄ (10 mol %), and DMF (0.1 M) under Ar. The stannane (1.3 eq) was added dropwise, and the tube was sealed and heated to 90° C. for 16 hours unless otherwise noted. The reaction mixture was cooled to room temperature, EtOAc was added and the organic layer was washed with brine. The organic layer was dried over Na₂SO₄, filtered and concentrated. The residue was purified by FCC.

### General Procedure for Suzuki Couplings

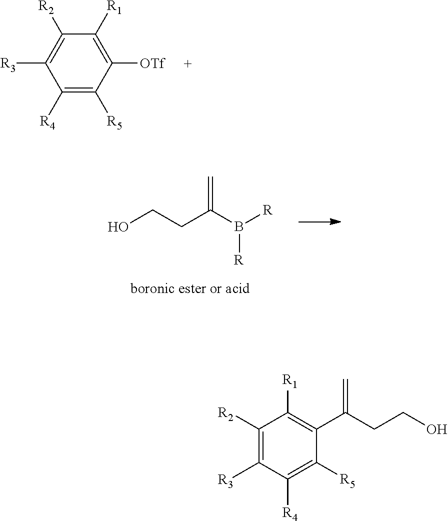

To a thick-walled reaction vessel were added the triflate (1 eq), boronic ester or acid (1.5 eq), Pd(PPh₃)₄ (10 mol %), K₂CO₃ (3 eq) and 1, 4-dioxane/water mixture (4:1, 0.1 M). The headspace of the vessel was evacuated by brief exposure to vacuum, and the vessel was back-filled with Ar. The vessel was evacuated and back-filled with Ar two additional times. The tube was sealed and heated to 110° C. for 16 hours unless otherwise noted. The reaction was cooled to room temperature, and solvent was removed under vacuum. The crude reaction mixture was dissolved in EtOAc, filtered through celite, dried over Na₂SO₄, filtered, and concentrated. The residue was purified by FCC.

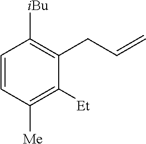

### 2-Allyl-3-ethyl-1-isobutyl-4-methylbenzene (85)

2-Ethyl-6-isobutyl-3-methylphenyl trifluoromethanesulfonate (21 mg, 0.07 mmol, 1 eq) and allyl tributylstannane (28 mg, 0.085 mmol, 1.2 eq) were subjected to the general Stille coupling procedure with a reaction time of 6 days. Purification by FCC (100% hexanes) yielded 85 as a colorless oil (8.8 mg, 63%).

Data for 85: R_{f}: 0.59 (Pure hexanes); IR (thin film) 3079, 2956, 2928, 2868, 1636, 1461 cm⁻¹; ¹H NMR (700 MHz, CDCl₃) δ 6.96 (d, J=7.7 Hz, 1H), 6.89 (d, J=7.7 Hz, 1H), 5.95 (ddt, J=16.8, 10.5, 5.6 Hz, 1H), 4.99 (dd, J=9.8, 1.4 Hz, 1H), 4.82 (dd, J=16.8, 1.4 Hz, 1H), 3.43 (dt, J=5.6, 2.1 Hz, 2H), 2.64 (q, J=7.7 Hz, 2H), 2.43 (d, J=7.7 Hz, 2H), 2.31 (s, 3H), 1.85 (sept, J=7.0 Hz, 1H), 1.12 (t, J=7.7 Hz, 3H), 1.92 (d, J=7.0 Hz, 6H); ¹³C NMR (176 MHz, CDCl₃) δ 141.3, 138.1, 137.6, 135.1, 133.6, 128.1, 127.8, 114.9, 42.6, 32.7, 29.5, 22.7, 22.7, 19.7, 14.3; HRMS (EI+) calcd for C₁₆H₂₄ [M+]: 216.1878, found 216.1884.

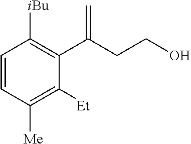

### 3-(2-Ethyl-6-isobutyl-3-methylphenyl)but-3-en-1-ol (86)

2-Ethyl-6-isobutyl-3-methylphenyl trifluoromethanesulfonate (21 mg, 0.07 mmol, 1 eq) and 3-(4,4,5,5-tetramethyl-1,3,2-dioxaborolan-2-yl)but-3-en-1-ol (18.3 mg, 0.09 mmol, 1.3 eq) were subjected to the general Suzuki coupling procedure with a reaction time of 4 days. Purification by FCC (5:1 hexanes:EtOAc) yielded 86 as a colorless oil (8.6 mg, 54%).

Data for 86: R_{f}: 0.38 (5:1 hexanes:EtOAc); IR (thin film) 3337, 2955, 2932, 2869, 1637, 1466 cm⁻¹; ¹H NMR (700 MHz, CDCl₃) δ 7.02 (d, J=7.7 Hz, 1H), 6.95 (d, J=7.7 Hz, 1H), 5.35 (dd, J=1.4 Hz, 1H), 4.94 (d, J=1.4 Hz, 1H), 3.86 (app q, J=6.3 Hz, 2H), 2.68-2.62 (m, 1H), 2.52-2.49 (m, 3H), 2.43 (dd, J=14.0, 7.0 Hz, 1H), 2.33 (dd, J=14.0, 7.0 Hz, 1H), 2.32 (s, 3H), 1.88 (sept, J=7.0 Hz, 1H), 1.41 (s, 1H), 1.10 (t, J=7.0 Hz, 3H), 0.92 (d, J=7.0 Hz, 3H), 0.87 (d, J=7.0 Hz, 3H); ¹³C NMR (176 MHz, CDCl₃) δ 145.1, 142.0, 139.4, 136.2, 133.5, 128.8, 126.8, 115.0, 60.5, 42.1, 41.3, 29.8, 23.8, 22.7, 22.6, 19.4, 15.1; HRMS (ES+) calcd for C₁₇H₂₆ONa [M+Na]: 269.1881, found 269.1875.

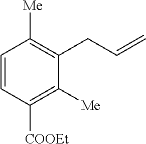 [SMILES: C=CCc1c(C)ccc(C(=O)OCC)c1C]

### Ethyl 3-allyl-2,4-dimethylbenzoate (87)

Ethyl 2,4-dimethyl-3-(((trifluoromethyl)sulfonyl)oxy)benzoate (32.6 mg, 0.1 mmol, 1 eq) and allyl tributylstannane (40 mg, 0.12 mmol, 1.2 eq) were subjected to the general Stille coupling procedure. Purification by FCC (50:1 hexanes:EtOAc) yielded 87 as a yellow oil (19.2 mg, 88%).

Data for 87: R_{f}: 0.74 (10:1 hexanes:EtOAc); IR (thin film) 3082, 2979, 2933, 1717, 1264 cm⁻¹; ¹H NMR (700 MHz, CDCl₃) δ 7.57 (d, J=7.7 Hz, 1H), 7.05 (d, J=7.7 Hz, 1H), 5.91 (ddt, J=16.8, 9.8, 4.9 Hz, 1H), 5.01 (dq, J=10.5, 2.1 Hz, 1H), 4.82 (dq, J=17.5, 2.1 Hz, 1H), 4.35 (q, J=7.0 Hz, 2H), 3.45 (dt, J=5.6, 2.1 Hz, 2H), 2.48 (s, 3H), 2.33 (s, 3H), 1.38 (t, J=7.7 Hz, 3H); ¹³C NMR (176 MHz, CDCl₃) δ 168.9, 140.8, 137.6, 137.4, 134.7, 129.7, 127.8, 127.5, 115.2, 60.7, 33.5, 27.9, 26.9, 20.5, 17.5, 16.5, 14.4, 13.6; HRMS (ES+) calcd for C₁₄H₁₉O₂ [M+H]: 219.1385, found 219.1384.

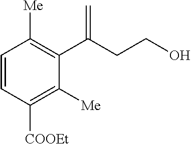 [SMILES: C=C(CCO)c1c(C)ccc(C(=O)OCC)c1C]

### Ethyl 3-(4-hydroxybut-1-en-2-yl)-2,4-dimethylbenzoate (88)

Ethyl 2,4-dimethyl-3-(((trifluoromethyl)sulfonyl)oxy)benzoate (32.6 mg, 0.1 mmol, 1 eq) and 3-(4,4,5,5-tetramethyl-1,3,2-dioxaborolan-2-yl)but-3-en-1-ol (26 mg, 0.13 mmol, 1.3 eq) were subjected to the general Suzuki coupling procedure. Purification by FCC (5:1 hexanes:EtOAc) yielded 88 as a yellow oil (20 mg, 81%).

Data for 88: R_{f}: 0.34 (3:1 hexanes:EtOAc); IR (thin film) 3419, 2980, 2931, 1716, 1591 cm⁻¹; ¹H NMR (700 MHz, CDCl₃) δ 7.64 (d, J=7.7 Hz, 1H), 7.08 (d, J=7.7 Hz, 1H), 5.41 (q, J=1.4 Hz, 1H), 4.94 (d, J=1.4 Hz, 1H), 4.34 (q, J=7.0 Hz, 2H), 3.82-3.76 (m, 2H), 2.51-2.49 (m, 2H), 2.47 (s, 3H), 2.29 (s, 1H), 1.57 (brs, 1H), 1.38 (t, J=7.0 Hz, 3H); ¹³C NMR (176 MHz, CDCl₃) δ 168.4, 144.9, 143.5, 139.3, 136.3, 128.8, 128.6, 127.3, 116.0, 60.7, 60.4, 40.3, 20.7, 17.9, 14.4; HRMS (ES+) calcd for C₁₅H₂₁O₃ [M+H]: 249.1491, found 249.1497.

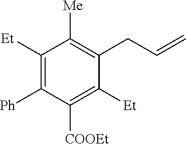 [SMILES: C=CCc1c(C)c(CC)c(-c2ccccc2)c(C(=O)OCC)c1CC]

### Ethyl 4-allyl-3,6-diethyl-5-methyl-[1,1′-biphenyl]-2-carboxylate (89)

Ethyl 3,6-diethyl-5-methyl-4-(((trifluoromethyl)sulfonyl)oxy)-[1,1′-biphenyl]-2-carboxylate (44.4 mg, 0.1 mmol, 1 eq) and allyl tributylstannane (43 mg, 0.13 mmol, 1.3 eq) were subjected to the general Stille couplings procedure. Purification by FCC (40:1 hexanes:Acetone) yielded 89 as a colorless oil (32 mg, 95%).

Data for 89: R_{f}: 0.57 (10:1 hexanes:Acetone); IR (thin film) 3082, 2974, 2934, 2875, 1726, 1274 cm⁻¹; ¹H NMR (700 MHz, CDCl₃) δ 7.34 (t, J=7.0 Hz, 2H), 7.30 (t, J=7.0 Hz, 1H), 7.25 (d, J=7.0 Hz, 2H), 5.97 (oct, J=5.1 Hz, 1H), 5.07 (d, J=10.5 Hz, 1H), 4.94 (d, J=17.5 Hz, 1H), 3.86 (q, J=7.0 Hz, 2H), 3.51 (d, J=3.5 Hz, 2H), 2.61 (q, J=7.0 Hz, 2H), 2.44 (q, J=7.0 Hz, 2H), 2.31 (s, 3H), 1.20 (t, J=7.0 Hz, 3H), 0.94 (t, J=7.0 Hz, 3H), 0.90 (t, J=7.0 Hz, 3H); ¹³C NMR (176 MHz, CDCl₃) δ 170.3, 139.9, 138.7, 137.1, 136.7, 136.1, 135.7, 135.6, 133.4, 129.9, 127.5, 126.9, 115.5, 60.5, 33.5, 24.4, 23.8, 15.8, 15.7, 14.6, 13.7; HRMS (ES+) calcd for C₂₃H₂₉O₂ [M+H]: 337.2168, found 337.2164.

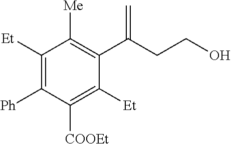 [SMILES: C=C(CCO)c1c(C)c(CC)c(-c2ccccc2)c(C(=O)OCC)c1CC]

### Ethyl 3,6-diethyl-4-(4-hydroxybut-1-en-2-yl)-5-methyl-[1,1′-biphenyl]-2-carboxylate (90)

Ethyl 3,6-diethyl-5-methyl-4-(((trifluoromethyl)sulfonyl)oxy)-[1,1′-biphenyl]-2-carboxylate (23 mg, 0.052 mmol, 1 eq) and (4-hydroxybut-1-en-2-yl)boronic acid (12 mg, 0.104 mmol, 2 eq) were subjected to the general Suzuki coupling procedure in the microwave with reaction time of 20 hours. Purification by FCC (5:1 hexanes:EtOAc) yielded 90 as a yellow oil (11.9 mg, 63%).

Data for 90: R_{f}: 0.12 (5:1 hexanes:EtOAc); IR (thin film) 3441, 2970, 2934, 2876, 1723 cm⁻¹; ¹H NMR (700 MHz, CDCl₃) δ 7.35 (t, J=7.0 Hz, 2H), 7.31 (tt, J=7.0, 1.4 Hz, 1H), 7.27-7.26 (m, 2H), 7.23 (dd, J=7.7, 2.1 Hz, 1H), 5.42 (d, J=1.4 Hz, 1H), 5.05 (d, J=1.4 Hz, 1H), 3.89-3.88 (m, 2H), 3.87-3.82 (m, 2H), 2.65-2.60 (m, 1H), 2.58-2.51 (m, 3H), 2.43 (q, J=7.7 Hz, 2H), 2.29 (s, 3H), 1.49 (brs, 1H), 1.13 (t, J=7.0 Hz, 3H), 0.93 (t, J=7.0 Hz, 3H), 0.89 (t, J=7.0 Hz, 3H); ¹³C NMR (176 MHz, CDCl₃) δ 170.1, 145.2, 142.0, 139.7, 138.7, 137.9, 134.6, 134.5, 133.0, 129.9, 127.6, 127.6, 127.0, 115.5, 60.5, 60.5, 40.8, 24.5, 23.7, 16.7, 16.6, 14.3, 13.7; HRMS (ES+) calcd for C₂₄H₃₁O₃ [M+H]: 367.2273, found 367.2267.

### Example 5

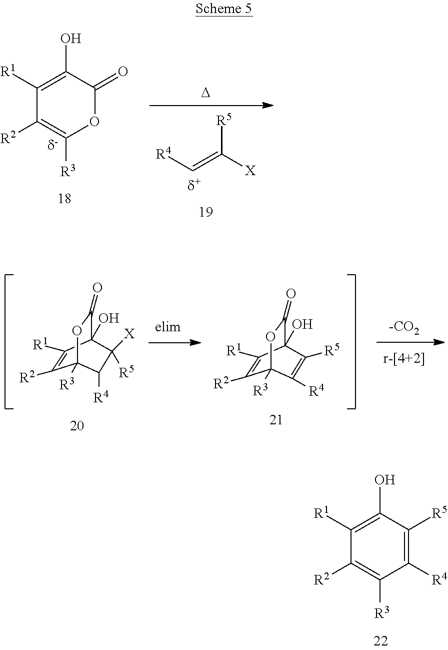

With respect to Scheme 5, R¹, R², R³, R⁴, R⁵ and X are as defined above for Scheme 4. It was hypothesized that the reactivity and regioselectivity limitations of the Diels-Alder-based strategies could be overcome if both coupling partners are electronically polarized. Specifically, starting materials 18 and 19 (Scheme 5) would show both enhanced reactivity and give programmable regioselectivity in the products. An alkyne-equivalent dienophile (19) was identified that facilitated the initial Diels-Alder cycloaddition at lower temperatures and provided regioselectivity in the cycloaddition giving 20 (Scheme 5). The activating group X would have to double as a leaving group, undergoing elimination to give bicycle 21, prior to the final retro-cycloaddition that liberates CO₂ and delivers the product 22.

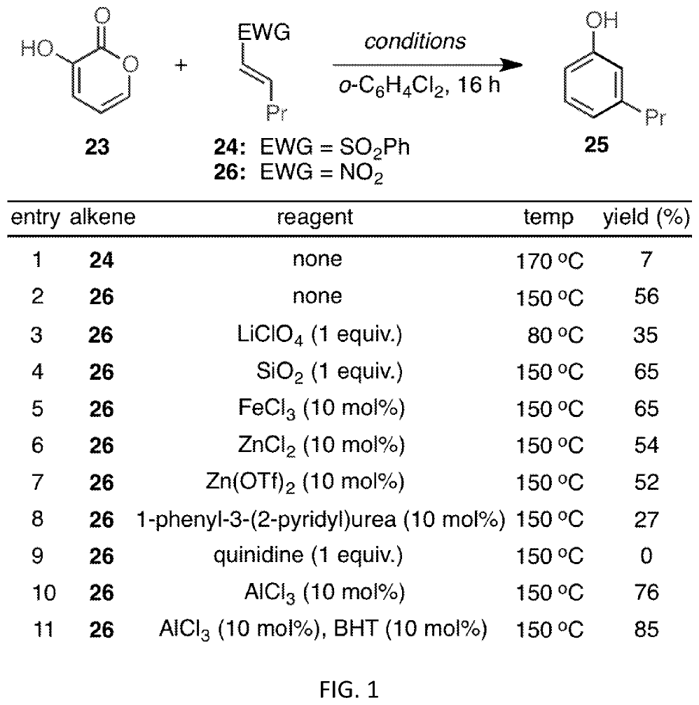

Initial experimentation focused on combining a hydroxyl-pyrone diene 23 with a vinyl sulfone dienophile (24, FIG. 1). Both coupling partners were suitably polarized such that single regioisomeric phenol 25 was formed. However, although initial experiments successfully produced the desired phenol compound, successful cycloadditions often required high temperatures, and substantial decomposition of the reactants was observed. The requirement of forcing conditions was attributed to sluggish reactivity of the vinyl sulfone, and a more reactive dienophile was sought.

Nitroalkenes are potent dienophiles, and the cycloaddition cascade with nitroalkene 26 (FIG. 1, entry 2) was investigated. Gratifyingly, the cycloaddition cascade was successful, and it occurred at relatively mild temperatures. Phenol 25 was observed as the major product, albeit in modest yield. To the best of the inventors' knowledge, this represents the first reaction of a pyrone and a nitroalkene to give a benzene product.

Addition of reagents such as Lewis acids facilitated the reaction proceeding at lower temperatures (FIG. 1, entries 3-8). However, little improvement in the reaction of 23 and 26 was observed in the presence of quinidine (FIG. 1, entry 9), a known catalyst of hydroxypyrone Diels-Alder reactions. It was discovered that AlCl₃ promoted the reaction such that it occurred on useful timescales and in high yield (FIG. 1, entry 10).

Addition of a radical initiator, such as butylated hydroxytoluene (BHT), also enhanced the reaction yield. Without being bound to a particular theory, a radical initiator likely prevents decomposition of the reagents at elevated temperatures (FIG. 1, entry 11). Additionally, the reaction delivered one observable phenol isomer by NMR, thereby demonstrating the regioselectivity of the reaction. Analysis by gas chromatography indicated that the regioisomer ratio (rr) was 33:1. Activation may involve deprotonation of the hydroxyl group, and the related dienes 3-methylpyrone, 3-methoxypyrone, and 3-dibutylaminopyrone did not react with 26 under identical conditions with AlCl₃.

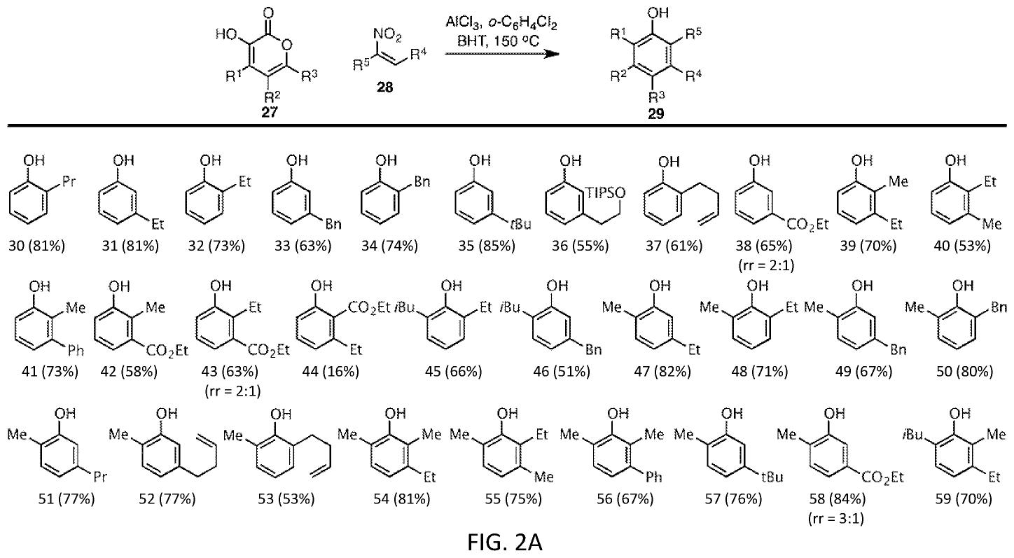

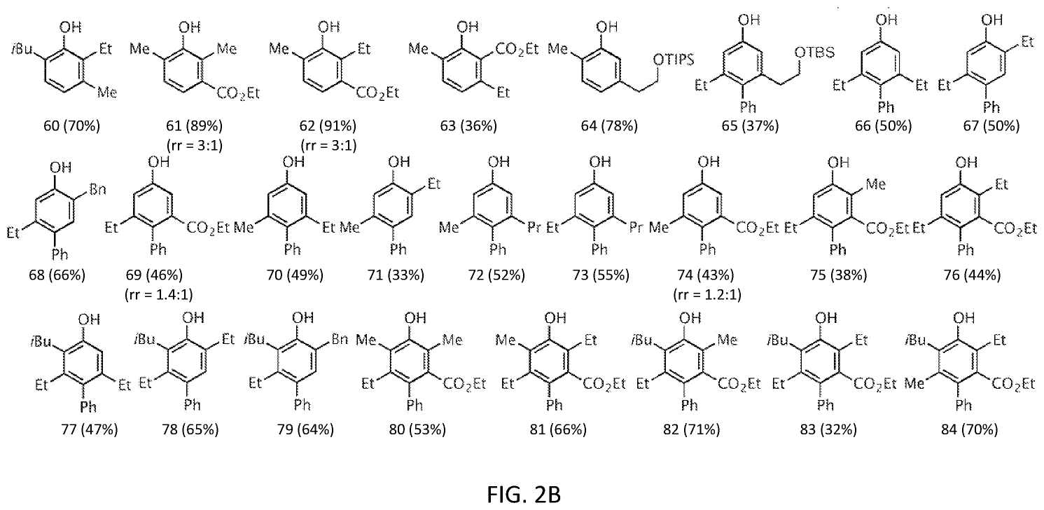

The regioisomeric nitroalkene (FIG. 2A, 28, R⁴=H, R⁵═Pr) was then subjected to the AlCl₃/BHT reaction conditions. The regioisomeric ortho-substituted phenol 30 was obtained as a single observable product (rr=125:1, GC). This result demonstrated that a regiospecific synthesis of phenols could be realized through a cascade Diels-Alder-elimination-retro-Diels-Alder process.

The phenol synthesis was further investigated using an expanded set of coupling partners. Other simple nitroalkenes bearing a single alkyl group readily participated in the reaction, and the same regiospecificity was observed. Isomeric nitroalkenes bearing alkyl groups gave regioisomeric pairs of products (FIG. 2A, 31 & 32; 33 & 34). Bulky substituents were tolerated on the nitroalkene (FIG. 2A, 35), as well as silyl ethers (FIG. 2A, 36), and unactivated alkenes (FIG. 2A, 37). When the nitroalkene dienophile contained an additional electron-withdrawing group that competed for polarization of the dieneophile, the nitro group still controlled the regioselectivity, but the reaction occasionally showed decreased regioselectivity (FIG. 2A, 38, rr=2:1).

Nitroalkenes with increased substitution (FIG. 2A, 28, R⁴ and R⁵≠H) were conveniently prepared using the Henry reaction. They also participated in the reaction to give 2,3-disubstituted phenols. Disubstituted phenol 39 was obtained as a single observable (NMR) isomer. As above, the reaction was regiospecific, and regioisomeric phenol 40 was produced selectively. Note that such 2,3-disubstituted phenols are not products obtained from substitution of either 2-alkylphenols or 3-alkylphenols. Phenol 39 was previously prepared in 6 steps featuring a ring-closing metathesis to construct the six-membered ring, and isomer 40 has not been prepared previously to the inventors' knowledge. Other nitroalkenes bearing two substituents participated in the phenol synthesis, and aromatic (FIG. 2A, 41) and ester (FIG. 2A, 42, 43 & 44) groups were well tolerated. As above, when the nitroalkene bore an ester group, the nitro functional group dictated the major regioisomer; however, when the ester competed for alkene polarization (FIG. 2A, 43), the regioisomer ratio was lower. When the ester and nitro groups were located on the same carbon, the regioselectivity was high (FIG. 2A, 44); however this reactant was very reactive and prone to decomposition.

Higher substitution on the pyrone was also well tolerated. 3-Hydroxypyrone could be regioselectively transformed to 4-alkyl-3-hydroxy-2-pyrones in two steps (FIG. 2A, 27, R¹=alkyl). These substrates also underwent the pericyclic cascade reaction to give phenols in good yields (FIGS. 2A and 2B, 45-64). As above, the reaction was regiospecific with respect to the nitroalkene dienophile and regioisomeric pairs could be prepared with nitroalkenes bearing one alkyl group (FIG. 2A, 47 & 48; 49 & 50; 52 & 53), or with two alkyl groups (FIGS. 2A and 2B, 54 & 55; 59 & 60; 62 & 63). The reaction successfully prepared a variety of other 2,5-disubstituted, 2,6-disubstituted, and 2,5,6-trisubstituted phenols with control of substituent radiochemistry.

Matsuda's one-step Rh-catalyzed synthesis of pyrones was used to prepare 5,6-disubstituted 3-hydroxypyrones (FIG. 2A, 27, R² and R³≠H). These pyrones reacted to give 3,4,5-trisubstituted or 3,4,6-trisubstituted phenols (FIG. 2B, 65-74). As before, the reaction gives pairs of phenols with regiospecificity (FIG. 2B, 66 & 67; 70 & 71) based on the choice of nitroalkene starting material. Reaction with more substituted nitroalkenes gave tetrasubstituted phenols 75 and 76. Finally, fully substituted pyrones were prepared (FIG. 2, 27, R¹, R², and R³≠H), and they reacted with nitroalkenes to give tetrasubstituted phenols (FIG. 2B, 77-79) and penta-substituted (i.e., fully-substituted) phenols (FIG. 2B, 8044) with complete control of radiochemistry. This method represents a direct synthesis of substituted benzene rings with up to six different substituents with complete control of substitution pattern. As such, this represents a major advance in the preparation of highly substituted benzenoid molecules.

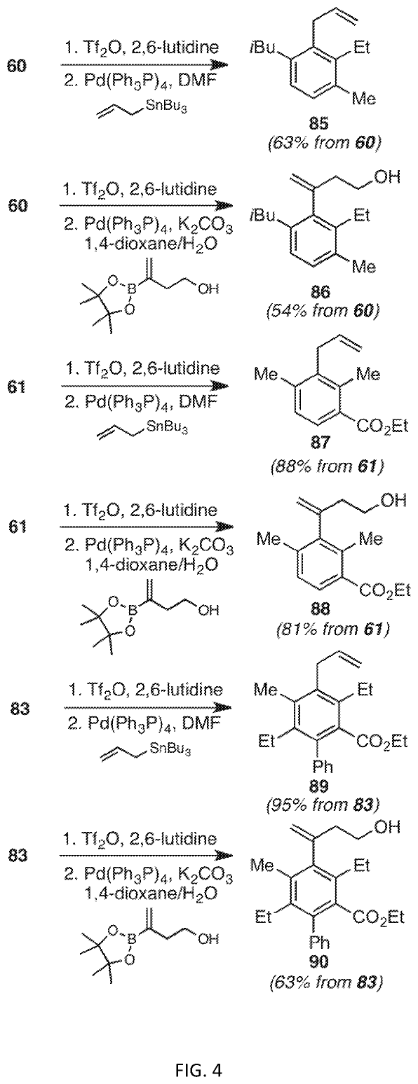

A major reason for the interest in phenols is their ability to participate in bond formations using the hydroxy functional group. This allows for the conversion of phenols into substituted benzenes of interest (FIG. 4). The ability of the disclosed highly substituted phenols to couple with organometallic reagents to give highly substituted benzenes was evaluated. Phenol 60 underwent Stille cross-coupling to give allyl benzene 85 (FIG. 4), and it participated in a Suzuki-Miyaura coupling to give 86 (FIG. 4). The ester-containing phenol 61 also reacted under the same conditions to give high yields of benzenes 87, and 88, respectively (FIG. 4). Finally, fully substituted phenol 83 was converted by Stille and Suzuki couplings to give 89 and 90, respectively (FIG. 4).

In conclusion, disclosed herein a direct synthesis of substituted phenols from 3-hydroxypyrones and nitroalkenes that is regiospecific with respect to the alkene. Moreover, high levels of substitution are tolerated and fully substituted phenols are prepared by the method. Yields are synthetically useful and compare favorably with classic and modern phenol syntheses. The programmable nature of the reaction allows non-canonical substitution patterns to be constructed. Finally, conversion of the phenols to the corresponding benzenes allows synthesis of highly substituted benzenes with full control of substituent location.

### Example 6

All reactions were carried out under an inert Argon atmosphere in oven-dried glassware. External (heated oil or cryogenic solvent) bath temperatures were used to record all reaction temperatures. Flash column chromatography (FCC) was carried out with SiliaFlash P60, 60 Å silica gel. Reactions and column chromatography were monitored with EMD silica gel 60 F254 plates and visualized with potassium permanganate stain. Reagent grade 1,4-dioxane was dried over CaH₂ and distilled prior to use. 1,2-Dichlorobenzene (DCB) was distilled under reduced pressure and degassed using three freeze-pump-thaw cycles. Tetrahydrofuran (THF), toluene, and benzene were dried by passage through activated alumina columns. All other reagents and solvents were used without further purification from commercial sources. Unless otherwise noted, melting points were obtained from material that solidified after chromatography.

Instrumentation: FT-IR spectra were obtained on NaCl plates with a PerkinElmer Spectrum Vision spectrometer. HRMS were recorded on a JEOL MS Route Magnetic Sector Instrument (EI) or a Waters Synapt HDMS TOF instrument (ESI). Proton and carbon NMR spectra (¹H NMR and ¹³C NMR) were recorded in deuterated chloroform (CDCl₃) unless otherwise noted on a Bruker 700 MHz Avance III Spectrometer with carbon-optimized cryoprobe or Bruker 400 MHz DPX-400 spectrometer. Multiplicities are abbreviated as follows: s=singlet, d=doublet, t=triplet, q=quartet, sept=septet, m=multiplet. Melting points were determined with a Cole-Parmer instrument and are uncorrected.

### General Procedure for Benzofuranone Syntheses:

To a thick-walled reaction vessel were added the pyrone (2 equiv), nitroalkene (1 equiv), butylated hydroxytoluene (BHT) (0.1 equiv) and AlCl₃ (0.1 equiv). The vessel was flushed with Ar gas for 5 minutes. 1,2-Dichlorobenzene (DCB) (0.5 M) and trifluoroacetic acid (TFA) (0.2 equiv) were added, and tube was quickly sealed. The reaction mixture was heated to 120° C. for 16 hours unless otherwise noted. The reaction mixture was cooled to room temperature, and the mixture was directly purified by flash column chromatography (FCC) without aqueous work up.

Benzofuran-2(3H)-one (105): 3-Hydroxy-2H-pyran-2-one (44.8 mg, 0.4 mmol) and methyl 3-nitrobut-3-enoate (29 mg, 0.2 mmol) were subjected to the general procedure for 20 hours. Purification by FCC (20:1 hexanes:EtOAc) yielded 105 as a solid (17.1 mg, 64%). Spectroscopic data for 105 matched literature values.

7-Methylbenzofuran-2(3H)-one (106): 3-Hydroxy-4-methyl-2H-pyran-2-one (50.4 mg, 0.4 mmol) and methyl 3-nitrobut-3-enoate (29 mg, 0.2 mmol) were subjected to the general procedure for 4 hours. Purification by FCC (20:1 hexanes:EtOAc) yielded 106 as a solid (22.4 mg, 76%). Spectroscopic data for 106 matched literature values.

7-Isobutylbenzofuran-2(3H)-one (107): 3-Hydroxy-4-isobutyl-2H-pyran-2-one (50.4 mg, 0.3 mmol) and methyl 3-nitrobut-3-enoate (21.75 mg, 0.15 mmol) were subjected to the general procedure for 6 hours. Purification by FCC (20:1 hexanes:EtOAc) yielded 107 as an oil (14.4 mg, 51%). Data for 107: Rr 0.51 (3:1 hexanes:EtOAc); IR (thin film) 2957, 1811, 1449 cm⁻¹; ¹H NMR (700 MHz, CDCl₃) δ 7.11 (d, J=7.7 Hz, 1H), 7.09 (d, J=7.7 Hz, 1H), 7.05 (t, J=7.7 Hz, 1H), 3.74 (s, 2H), 2.54 (d, J=7.0 Hz, 2H), 1.96 (sept, J=6.3 Hz, 1H), 0.92 (dd, J=6.3, 0.7 Hz, 6H); ¹³C NMR (176 MHz, CDCl₃) δ 174.5, 153.3, 130.3, 125.0, 123.7, 122.7, 122.0, 38.8, 33.5, 29.0, 22.3; HRMS (ESI) m/z: [M+H]⁺ Calcd for C₁₂H₁₅O₂ 191.1072; Found 191.1079.

7-Phenylbenzofuran-2(3H)-one (108): 3-Hydroxy-4-phenyl-2H-pyran-2-one (28 mg, 0.15 mmol, 1.5 equiv) and methyl 3-nitrobut-3-enoate (14.5 mg, 0.1 mmol, 1 equiv) were subjected to the general procedure at 100° C. for 4 hours. Purification by FCC (50:1 hexanes:EtOAc) yielded 108 as a solid (12.7 mg, 60%). Data for 108: Rr 0.5 (3:1 hexanes:EtOAc); mp: 124-126° C.; IR (thin film) 1807, 1425 cm⁻¹; ¹H NMR (400 MHz, CDCl₃) δ 7.67-7.66 (m, 2H), 7.48-7.44 (m, 3H), 7.40-7.36 (m, 1H), 7.27-7.20 (m, 2H), 3.81 (s, 2H); ¹³C NMR (176 MHz, CDCl₃) δ 174.0, 151.7, 135.3, 129.0, 128.7, 128.6, 128.0, 125.1, 124.5, 123.7, 123.5, 33.1; HRMS (ESI) m/z: [M+H]⁺ Calcd for C₁₄H₁₅O₂ 211.0759; Found 211.0763.

6-Methyl-5-phenylbenzofuran-2(3H)-one (109): 3-Hydroxy-5-methyl-6-phenyl-2H-pyran-2-one (20.2 mg, 0.1 mmol, 1 equiv) and methyl 3-nitrobut-3-enoate (29 mg, 0.2 mmol, 2 equiv) were subjected to the general procedure. Purification by FCC (40:1 hexanes:EtOAc) yielded 109 as a solid (7.5 mg, 33%). Data for 109: Rr 0.54 (3:1 hexanes:EtOAc); mp: 104-109° C.; IR (thin film) 3029, 2958, 2924, 1806, 1632, 1481, 1061 cm⁻¹; ¹H NMR (700 MHz, CDCl₃) δ 7.42 (t, J=7.7 Hz, 2H), 7.36 (t, J=7.7 Hz, 1H), 7.27 (d, J=7.7 Hz, 2H), 7.13 (s, 1H), 7.02 (s, 1H), 3.74 (s, 2H), 2.27 (s, 3H); ¹³C NMR (176 MHz, CDCl₃) δ 174.6, 154.0, 141.1, 138.1, 136.8, 129.3, 128.3, 127.1, 125.8, 120.3, 112.2, 32.9, 21.0; HRMS (ESI) m/z: [M+H]⁺ Calcd for C₁₅H₁₃O₂ 225.0916; Found 225.0897.

6-Ethyl-7-methyl-5-phenylbenzofuran-2(3H)-one (110): 5-Ethyl-3-hydroxy-4-methyl-6-phenyl-2H-pyran-2-one (34.5 mg, 0.15 mmol, 1 equiv) and methyl 3-nitrobut-3-enoate (55 mg, 0.375 mmol, 2.5 equiv) were subjected to the general procedure at 100° C. Purification by FCC (20:1 hexanes:EtOAc) yielded 110 as a solid (27.3 mg, 72%). Data for 110: Rr 0.60 (4:1 hexanes:EtOAc); mp: 82-85° C.; IR (thin film) 2968, 1807, 1627, 1447, 1082 cm⁻¹; ¹H NMR (400 MHz, CDCl₃) δ 7.44-7.34 (m, 3H), 7.26-7.24 (m, 2H), 6.95 (s, 1H), 3.75 (s, 2H), 2.57 (q, J=7.2 Hz, 2H), 2.35 (s, 3H), 0.99 (t, J=7.2 Hz, 3H); ¹³C NMR (176 MHz, CDCl₃) δ 174.7, 153.2, 142.0, 141.7, 138.2, 129.3, 128.1, 126.3, 123.4, 119.4, 119.3, 33.6, 23.3, 14.7, 11.9; HRMS (ESI) m/z: [M+H]⁺ Calcd for C₁₇H₁₇O₂ 253.1229; Found 253.1221.

6-Ethyl-7-isobutyl-5-phenylbenzofuran-2(3H)-one (111): 5-Ethyl-3-hydroxy-4-isobutyl-6-phenyl-2H-pyran-2-one (27 mg, 0.1 mmol, 1 equiv) and methyl 3-nitrobut-3-enoate (29 mg, 0.2 mmol, 2 equiv) were subjected to the general procedure for 3 hours. Purification by FCC (100% hexanes then 20:1 hexanes:EtOAc) yielded 111 as an oil (14.3 mg, 49%). Data for 111: Rr 0.63 (5:1 hexanes:EtOAc); IR (thin film) 2958, 1809, 1445, 1102 cm⁻¹; ¹H NMR (700 MHz, CDCl₃) δ 7.40 (t, J=7.0 Hz, 2H), 7.35 (t, J=7.0 Hz, 1H), 7.26 (d, J=7.0 Hz, 2H), 6.94 (s, 1H), 3.74 (s, 2H), 2.63 (d, J=7.7 Hz, 2H), 2.60 (q, J=7.0 Hz, 2H), 2.00 (sept, J=7.0 Hz, 1H), 0.98 (d, J=7.0 Hz, 6H), 0.90 (t, J=7.7 Hz, 3H); ¹³C NMR (176 MHz, CDCl₃) δ 174.8, 153.4, 142.2, 141.8, 138.5, 129.4, 128.0, 126.9, 123.8, 123.4, 119.4, 35.3, 33.5, 29.4, 22.7, 22.6, 15.4; HRMS (ESI) m/z: [M+H]⁺ Calcd for C₂₀H₂₃O₂ 295.1698; Found 295.1685.

7-Isobutyl-6-methyl-5-phenylbenzofuran-2(3H)-one (112): 3-Hydroxy-4-isobutyl-5-methyl-6-phenyl-2H-pyran-2-one (26 mg, 0.10 mmol, 1 equiv) and methyl 3-nitrobut-3-enoate (36 mg, 0.25 mmol, 2.5 equiv) were subjected to the general procedure at 100° C. for 6 hours. Purification by FCC (50:1 hexanes:EtOAc) yielded 112 as a solid (24.1 mg, 86%). Data for 112: Rr 0.51 (5:1 hexanes:EtOAc); mp: 130-132° C.; IR (thin film) 2957, 2868, 1809, 1452, 1102 cm⁻¹; H NMR (400 MHz, CDCl₃) δ 7.43-7.39 (m, 2H), 7.35 (tt, J=6.4, 1.6 Hz, 1H), 7.26-7.24 (m, 2H), 6.99 (s, 1H), 3.75 (s, 2H), 2.64 (d, J=7.2 Hz, 2H), 2.18 (s, 3H), 1.98 (sept, J=6.8 Hz, 1H), 0.98 (d, J=6.8 Hz, 6H); ¹³C NMR (176 MHz, CDCl₃) δ 174.8, 153.2, 142.2, 138.7, 135.2, 129.4, 128.2, 126.9, 124.1, 123.3, 119.5, 35.9, 33.6, 28.8, 22.6, 17.2; HRMS (ESI) m/z: [M+H]⁺ Calcd for C₁₉H₂₁O₂ 281.1542; Found 281.1531.

4,7-Dimethylbenzofuran-2(3H)-one (113): 3-Hydroxy-4-methyl-2H-pyran-2-one (50.4 mg, 0.4 mmol) and methyl-3-nitropent-3-enoate (31.8 mg, 0.2 mmol) were subjected to the general procedure at 150° C. for 3 days. Purification by FCC (20:1 hexanes:EtOAc) yielded 113 as a solid (19.4 mg, 60%). Spectroscopic data for 113 matched literature values.

7-Isobutyl-4-methylbenzofuran-2(3H)-one (114): 3-Hydroxy-4-isobutyl-2H-pyran-2-one (50 mg, 0.30 mmol) and methyl-3-nitropent-3-enoate (24 mg, 0.15 mmol) were subjected to the general procedure at 150° C. for 22 hours. Purification by FCC (20:1 hexanes:EtOAc) yielded 114 as an oil (12.3 mg, 40%). Data for 114: Rr 0.6 (4:1 hexanes:EtOAc); IR (thin film) 2956, 1807, 1419, 1124 cm⁻¹; ¹H NMR (700 MHz, CDCl₃) δ 6.99 (d, J=7.7 Hz, 1H), 6.87 (d, J=7.7 Hz, 1H), 3.64 (s, 2H), 2.50 (d, J=7.7, 2H), 2.25 (s, 3H), 1.93 (sept, J=7.0 Hz, 1H), 0.91 (d, J=7.0 Hz, 6H); ¹³C NMR (176 MHz, CDCl₃) δ 174.6, 152.9, 131.9, 130.1, 124.7, 122.0, 121.7, 38.6, 32.6, 29.0, 22.3, 18.5; HRMS (ESI) m/z: [M+H]⁺ Calcd for C₁₃H₁₇O₂ 205.1229; Found 205.1226.

Methyl 7-methyl-2-oxo-2,3-dihydrobenzofuran-4-carboxylate (115): 3-Hydroxy-4-methyl-2H-pyran-2-one (38 mg, 0.30 mmol) and dimethyl-3-nitropent-2-enedioate (30.5 mg, 0.15 mmol) were subjected to the general procedure at 150° C. for 2 hours. Purification by FCC (20:1 hexanes:EtOAc) yielded 115 as a solid (13.1 mg, 42%). Data for 115: Rr 0.43 (5:1 hexanes:EtOAc); mp: 123-124° C. IR (thin film) 2956, 1804, 1706, 1304 cm⁻¹; ¹H NMR (700 MHz, CDCl₃) δ 7.70 (d, J=7.7 Hz, 1H), 7.21 (d, J=7.7 Hz, 1H), 4.04 (s, 2H), 3.92 (s, 3H), 2.37 (s, 3H); ¹³C NMR (176 MHz, CDCl₃) δ 174.1, 165.9, 153.5, 130.2, 126.3, 125.2, 124.8, 124.2, 52.3, 35.0, 15.6; HRMS (ESI) m/z: [M+H]⁺ Calcd for C₁₁H₁₁O₄ 207.0657; Found 207.0667.

Methyl 2-(2-methoxy-2-oxoethyl)-7-methylbenzofuran-4-carboxylate (116): To a thick-walled reaction vessel was added methyl 7-methyl-2-oxo-2,3-dihydrobenzofuran-4-carboxylate (20.6 mg, 0.1 mmol, 1 eq), methyl (triphenyl phosphoranylidene) acetate (50.1 mg, 0.15 mmol, 1.5 eq) and toluene (0.5 ml, 0.2 M). The vessel was flushed with Ar gas for 1 minute and then quickly sealed. The reaction mixture was heated to 120° C. for 14 hours. The reaction mixture was cooled to room temperature, and the mixture was directly purified by FCC (16:1:1 hexanes:EtOAc:CH₂Cl₂) without aqueous work up to yield 116 as a solid (17.6 mg, 67%). Data for 116: Rr 0.375 (5:1 hexanes:EtOAc); mp: 77-80° C.; IR: (thin film) 2953, 1745, 1714, 1599, 1435, 1266, 1198 cm⁻¹; ¹H NMR (700 MHz, CDCl₃) δ 7.86 (d, J=7.7 Hz, 1H), 7.22 (s, 1H), 7.11 (d, J=7.7 Hz, 1H), 3.95 (s, 3H), 3.89 (s, 2H), 3.77 (s, 3H), 2.55 (s, 3H); ¹³C NMR (176 MHz, CDCl₃) δ 169.2, 167.1, 154.2, 152.2, 128.5, 126.8, 125.7, 124.6, 120.0, 106.8, 52.6, 51.9, 34.6, 15.5; HRMS (ESI) m/z: [M+Na]⁺ Calcd for C₁₄H₁₄O₅Na 285.0739; Found 285.0750.

Methyl 7-methyl-2-(((trifluoromethyl)sulfonyl)oxy)benzofuran-4-carboxylate (117): To a solution of methyl 7-methyl-2-oxo-2,3-dihydrobenzofuran-4-carboxylate (108 mg, 0.52 mmol, 1 equiv) in THF (14 ml, 0.038 M) was added LiHMDS (1 M in THF, 1.05 ml, 1.05 mmol, 2 equiv) at −78° C. The mixture was stirred at −78° C. for 1 hour, upon which time trifluoromethanesulfonic anhydride (266 mg, 0.94 mmol, 1.8 equiv) was added dropwise. The mixture was stirred at −78° C. for an additional 1 hour. The mixture was quenched with water (20 ml), extracted with EtOAc (4×30 ml). The combined organic layers were dried over Na₂SO₄, filtered and concentrated. The residue was purified by FCC (40:1 hexanes:EtOAc) to yield 117 as a solid (119 mg, 67%). Data for 117: Rr 0.57 (5:1 hexanes:EtOAc); mp: 39-40° C.; IR: (thin film) 2956, 1722, 1600, 1439, 1230 cm⁻¹; ¹H NMR (700 MHz, CDCl₃) δ 7.94 (d, J=7.7 Hz, 1H), 7.23 (d, J=7.7 Hz, 1H), 7.07 (s, 1H), 3.97 (s, 3H), 2.55 (s, 3H); ¹³C NMR (176 MHz, CDCl₃) δ 166.3, 150.5, 149.5, 127.4, 127.0, 126.8, 126.1, 121.4, 121.1, 119.6, 117.7, 115.9, 94.7, 52.1, 15.2; HRMS (ESI) m/z: [M+H]⁺ Calcd for C₁₂H₁₀O₆F₃S 339.0150; Found 339.0154.

Methyl 7-methylbenzofuran-4-carboxylate (118): A solution of methyl 7-methyl-2-(((trifluoromethyl)sulfonyl)oxy)benzofuran-4-carboxylate (25 mg, 0.075 mmol, 1 equiv) in THF (1.5 ml, 0.05 M) was sparged with Ar gas for 5 minutes. PdCl₂(dppf) (2.8 mg, 0.00375 mmol, 5 mol %), TMEDA (30 mg, 0.255 mmol, 3.4 equiv) and NaBH₄ (9.7 mg, 0.255 mmol, 3.4 equiv) were sequentially added. The mixture was stirred at room temperature for 1.5 hours. The reaction was quenched with brine (15 ml) and extracted with EtOAc (4×15 ml). The combined organic layers were dried over Na₂SO₄, filtered and concentrated. The residue was purified by FCC (40:1 hexanes:EtOAc) to yield 118 as an oil (11.9 mg, 84%). Data for 118: Rr 0.55 (10:1 hexanes:EtOAc); IR: (thin film) 2951, 1715, 1280 cm⁻¹; ¹H NMR (700 MHz, CDCl₃) δ 7.89 (d, J=7.7 Hz, 1H), 7.74 (s, 1H), 7.36 (s, 1H), 7.15 (d, J=7.7 Hz, 1H), 3.97 (s, 3H), 2.59 (s, 3H); ¹³C NMR (176 MHz, CDCl₃) δ 167.1, 154.2, 146.3, 127.4, 127.2, 125.7, 124.7, 120.4, 108.0, 51.9, 15.5; HRMS (ESI) m/z: [M+H]⁺ Calcd for C₁₁H₁₁O₃ 191.0708; Found 191.0714.

Methyl 7-methyl-2-(phenylethynyl)benzofuran-4-carboxylate (119): To a round bottom flask was added a solution of methyl 7-methyl-2-(((trifluoromethyl)sulfonyl)oxy)benzofuran-4-carboxylate (25 mg, 0.075 mmol, 1 equiv) in benzene (0.5 ml, 0.15 M). The headspace of the flask was evacuated and back-filled with Ar three times. Phenylacetylene (15.3 mg, 0.15 mmol, 2 equiv), CuI (2.1 mg, 0.01125 mmol, 15 mol %), diisoproylamine (30 mg, 0.3 mmol, 4 equiv) and Pd(PPh₃)₄ (8.7 mg, 0.0075 mmol, 10 mol %) were added under Ar. The mixture was stirred at room temperature for 1 hour. The reaction was quenched with saturated aqueous NH₄Cl solution (15 ml) and extracted with EtOAc (4×15 ml). The combined organic layers were dried over Na₂SO₄, filtered and concentrated. The residue was purified by FCC (40:1 hexanes:EtOAc) to yield 119 as a solid (19.7 mg, 91%). Data for 119: Rr 0.32 (20:1 hexanes:EtOAc); mp: 71-76° C.; IR: (thin film) 2950, 1716, 1270 cm⁻¹; ¹H NMR (700 MHz, CDCl₃) δ 7.91 (d, J=7.7 Hz, 1H), 7.61 (d, J=6.3 Hz, 2H), 7.59 (s, 1H), 7.41-7.40 (m, 3H), 7.19 (d, J=7.7 Hz, 1H), 3.98 (s, 3H), 2.60 (s, 3H); ¹³C NMR (176 MHz, CDCl₃) δ 166.8, 154.1, 140.1, 131.8, 129.4, 128.5, 127.7, 127.0, 126.3, 125.9, 121.7, 120.3, 113.1, 95.7, 79.6, 52.0, 15.5; HRMS (ESI) m/z: [M+H]⁺ Calcd for C₁₉H₁₅O₃ 291.1021; Found 291.1022.

Methyl 7-methyl-2-phenylbenzofuran-4-carboxylate (120): To a solution of methyl 7-methyl-2-(((trifluoromethyl)sulfonyl)oxy)benzofuran-4-carboxylate (17 mg, 0.05 mmol, 1 equiv) in 1,4-dioxane/water (1:1) (0.5 ml, 0.1 M) was added phenylboronic acid (9.2 mg, 0.075 mmol, 1.5 equiv). The mixture was cooled to 0° C., then Pd(PPh₃)₄ (5.8 mg, 0.005 mmol, 10 mol %) and CsCO₃ (49 mg, 0.15 mmol, 3 equiv) were added at once. The mixture was warmed to room temperature and stirred for 3 hours. Solvent was removed under vacuum. The crude reaction mixture was dissolved in EtOAc, filtered through celite, dried over Na₂SO₃, filtered, and concentrated. The residue was purified by FCC (50:1 hexanes:EtOAc) to yield 120 as a solid (6.5 mg, 49%). Data for 120: Rr 0.25 (20:1 hexanes:EtOAc); mp: 58-61° C.; IR: (thin film) 2953, 1714, 1434, 1269, 1200 cm⁻¹; ¹H NMR (700 MHz, CDCl₃) δ 7.95 (d, J=7.7 Hz, 2H), 7.88 (d, J=7.7 Hz, 1H), 7.64 (s, 1H), 7.48 (t, J=7.7 Hz, 2H), 7.39 (t, J=7.7 Hz, 1H), 7.13 (d, J=7.7 Hz, 1H), 4.00 (s, 3H), 2.64 (s, 3H); ¹³C NMR (176 MHz, CDCl₃) δ 167.2, 157.3, 154.0, 130.2, 129.3, 129.0, 128.9, 126.8, 125.8, 125.3, 124.7, 119.9, 102.8, 51.9, 15.5; HRMS (ESI) m/z: [M+H]⁺ Calcd for C₁₇H₁₅O₃ 267.1021; Found 267.1028.

### Example 7

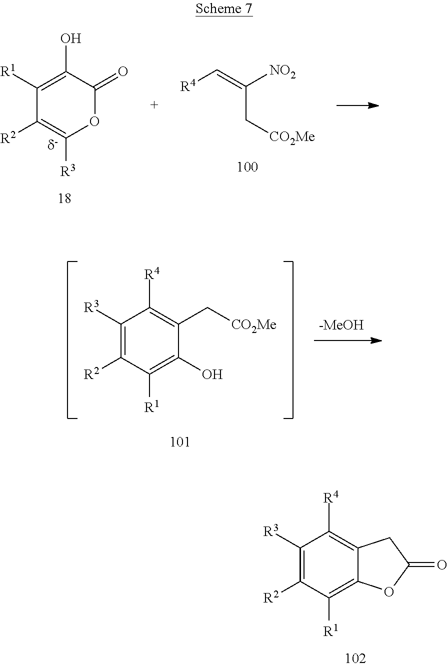

It was speculated that if nitroalkenes bearing a carbomethoxymethyl group (100) were to participate in the reaction, phenols bearing a tethered ester (101) would be formed. Such nitroalkenes are conveniently prepared using standard Henry-type condensations of nitroalkanes.

It was further expected that phenols 101 would cyclize to benzofuranones 102 under the reaction conditions.

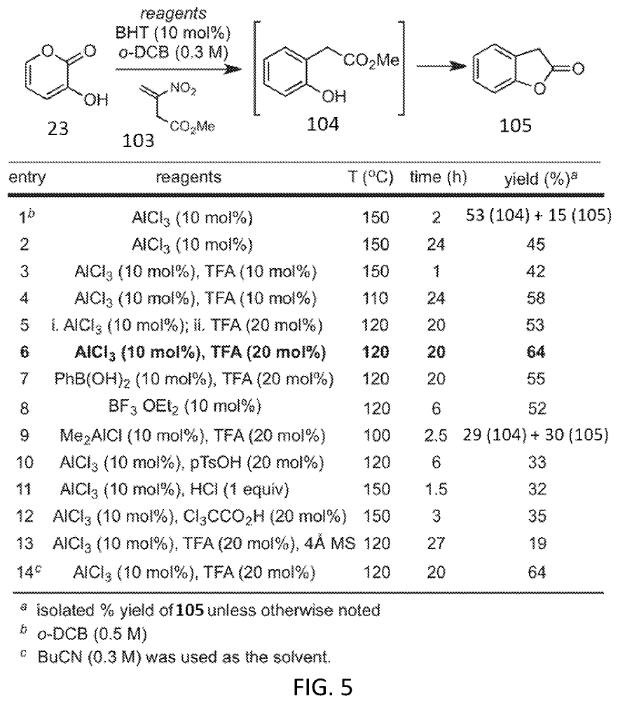

The investigations began with unsubstituted 3-hydroxy-2-pyrone (23) and nitroalkene 103. Conditions disclosed herein for the substituted phenol synthesis (FIG. 5, entry 1) promoted the desired reaction, giving phenol 23 in 53% and the desired benzofuranone product 105 in 15% yield. Allowing the reaction to proceed until there was complete consumption of starting materials 23, 103, and phenol intermediate 104 (TLC check) gave benzofuranone 105 in 45% yield (FIG. 5, entry 2). Apart from the desired product (105), a multitude of minor products were present, but all were produced in less than −5% yield, and were not characterized.

Surprisingly the formation of phenol 104 was faster than its conversion to benzofuranone 105 under the conditions in entries 1 and 2. In a control experiment, it was found that isolation and subjection of phenol 104 to trifluoroacetic acid (TFA) rapidly gave 105 in 70% yield (data not shown). Combining 23 and 103 with both the AlCl₃ Lewis acid and TFA protic acid markedly increased the rate of benzofuranone production (FIG. 5, entry 3). Lowering the reaction temperature gave slightly increased chemical yield of 105 (FIG. 5, entry 4).

Conducting the Diels-Alder-based cascade first with subsequent addition of TFA after consumption of 23 and 103 did not lead to improved yields (FIG. 5, entry 5). Finally, conducting the reaction at 120° C. with 20 mol % of TFA gave an improved yield of benzofuranone 105 (FIG. 5, entry 6).

The benzofuranone synthesis was also evaluated using a variety of different Lewis acids. Boronic acids (FIG. 5, entry 7), boron trifluoride (FIG. 5, entry 8), and other aluminum Lewis acids (FIG. 5, entry 9) all successfully promoted the reaction; however, the overall chemical yield was not improved. The protic acid used to affect ring closure was also varied. Toluenesulfonic acid (FIG. 5, entry 10), hydrochloric acid (FIG. 5, entry 11), and other weak acids such as trichloroacetic acid were evaluated (FIG. 5, entry 12), but none of these acids gave superior results. Addition of molecular sieves in an attempt to sequester the methanol by product gave a substantially decreased yield (FIG. 5, entry 13). Finally, different solvents were evaluated but did not markedly change the chemical yield of the reaction (FIG. 5, entry 14).

The benzofuranone synthesis was then evaluated with other substrate combinations to explore the tolerance of the reaction to substitution. A variety of 4-substituted-3-hydroxy-2-pyrones (18, R¹≠H) were efficiently converted to substituted benzofuranones 102. Methyl (106), branched alkyl (107), and phenyl groups (108) were all well tolerated. Pyrones with multiple substitution also participated in the reaction. 5,6-Disubstituted benzofuran 109 was prepared in modest yield; however, it should be note that this type of benzofuran also is difficult to prepare as a single regioisomer using standard condensation conditions. Trisubstituted benzofuranones 110, 111, and 112 were all prepared in synthetically useful yields. Finally, 4,7-disubstituted benzofurans bearing methyl groups (113), different alkyl groups (114), and electron withdrawing groups (115) were also prepared using the disclosed method.

An advantage of preparing regioselectively substituted benzofuranones is that they are conveniently transformed into the corresponding substituted benzofurans. For example, 115 can be olefinated under standard Wittig conditions to give 2,4,7-trisubstituted benzofuran 116. Benzofuran 115 can also be converted to the corresponding triflate 117. Triflate 117 can be reduced to the parent benzofuran 118. Alternatively, triflate 117 can undergo Sonogashira coupling to give 2-alkynyl benzofuran 119. Suzuki-Miyaura coupling of 117 gives the corresponding 2-aryl benzofuran 120.

In summary, disclosed herein is a novel benzofuranone synthesis from substituted 3-hydroxypyrones and nitroalkenes. The reaction tolerated a variety of substitution patterns, and it resulted in synthetically useful yields. The reaction was completely regiospecific, with the regioisomer of the product being completely predictable through inspection of the starting material substitution. Furthermore, no issues concerning regiochemical mixtures were observed. Finally, the benzofuranone products were conveniently converted into the corresponding substituted benzofurans.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A method, comprising contacting a first compound having a formula I 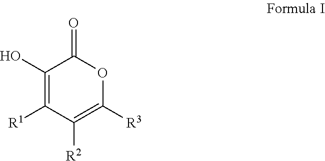
with a second compound according to formula II 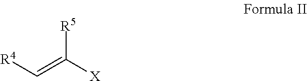
in the presence of a Lewis acid to form a third compound according to formula III 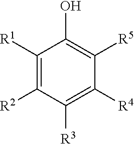
wherein:
each of R¹, R², R³, and R⁴ independently is H, aryl, aliphatic, heterocyclyl, alkoxy, heteroaliphatic, or carboxylic ester;
R⁵ is H, aryl, aliphatic, heterocyclyl, alkoxy, heteroaliphatic, carboxylic ester, or —CH₂CO₂aliphatic; and
X is nitro or SO₂Ph.

2. The method of claim 1, wherein each of R¹, R², R³, R⁴ and R⁵ independently is H, alkyl, cycloalkyl, heteroaryl, heterocycloaliphatic, alkoxy, —CO₂R where R is aliphatic or hydroxyalkyl, or —CH₂CO₂alkyl.

3. The method of claim 1, wherein each of R¹, R², R³, R⁴ and R⁵ independently is H or alkyl.

4. The method of claim 1, wherein:
at least 3 of R¹, R², R³, R⁴ and R⁵ are not H; or
at least 3 of R¹, R², R³, R⁴ and R⁵ are different from each other.

5. The method of claim 1, wherein the Lewis acid is FeCl₃, ZnCl₂, AlCl₃ or a combination thereof.

6. The method of claim 1, wherein the contacting occurs in the presence of a Lewis acid and a radical initiator.

7. The method of claim 6, wherein the radical initiator is butylated hydroxytoluene (BHT), hydroquinone, 2,6-di-tertbutyl phenol, or a combination thereof.

8. The method of claim 1, comprising contacting the compound according to Formula I with the compound according to formula II in the presence of AlCl₃ and butylated hydroxytoluene to form the phenol compound according to formula III.

9. The method of claim 1, wherein R⁵ is —CH₂CO₂aliphtic, and the method further comprises forming a fourth compound according to formula IV

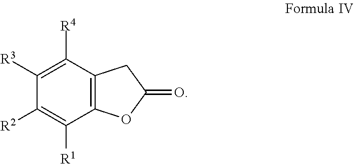

10. The method of claim 9, wherein forming the fourth compound comprises exposing the third compound to a protic acid.

11. The method of claim 10, wherein:
the exposing the third compound to a protic acid comprises forming the third compound in the presence of the protic acid;
the protic acid is haloacetic acid, an aryl sulfonic acid, a hydrohalide acid, or a combination thereof; or
a combination thereof.

12. The method of claim 11, wherein the protic acid is trifluoroacetic acid.

13. The method of claim 9, wherein each of R¹, R², R³, and R⁴ independently is H, alkyl, cycloalkyl, heteroaryl, heterocycloaliphatic, alkoxy, or CO₂R where R is aliphatic or hydroxyalkyl.

14. The method of claim 9, wherein:
each of R¹, R², R³, and R⁴ independently is H or alkyl;
at least 2 of R¹, R², R³, and R⁴ are not H; or
at least 2 of R¹, R², R³, and R⁴ are different from each other.

15. A compound according to formula III made by the method of claim 1 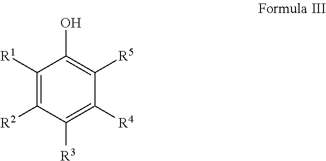
or a salt, or solvate thereof, wherein:
each of R¹, R², R³, R⁴ and R⁵ independently are H, aryl, aliphatic, heterocyclyl, alkoxy, heteroaliphatic, carboxylic ester, or CH₂CO₂aliphatic;
at least 3 of R¹, R², R³, R⁴ and R⁵ are not H and at least two of the non-H substituents are different from each other; and
the compound is not 2,6-dimethyl-3-phenylphenol.

16. The compound according to claim 15, wherein each of R¹, R², R³, R⁴ and R⁵independently is H, alkyl, cycloalkyl, heteroaryl, heterocycloaliphatic, alkoxy, CO₂R where R is aliphatic or hydroxyalkyl, or CH₂CO₂alkyl.

17. The compound according to claim 15, wherein each of R¹, R², R³, R⁴ and R⁵ independently is H or alkyl.

18. The compound according to claim 15, wherein:
at least 4 of R¹, R², R³, R⁴ and R⁵ are not H and at least two of the non-H substituents are different from each other;
each of R¹, R², R³, R⁴ and R⁵ are not H and at least two of the substituents are different from each other; or
each of R¹, R², R³, R⁴ and R⁵ are different from each other.

19. A compound according to formula IV 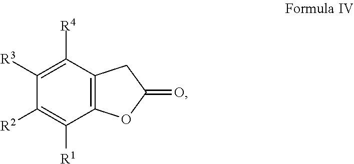
or a salt or solvate thereof, wherein:
each of R¹, R², R³, and R⁴ independently are H, aryl, aliphatic, heterocyclyl, alkoxy, heteroaliphatic, CO₂H or carboxylic ester;
with the proviso that at least two of R¹, R², R³, and R⁴ are not H and one of conditions a), b) and c) applies
a) If exactly two of R¹, R², R³, and R⁴ are not H then they are different from each other;
b) If exactly three of R¹, R², R³, and R⁴ are not H then at least two of the non-H substituents are different from each other; or
c) If all four of R¹, R², R³, and R⁴ are not H then at least two of R¹, R², R³, and R⁴ are different from each other; and
the compound is not
7-acetyl-6-methyl-2-oxo-2,3-dihydrobenzofuran-4,5-dicarboxylic acid;
2,3-Dihydro-6-methyl-2-oxo-4,7-benzofurandicarboxylic acid;
7-Benzoyl-4,6-diphenyl-2(3H)-benzofuranone;
7-Benzoyl-6-(4-methoxyphenyl)-4-phenyl-2(3H)-benzofuranone;
2,3-Dihydro-6-methyl-2-oxo-7-benzofurancarboxaldehyde;
6-methyl-2-oxo-2,3-dihydrobenzofuran-7-carbonitrile;
7-(aminomethyl)-6-methylbenzofuran-2(3H)-one;
7-(2-aminoethyl)-6-methylbenzofuran-2(3H)-one;
2-(6-methyl-2-oxo-2,3-dihydrobenzofuran-7-yl)acetonitrile;
2-(6-methyl-2-oxo-2,3-dihydrobenzofuran-7-yl)acetic acid;
6-methyl-2-oxo-2,3-dihydrobenzofuran-7-carboxylic acid;
2,3-Dihydro-6-methyl-2-oxo-7-benzofuranpropanoic acid;
methyl 2-(6-methyl-2-oxo-2,3-dihydrobenzofuran-7-yl)acetate;
methyl 6-methyl-2-oxo-2,3-dihydrobenzofuran-7-carboxylate;
ethyl 2-(6-methyl-2-oxo-2,3-dihydrobenzofuran-7-yl)acetate;
ethyl 6-methyl-2-oxo-2,3-dihydrobenzofuran-7-carboxylate;
1,1-Dimethylethyl 2,3-dihydro-6-methyl-2-oxo-7-benzofuranpropanoate;
7-(4-Diazo-3-oxobutyl)-6-methyl-2(3H)-benzofuranone;
5-(1,1-Dimethylethyl)-7-methyl-2(3H)-benzofuranone;
7-(1,1-Dimethylethyl)-5-methyl-2(3H)-benzofuranone;
7-(1,1-Dimethylethyl)-5-ethyl-2(3H)-benzofuranone;
7-(1,1-Dimethylethyl)-2,3-dihydro-2-oxo-5-benzofuranacetic acid;
Methyl 2,3-dihydro-5-(1-methylethyl)-2-oxo-7-benzofuranacetate;
7-(1,1-Dimethylethyl)-2,3-dihydro-2-oxo-5-benzofuranpropanoic acid;
Methyl 5-(1,1-dimethylethyl)-2,3-dihydro-2-oxo-7-benzofuranacetate;
methyl 3-(7-(tert-butyl)-2-oxo-2,3-dihydrobenzofuran-5-yl)propanoate;
Octadecyl 7-(1,1-dimethylethyl)-2,3-dihydro-2-oxo-5-benzofuranacetate;
5-Methyl-6-phenyl-2(3H)-benzofuranone;
6-Cyclopentyl-5-methyl-2(3H)-benzofuranone;
6-Cyclohexyl-5-methyl-2(3H)-benzofuranone;
2,3-Dihydro-6-methyl-2-oxo-5-benzofuranpropanoic acid;
Methyl 2,3-dihydro-6-methyl-2-oxo-5-benzofuranpropanoate;
7-Methyl-4-(2-naphthalenyl)-2(3H)-benzofuranone;
2,3-Dihydro-β,4-dimethyl-2-oxo-7-benzofuranpropanoic acid;
4-Methyl-6-(1-methylethyl)-2(3H)-benzofuranone;
Methyl 2,3-dihydro-6-methyl-2-oxo-4-benzofurancarboxylate;
6-methyl-2-oxo-2,3-dihydrobenzofuran-4-carboxylic acid;
2,3-Dihydro-4-methyl-2-oxo-5-benzofuranpropanoic acid;
Methyl 2,3-dihydro-4-methyl-2-oxo-5-benzofuranpropanoate;
Ethyl 2,3-dihydro-4-methyl-2-oxo-5-benzofurancarboxylate; or
5-(4-Diazo-3-oxopentyl)-4-methyl-2(3H)-benzofuranone.

20. A compound selected from: 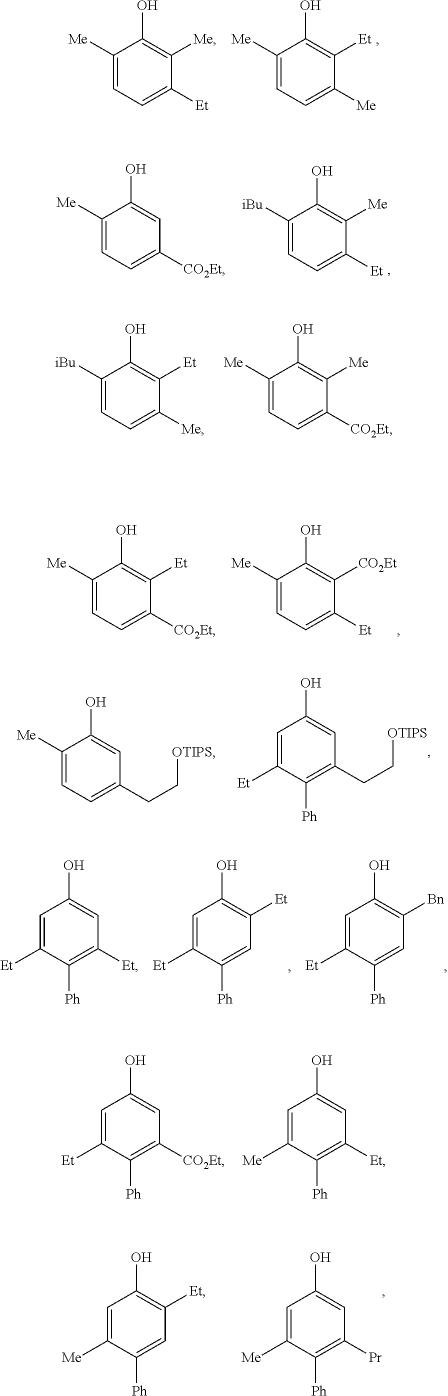 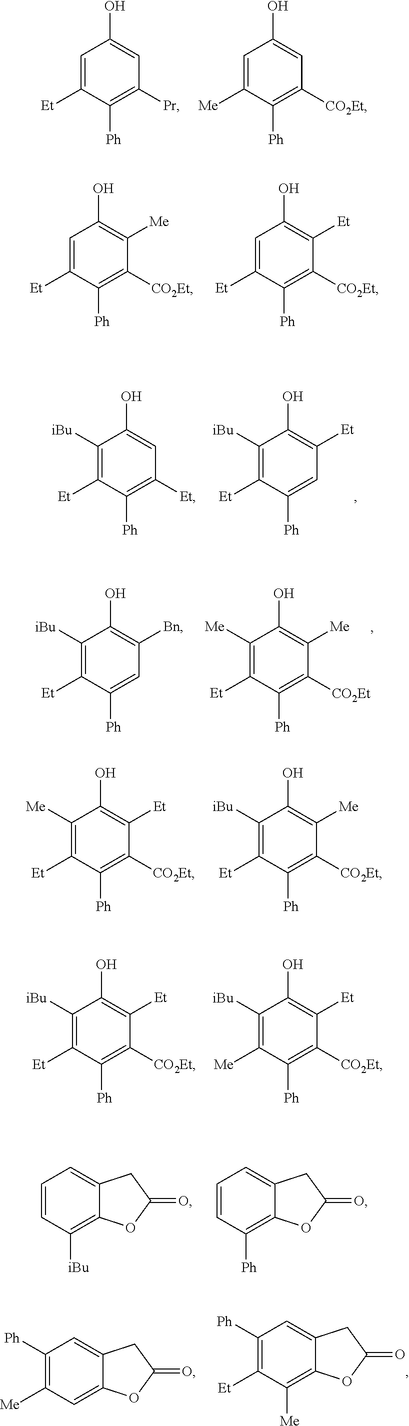 [chemical structure]
